(19) United States
(12) Patent Application Publication
(10) Pub. No.: US 2026/0256770 A1
(43) Pub. Date: Sep. 3, 2026

(54) COMPOSITIONS FOR AND METHODS OF TREATING HEREDITARY AORTOPATHIES

(71) Applicant: Duke University, Durham, NC (US)

(72) Inventors: Joseph Turek, Durham, NC (US); Joseph Nellis, Durham, NC (US)

(21) Appl. No.: 18/717,540

(22) PCT Filed: Dec. 8, 2022
(86) PCT No.: PCT/US2022/081158

§ 371 Date: Jun. 7, 2024

Related U.S. Application Data
(60) Provisional application No. 63/287,595, filed on Dec. 9, 2021.

(51) Int. Cl. A61K 31/4709; A61K 31/4178; A61K 31/4184; A61K 31/4245; A61K 31/522; A61P 9/00; C12Q 1/6883

(52) U.S. Cl. CPC A61K 31/4709; A61K 31/4178; A61K 31/4184; A61K 31/4245; A61K 31/522; A61P 9/00; C12Q 1/6883; C12Q 2600/118; C12Q 2600/158; C12Q 2600/178

(57) ABSTRACT

Disclosed herein are compositions for and methods of treating and/or preventing hereditary aortopathies, treating and/or preventing aortic aneurysms, reducing the need for surgical intervention, slowing and/or inhibiting disease progression, and identifying the risk of aortic aneurysm and/or aortic dissection.

### I. CROSS-REFERENCE TO RELATED APPLICATION

This application claims the benefit of priority to U.S. Provisional Patent Application No. 63/287,595 filed 9 Dec. 2021, which is incorporated by reference herein in its entirety.

### II. REFERENCE TO THE SEQUENCE LISTING

The Sequence Listing submitted 8 Dec. 2022 as an XML file named “22-2058-WO-109726-743261”, created on 8 Dec. 2022 and having a size of 878 kilobytes is hereby incorporated by reference pursuant to 37 C.F.R. § 1.52 (e) (5).

### III. BACKGROUND

Marfan Syndrome (MFS) is a genetic condition that affects males and females in equal numbers and occurs worldwide with no ethnic predisposition. The prevalence has been estimated to be 1 in 5-10,000 individuals in the general population. In the United States, MFS affects approximately 50,000-70,000 people. Because of the difficulty in diagnosing mild cases of MFS, the disorder is probably underdiagnosed, making it difficult to determine its true frequency in the general population.

MFS affects connective tissue, which is the material between cells of the body that gives the tissues form and strength. Connective tissue is found all over the body and multiple organ systems may be affected in individuals with Marfan syndrome. The heart and blood vessels (cardiovascular), skeletal, and eye (ocular) systems are most often affected. Major symptoms include overgrowth of the long bones of the arms and legs, abnormal side-to-side curvature of the spine (scoliosis), indentation or protrusion of the chest wall (pectus deformity), dislocation of the lenses of the eyes (ectopia lentis), nearsightedness (myopia), widening (aneurysm) and tear (dissection) of the main artery that carries blood away from the heart (aorta), floppiness of the mitral valve (mitral valve prolapse) and backward flow of blood through the aortic and mitral valves (aortic and mitral regurgitation). The specific symptoms and the severity of Marfan syndrome vary greatly from person to person. Marfan syndrome is inherited as an autosomal dominant trait, meaning that only one abnormal copy of the Marfan gene inherited from one parent is sufficient to have the condition. Defects or deletions (pathogenic variants) of the fibrillin-1 (FBN1) gene have been shown to cause Marfan syndrome.

For those individuals having MFS, ascending aortic aneurysms (or the dilation of the major artery leaving the heart) is the leading cause of injury and death (e.g., particularly if it tears unexpectedly). Existing medical therapies slow aneurysm progression, but do not eliminate the need for surgery. Moreover, to date, experimental results have failed to translate clinically.

Thus, there is an urgent need for compositions for and methods of treating subjects having or at risk of developing hereditary aortopathy (including, for example, aortic aneurysm and/or aortic dissection.

### IV. BRIEF DESCRIPTION OF THE FIGURES

FIG. 1A-FIG. 1B show TRPC4 expression within the ascending aorta of experimental and control mice. FIG. 1A shows the differential RNA expression with regards TRPC1, TRPC3, TRPC4, and TRPC6 across wild-type (WT), Marfan mice (MFS), and accelerated Marfan mice (MFS+AngII). FIG. 1B shows the relative TRPC4 protein expression between WT, MFS, WT+AngII, and MFS+AngII via Western blot and quantified using Image J.

FIG. 2 shows the differential ascending aortic aneurysm growth in an accelerated Marfan mouse model following administration of pharmacological therapies. Wild-type (WT), baseline non-accelerated Marfan mice (MFS), accelerated MFS mice (aMFS), aMFS mice treated with Losartan (aMFS w/Losartan), aMFS mice treated with ML204, a TRPC4 antagonist (aMFS w/ML204), and aMFS mice treated with both Losartan and ML204 (aMFS w/Losartan and ML204). Mice were treated for two weeks and sacrificed.

FIG. 3 shows the differential ascending aortic aneurysm growth in an accelerated Marfan/TRPC4 knockout mouse model. Mice were treated for two weeks and sacrificed. Measurements were made using transthoracic echocardiography and reported as a percent change in widest diameter from the beginning of the study to the end. Accelerated Marfan mice served as the control (aMFS) and were compared to aMFS receiving Losartan, aMFS mice which were also TRPC4 knockout mice (aMFS:TRPC4^{-/-}), and aMFS:TRPC4^{-/-} mice that were also given Losartan.

FIG. 4 shows TRPC4 expression in human ascending aorta aneurysm sacs following surgical excision and repair. TRPC4 expression in tissue from patients with Marfan and Bicuspid Aortic Valve Syndrome was compared to healthy donor heart aorta. GAPDH is a reference housekeeping protein. There was a 15-fold increase in MFS subjects and a 3-fold increase in biscuspids.

FIG. 5A-FIG. 5C shows the aortic phenotype of C57BL/6J (BL6) and 129S6/SvEvTac (129) MFS mice. FIG. 5A shows aortic root size (mm) in BL6 (n=16) and 129 (n=12) wild-type (WT) mice, and BL6 (n=17) and 129 (n=13) FBN1C1039G/+ (MFS) mice at 2 months of age. FIG. 5B shows aortic root size (mm) in BL6 (n=16) and 129 (n=12) wild-type (WT) mice, and BL6 (n=17) and 129 (n=13) FBN1C1039G/+ (MFS) mice at 6 months of age. FIG. 5C shows the Kaplan-Meier Survival curve to 10 months for BL6 (n=16) and 129 (n=13) wild-type (WT) mice, and BL6 (n=17) and 129 (n=16) FBN1C1039G/+ (MFS) mice. F=female, M=male. Error bars show mean±SEM. *<0.05, **<0.01, ***<0.001, ****<0.0001, NS: non-significant.

FIG. 6 shows the study design for DN and HI MFS mice for Example 1. At each time point, the aortic root of each mouse is imaged. The mice are then randomized into the defined treatment arms. Biochemical control mice are immediately euthanized and used for baseline histology and Western blot analysis of TRPC4, key MMPs, and the TGF-β pathway. All treated animals are imaged at 6 and 12 months.

FIG. 7A-FIG. 7C show the TRPC4 structure. FIG. 7A is a ribbon diagram of mouse including multiple subunits of TRPC4. FIG. 7B shows the ribbon diagram of a single subunit, and FIG. 7C shows a linear diagram depicting structural domains of a TRPC4 monomer.

FIG. 8 shows a schematic of the OSPREY Analytical Package. The package has inputs that include a 3D structure of the target protein, sequence, allowed flexibility, and pairwise energy function. Protein flexibility is estimated using Rigid Dead End Elimination (DEE), improved minimization DEE (iMinDEE), Dead End Elimination with Perturbations (DEEPer), and Coordinate of Atoms by Taylor Series (CATS) model. Energy as Polynomials in Internal Coordinates (EPIC) and Local Unpruned Tuple Expansion (LUTE) algorithms expand energy function capability by allowing for non-pairwise energy functions, basic quantum mechanics and protein binding solvation, all within a continuous DEE framework. Constrained Optimization of Multistate Energies by Tree Search (COMETS) allows for the comparison of various bound and unbound structural states, which are simultaneously processed to yield a list of results based on lowest global conformation energy A* or binding affinity.

FIG. 9 is a schematic of relevant signaling pathways and cascades related to MFS.

### V. BRIEF SUMMARY

Disclosed herein is a method of treating and/or preventing hereditary aortopathy, the method comprising administering to a subject in need thereof a therapeutically effective amount of an angiotensin II receptor antagonist; and administering to the subject a therapeutically effective amount of a TRPC4 antagonist, wherein an existing hereditary aortopathy does not progress.

Disclosed herein is a method of preventing hereditary aortopathy, the method comprising administering to a subject at risk of developing a hereditary aortopathy a therapeutically effective amount of an angiotensin II receptor antagonist; and administering to the subject a therapeutically effective amount of a TRPC4 antagonist, wherein the subject does not develop an aortopathy.

Disclosed herein is a method of treating hereditary aortopathy, the method comprising administering to a subject in need thereof a composition comprising a therapeutically effective amount of an angiotensin II receptor antagonist and a therapeutically effective amount of a TRPC4 antagonist, wherein an existing hereditary aortopathy does not progress.

Disclosed herein is a method of preventing hereditary aortopathy, the method comprising administering to a subject at risk of developing a hereditary aortopathy a composition comprising a therapeutically effective amount of an angiotensin II receptor antagonist and a therapeutically effective amount of a TRPC4 antagonist, wherein the subject does not develop an aortopathy.

Disclosed herein is a method of treating an aortic aneurysm, the method comprising administering to a subject in need thereof a therapeutically effective amount of an angiotensin II receptor antagonist; and administering to the subject a therapeutically effective amount of a TRPC4 antagonist, wherein an existing an aortic aneurysm does not progress.

Disclosed herein is a method of treating an aortic aneurysm, the method comprising administering to a subject at risk of developing a hereditary aortopathy a therapeutically effective amount of an angiotensin II receptor antagonist; and administering to the subject a therapeutically effective amount of a TRPC4 antagonist, wherein the subject does not develop an aortic aneurysm.

Disclosed herein is a method of treating an aortic aneurysm, the method comprising administering to a subject in need thereof a composition comprising a therapeutically effective amount of an angiotensin II receptor antagonist and a therapeutically effective amount of a TRPC4 antagonist, wherein an existing an aortic aneurysm does not progress.

Disclosed herein is a method of treating an aortic aneurysm, the method comprising administering to a subject at risk of developing a hereditary aortopathy a composition comprising a therapeutically effective amount of an angiotensin II receptor antagonist and a therapeutically effective amount of a TRPC4 antagonist, wherein the subject does not develop an aortic aneurysm.

Disclosed herein is a method of reducing the need for surgical intervention, the method comprising administering to a subject in need thereof a therapeutically effective amount of an angiotensin II receptor antagonist; and administering to the subject a therapeutically effective amount of a TRPC4 antagonist, wherein an existing hereditary aortopathy does not progress or wherein the subject does not develop an aortic aneurysm, and wherein surgical intervention is not needed.

Disclosed herein is a method of reducing the need for surgical intervention, the method comprising administering to a subject in need thereof a composition comprising a therapeutically effective amount of an angiotensin II receptor antagonist and a therapeutically effective amount of a TRPC4 antagonist, wherein an existing hereditary aortopathy does not progress or wherein the subject does not develop an aortic aneurysm, and wherein surgical intervention is not needed.

Disclosed herein is a method of slowing and/or inhibiting disease progression, the method comprising administering to a subject having a hereditary aortopathy a therapeutically effective amount of an angiotensin II receptor antagonist; and administering to the subject a therapeutically effective amount of a TRPC4 antagonist, wherein the subject has an aortic aneurysm that does not grow and/or wherein the subject does not develop an aortic dissection.

Disclosed herein is a method of slowing and/or inhibiting disease progression, the method comprising administering to a subject in need thereof a therapeutically effective amount of an angiotensin II receptor antagonist; and administering to the subject a therapeutically effective amount of a TRPC4 antagonist, wherein the subject has an aortic aneurysm that does not grow and/or wherein the subject does not develop an aortic dissection.

Disclosed herein is a method of slowing and/or inhibiting disease progression, the method comprising administering to a subject having a hereditary aortopathy a composition comprising a therapeutically effective amount of an angiotensin II receptor antagonist and a therapeutically effective amount of a TRPC4 antagonist, wherein the subject has an aortic aneurysm that does not grow and/or wherein the subject does not develop an aortic dissection.

Disclosed herein is a method of slowing and/or inhibiting disease progression, the method comprising administering to a subject in need thereof a composition comprising a therapeutically effective amount of an angiotensin II receptor antagonist and a therapeutically effective amount of a TRPC4 antagonist, wherein the subject has an aortic aneurysm that does not grow and/or wherein the subject does not develop an aortic dissection.

Disclosed herein is a method of identifying a subject at risk of aortic aneurysm and/or aortic dissection, the method comprising measuring the level of one or more biomarkers in a biosample obtained from a subject; and if the level of the one or more biomarkers is modulated when compared to a control level, then administering to a subject having a hereditary aortopathy a therapeutically effective amount of an angiotensin II receptor antagonist; and administering to the subject a therapeutically effective amount of a TRPC4 antagonist, wherein risk of the subject developing an aortic aneurysm and/or experiencing an aortic dissection is minimized.

Disclosed herein is a method of identifying a subject at risk of aortic aneurysm and/or aortic dissection, the method comprising measuring the level of one or more biomarkers in a biosample obtained from a subject; and if the level of the one or more biomarkers is modulated when compared to a control level, then administering to a subject having a hereditary aortopathy a composition comprising a therapeutically effective amount of an angiotensin II receptor antagonist and a therapeutically effective amount of a TRPC4 antagonist, wherein risk of the subject developing an aortic aneurysm and/or experiencing an aortic dissection is minimized.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term means “increased risk” is used to mean that a subject has an increased chance of developing an aortic aneurysm and/or aortic dissection when compared a subject known not to have an inherited aortopathy (e.g., a control subject—a subject not having diagnosed with MFS or a subject not having an inherited aortopathy—or a group of control subjects (i.e., a pooled control)). The increased risk may be relative or absolute and may be expressed qualitatively or quantitatively. For example, an increased risk can be expressed as simply determining a subject's proteomic and/or miRNA profile and placing the subject in an “increased risk” category, based upon previous population studies. Alternatively, a numerical expression of the subject's increased risk can be determined based upon the proteomic and/or miRNA profile. As used herein, examples of expressions of an increased risk of developing an aortic aneurysm and/or aortic dissection can include but are not limited to, odds, probability, odds ratio, p-values, attributable risk, relative frequency, positive predictive value, negative predictive value, and relative risk.

For example, the correlation between a subject's proteomic and/or miRNA profile and the likelihood of developing an aortic aneurysm and/or aortic dissection can be measured by an odds ratio (OR) and by the relative risk (RR). If P(R⁺) is the probability of developing an aortic aneurysm and/or aortic dissection for subjects with the risk profile (R) and P(R⁻) is the probability of developing memory impairment for individuals without the risk profile, then the relative risk is the ratio of the two probabilities: RR=P(R⁺)/P(R⁻).

In case-control studies, however, direct measures of the relative risk often cannot be obtained because of sampling design. The odds ratio allows for an approximation of the relative risk for low-incidence diseases and can be calculated: OR=(F⁺/(1−F⁺))/(F/(1−F⁻)), where F⁺ is the frequency of a risk profile in cases studies and F⁻ is the frequency of risk profile in controls. F⁺ and F⁻ can be calculated using the proteomic and/or miRNA profile frequencies of the study.

The attributable risk (AR) can also be used to express an increased risk. The AR describes the proportion of individuals in a population exhibiting memory impairment due to a specific member of the proteomic and/or miRNA risk profile. AR may also be important in quantifying the role of individual components (specific member) in disease etiology and in terms of the public health impact of the individual marker. The public health relevance of the AR measurement lies in estimating the proportion of cases of memory impairment in the population that could be prevented if the profile or individual component were absent. AR may be determined as follows: AR=P_{E}(RR−1)/(P_{E}(RR−1)+1), where AR is the risk attributable to a profile or individual component of the profile, and P_{E} is the frequency of exposure to a profile or individual component of the profile within the population at large. RR is the relative risk, which can be approximated with the odds ratio when the profile or individual component of the profile under study has a relatively low incidence in the general population.

In an aspect, the increased risk of a subject can be determined from p-values that are derived from association studies. Specifically, associations with specific profiles can be performed using regression analysis by regressing the proteomic and/or miRNA profile with developing an aortic aneurysm and/or aortic dissection. In addition, the regression may or may not be corrected or adjusted for one or more factors. The factors for which the analyses may be adjusted include, but are not limited to age, sex, weight, ethnicity, geographic location, fasting state, state of pregnancy or post-pregnancy, menstrual cycle, general health of the subject, alcohol or drug consumption, caffeine or nicotine intake and circadian rhythms, and the subject's FBN1 gene and whether there are one or more genomic aberrations in the FBN1 gene.

Increased risk can also be determined from p-values that are derived using logistic regression. Binomial (or binary) logistic regression is a form of regression which is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring. These analyses are conducted with the program SAS. SAS (“statistical analysis software”) is a general purpose package (similar to Stata and SPSS). Ready-to-use procedures handle a wide range of statistical analyses, including but not limited to, analysis of variance, regression, categorical data analysis, multivariate analysis, survival analysis, psychometric analysis, cluster analysis, and nonparametric analysis.

As used herein, a “Z-score” refers to a standard score that is a very useful statistic because it (a) allows one to calculate the probability of a score occurring within the normal distribution and (b) enables one to compare two scores that are from different normal distributions. The standard score does this by converting (in other words, standardizing) scores in a normal distribution to Z-scores in what becomes a standard normal distribution. A Z-score is a measure of how many standard deviations below or above the population mean a raw score is. A Z-score can be placed on a normal distribution curve. Z-scores range from −3 standard deviations (which would fall to the far left of the normal distribution curve) up to +3 standard deviations (which would fall to the far right of the normal distribution curve).

As used herein, “Cohen's D” or “standardized mean difference” refers to one of the most common ways to measure effect size. An effect size is how large an effect is. For example, medication A has a larger effect than medication B. While a p-value can tell you if there is an effect, it won't tell you how large that effect is. Cohen's D specifically measures the effect size of the difference between two means. The formula for Cohen's D (for equally sized groups) is: d=(M1−M2)/Spooled, where M1=mean of group 1, M2=mean of group 2, spooled=pooled standard deviations for the two groups. The formula is: √[(s12+s22)/2].

As used herein, the term “subject” can refer a human being or a human patient. The term “subject” also includes domesticated animals (e.g., cats, dogs, etc.), livestock (e.g., cattle, horses, pigs, sheep, goats, etc.), and wild-type and/or recombinant laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient in need of medical treatment and/or surgical intervention due to a hereditary aortopathy. In an aspect, a subject can be a human patient in need of medical treatment and/or surgical intervention due to an aortic aneurysm and/or aortic dissection.

As used herein, the term “diagnosed” means having been subjected to an examination by a person of skill, for example, a physician, and found to have a condition that can be diagnosed or treated with a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof, treated by one or more of the disclosed methods, or the combination thereof. For example, “diagnosed with a hereditary aortopathy” means having been subjected to an examination by a person of skill, for example, a physician, and found to have a condition (such as, for example, a hereditary aortopathy) that can be treated with a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof, by one or more of the disclosed methods, or the combination thereof. Also, for example, “diagnosed with a aortic aneurysm” means having been subjected to an examination by a person of skill, for example, a physician, and found to have a condition (such as, for example, a aortic aneurysm) that can be treated with a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof, by one or more of the disclosed methods, or the combination thereof. In an aspect, an examination can be physical, can involve various tests (e.g., blood tests, genotyping, biopsies, etc.), scans (e.g., CT scans, PET scans, etc.), and assays (e.g., enzymatic assay), or a combination thereof.

A “patient” refers to a subject needing medical treatment and/or surgical intervention due to a hereditary aortopathy. In an aspect, a patient can refer to a subject that has been diagnosed with a hereditary aortopathy (such as, for example, due to MFS) and is seeking treatment. For example, a subject can be identified as having a need for medical treatment and/or surgical intervention due to a hereditary aortopathy based upon an earlier diagnosis by a person of skill and thereafter subjected to treatment for this need. In an aspect, the identification can be performed by a person different from the person making the diagnosis.

As used herein, “inhibit,” “inhibiting”, and “inhibition” mean to diminish or decrease an activity, response, condition, severity, disease, or other biological parameter (such as, for example, aortic diameter). This can include, but is not limited to, the complete ablation of the activity, response, condition, severity, disease, or otheran also include, for example, a 10% inhibition or reduction in the activity, expression level, response, condition, severity, disease, or other biological parameter as compared to the native or control level (e.g., a subject not having a hereditary aortopathy or a subject not having MFS). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not having a hereditary aortopathy or a subject not having MFS). In an aspect, the inhibition or reduction can be 0-25%, 25-50%, 50-75%, or 75100% as compared to native or control levels.

As used herein, “aortic valve regurgitation” or “aortic regurgitation” refers to the condition that occurs when the aortic valve does not close tightly. Here, some of the blood pumped out of left ventricle leaks backward and prevents the heart from efficiently pumping blood to the rest of the body.

As used herein, “aneurysm” can refer to a bulge or “ballooning” in the wall of an artery. Arteries are blood vessels that carry oxygen-rich blood from the heart to other parts of the body. If an aneurysm grows large, it can burst and cause dangerous bleeding or even death. Most aneurysms are in the aorta, the main artery that runs from the heart through the chest and abdomen. There are two types of aortic aneurysm: (i) thoracic aortic aneurysms (TAA)—these occur in the part of the aorta running through the chest; and (ii) abdominal aortic aneurysms (AAA)—these occur in the part of the aorta running through the abdomen.

As used herein, the term “tissue” refers to any part of the body exercising a specific function including organs and cells or parts thereof, for example, cell lines or organelle preparations. Other examples include conduit vessels such as arteries or veins or circulatory organs such as the heart, respiratory organs such as the lungs, urinary organs such as the kidneys or bladder, digestive organs such as the stomach, liver, pancreas or spleen, reproductive organs such as the scrotum, testis, ovaries or uterus, neurological organs such as the brain, germ cells such as spermatozoa or ovum and somatic cells such as skin cells, heart cells (i.e., myocytes), nerve cells, brain cells or kidney cells. In an aspect, “tissue” refers to portions dissected from a donor heart (e.g., valve, septal tissue, root, etc.).

As used herein, the term “organ” refers to any part of the body exercising a specific function including tissues and cells or parts thereof, for example, endothelium, epithelium, blood brain barrier, cell lines or organelle preparations. Other examples include circulatory organs such as the blood vessels, heart, respiratory organs such as the lungs, urinary organs such as the kidneys or bladder, digestive organs such as the stomach, liver, pancreas or spleen, reproductive organs such as the scrotum, testis, ovaries or uterus, neurological organs such as the brain, germ cells such as spermatozoa or ovum and somatic cells such as skin cells, heart cells i.e., myocytes, nerve cells, brain cells or kidney cells.

The words “treat” or “treating” or “treatment” include palliative treatment, that is, treatment designed for the relief of symptoms rather than the curing of the infection, disease, pathological condition, or disorder (such as, for example, a hereditary aortopathy, an aortic aneurysm, an aortic dissection, and/or MFS); preventative treatment, that is, treatment directed to minimizing or partially or completely inhibiting the development of the associated infection, disease, pathological condition, or disorder (such as, for example, a hereditary aortopathy, an aortic aneurysm, an aortic dissection, and/or MFS); and supportive treatment, that is, treatment employed to supplement another specific therapy directed toward the improvement of the associated infection, disease, pathological condition, or disorder (such as, for example, a hereditary aortopathy, an aortic aneurysm, an aortic dissection, and/or MFS). In an aspect, the terms cover any treatment of a subject, including a mammal (e.g., a human), and includes: (i) preventing the undesired infection, physiological change, disease, pathological condition, or disorder (such as, for example, a hereditary aortopathy, an aortic aneurysm, an aortic dissection, and/or MFS) from occurring in a subject that can be predisposed to the disease but has not yet been diagnosed as having it; (ii) inhibiting the infection, physiological change, disease, pathological condition, or disorder (such as, for example, a hereditary aortopathy, an aortic aneurysm, an aortic dissection, and/or MFS), i.e., arresting its development; or (iii) relieving the infection, physiological change, disease, pathological condition, or disorder (such as, for example, a hereditary aortopathy, an aortic aneurysm, an aortic dissection, and/or MFS), i.e., causing regression of the disease. For example, in an aspect, treating can reduce the severity of a dysfunction cardiac valve in a subject by 1%-100% as compared to a control (such as, for example, an individual not having a hereditary aortopathy, an aortic aneurysm, an aortic dissection, and/or MFS). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a hereditary aortopathy, an aortic aneurysm, an aortic dissection, and/or MFS. For example, treating can reduce one or more symptoms of a dysfunctional cardiac valve in a subject by 1%-100% as compared to a control (such as, for example, an individual not having a hereditary aortopathy, an aortic aneurysm, an aortic dissection, and/or MFS). It is understood that treatment does not necessarily refer to a cure or complete ablation or eradication of a hereditary aortopathy, an aortic aneurysm, an aortic dissection, and/or MFS. However, in an aspect, treatment can refer to a cure or complete ablation or eradication of aortopathy, aortic aneurysm, and/or aortic dissection.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a fatal aortic dissection or death is intended. The words “prevent”, “preventing”, and “prevention” also refer to prophylactic or preventative measures for protecting or precluding a subject (e.g., an individual) not having the infection, physiological change, disease, pathological condition, or disorder or related complication (such as, for example, a hereditary aortopathy, an aortic aneurysm, an aortic dissection, and/or MFS) from progressing to that complication (e.g., a fatal aortic dissection and/or death).

As used herein, the terms “administering” and “administration” refer to any method of providing to a subject and/or a patient one or more of a disclosed angiotensin II receptor antagonist and/or a disclosed TRPC4 antagonist, a disclosed composition comprising a disclosed angiotensin II receptor antagonist and/or a disclosed TRPC4 antagonist, a disclosed pharmaceutical formulation, a disclosed therapeutic agent, and/or any combination thereof. Such methods are well known to those skilled in the art and include, but are not limited to, the following: oral administration, transdermal administration, administration by inhalation, nasal administration, topical administration, in utero administration, intratumoral administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can be continuous or intermittent. Administration can comprise a combination of routes.

In an aspect, the skilled person can determine an efficacious dose, an efficacious schedule, and an efficacious route of administration of one or more of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, the skilled person can also alter, change, or modify an aspect of an administering step to improve efficacy of a disclosed angiotensin II receptor antagonist and/or a disclosed TRPC4 antagonist, a disclosed composition comprising a disclosed angiotensin II receptor antagonist and/or a disclosed TRPC4 antagonist.

By “determining the amount” is meant a quantification of a particular analyte (e.g., biomarker for a phenotypic switch) or a particular clinical outcome or parameter.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during the course of a common treatment schedule.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed TRPC4 antagonist, or a disclosed angiotensin II receptor antagonist, or a disclosed pharmaceutical formulation comprising either or both) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., a disclosed TPRC4 antagonist, or a disclosed angiotensin II receptor antagonist, or a disclosed pharmaceutical formulation comprising either or both) to a subject in need thereof.

The term “contacting” as used herein refers to bringing one or more of a disclosed angiotensin II receptor and/or a disclosed TRPC4 antagonist, a disclosed composition comprising a disclosed angiotensin II receptor and/or a disclosed TRPC4 antagonist, disclosed immunosuppressive agents, pharmaceutical formulations, or therapeutic agents, or any combination thereof together with a target area or intended target area (e.g., such as an aortic aneurysm) in such a manner that the disclosed immunosuppressive agents, pharmaceutical formulations, or therapeutic agents, or any combination thereof can exert an effect on the intended target or targeted area either directly or indirectly.

As used herein, “effective amount” and “amount effective” can refer to an amount that is sufficient to achieve the desired result such as, for example, the treatment and/or prevention of an aortopathy. As used herein, the terms “effective amount” and “amount effective” can refer to an amount that is sufficient to achieve the desired an effect on an undesired condition (e.g., reducing, preventing, treating, and/or slowing the progression of an aortic aneurysm, an aortic dissection, and/or death). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed angiotensin II receptor and/or a disclosed TRPC4 antagonist, a disclosed composition comprising a disclosed angiotensin II receptor and/or a disclosed TRPC4 antagonist, a disclosed immunosuppressive agent, pharmaceutical formulation, or therapeutic agent, or any combination thereof that (i) treats the particular infection, disease, condition, or disorder (such as a hereditary aortopathy), (ii) attenuates, ameliorates, or eliminates one or more symptoms of the particular infection, disease, condition, or disorder (such as a hereditary aortopathy), or (iii) delays the onset of one or more symptoms of the particular infection, disease, condition, or disorder (such as a hereditary aortopathy) described herein. The specific therapeutically effective dose level for any particular patient will depend upon a variety of factors including the particular infection, disease, condition, or disorder (such as a hereditary aortopathy) being treated and the severity of the particular infection, disease, condition, or disorder (such as a hereditary aortopathy); the disclosed biotherapeutics, the disclosed pharmaceutical formulations, or any combination thereof employed in the disclosed methods; the age, body weight, general health, sex and diet of the patient; the time of administration; the route of administration; the rate of excretion of the disclosed biotherapeutics, the disclosed pharmaceutical formulations, or any combination thereof employed; the duration of the treatment; other drugs used in combination or coincidental with the disclosed biotherapeutics, the disclosed pharmaceutical formulations, or any combination thereof, and other like factors well known in the medical arts. For example, it is well within the skill of the art to start doses of the disclosed biotherapeutics, the disclosed pharmaceutical formulations, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed biotherapeutics, the disclosed pharmaceutical formulations, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of an aortic aneurysm, aortic dissection, and/or death.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, the term “package insert” is used to refer to instructions customarily included in commercial packages of therapeutic products, that contain information about the indications, usage, dosage, administration, contraindications and/or warnings concerning the use of such therapeutic products.

Disclosed are the components to be used to prepare the disclosed immunosuppressive agents or the disclosed pharmaceutical formulations used within the methods disclosed herein.

These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Marfan Syndrome

### i. BACKGROUND

Marfan syndrome (MFS) is a genetic disorder that affects connective tissue, which is the material between cells of the body that gives the tissues form and strength. Connective tissue is found all over the body and multiple organ systems may be affected in individuals with Marfan syndrome. The heart and blood vessels (cardiovascular), skeletal, and eye (ocular) systems are most often affected. Major symptoms include overgrowth of the long bones of the arms and legs, abnormal side-to-side curvature of the spine (scoliosis), indentation or protrusion of the chest wall (pectus deformity), dislocation of the lenses of the eyes (ectopia lentis), nearsightedness (myopia), widening (aneurysm) and tear (dissection) of the main artery that carries blood away from the heart (aorta), floppiness of the mitral valve (mitral valve prolapse) and backward flow of blood through the aortic and mitral valves (aortic and mitral regurgitation). The specific symptoms and the severity of Marfan syndrome vary greatly from person to person. Marfan syndrome is inherited as an autosomal dominant trait, meaning that only one abnormal copy of the Marfan gene inherited from one parent is sufficient to have the condition. Defects or deletions (pathogenic variants) of the fibrillin-1 (FBN1) gene have been shown to cause Marfan syndrome.

### ii. Signs and Symptoms

The specific symptoms of Marfan syndrome vary greatly from person to person. Some individuals will develop only a few mild or isolated symptoms; others will develop more serious complications. In most cases, Marfan syndrome progresses as individuals grow older. In some infants, Marfan syndrome may cause severe, rapidly progressive complications during infancy, often quickly affecting multiple organ systems early in life. Marfan syndrome can potentially affect many systems of the body including the heart, blood vessels, skeleton, eyes, lungs, and skin.

Individuals with Marfan syndrome often develop distinct physical findings often including an abnormally thin physique and disproportionately long, slender arms and legs (dolichostenomelia) due to overgrowth of the long bones. In addition, affected individuals usually have abnormally long, slender fingers (arachnodactyly). People with Marfan syndrome are usually very tall and thin in comparison to unaffected family members but not necessarily in comparison to the general population. They can lack muscle tone (hypotonia) and have little fat under the skin (subcutaneous fat).

A variety of skeletal malformations affect individuals with Marfan syndrome including overgrowth of the ribs, which can push the breastbone (sternum) inward resulting in a sunken chest (pectus excavatum) or outward resulting in a protruding chest (pectus carinatum). Additional symptoms include abnormally loose or flexible joints (joint hypermobility), flat feet (pes planus), fingers that are permanently bent or “fixed” and cannot extend or straighten fully (camptodactyly or clinodactyly), and reduced extension of the elbow. In some cases, the joints may be unaffected or may become tight and stiff (contractures). Some individuals have an abnormally deep hip socket (acetabulum) with deep insertion of the head of the long bone (femur) of the leg (protrusio acetabulae) and signs of bone erosion. Many individuals with Marfan syndrome develop spinal abnormalities such as progressive curving of the spine (scoliosis) that may be mild or severe. Scoliosis may be associated with back pain in some affected individuals. In children, skeletal abnormalities may progress rapidly during phases of rapid growth, such as adolescence.

Individuals with Marfan syndrome may have several distinct facial features including a long, narrow skull (dolichocephaly), deep-set eyes (enophthalmos), an abnormally small jaw (micrognathia) that may be recessed farther back than normal (retrognathia), abnormally flat cheek bones (malar hypoplasia), and an abnormal downward slant to the eyes (downward slanting palpebral fissures). Affected individuals may also exhibit a highly-arched roof of the mouth (palate), teeth that are crowded together and upper and lower teeth that do not meet (align) properly when biting (malocclusion).

Individuals with Marfan syndrome may have significant cardiovascular problems such as a common heart defect known as mitral valve prolapse. The mitral valve is located between the left upper and left lower chambers (left atrium and left ventricle, respectively) of the heart. Mitral valve prolapse occurs when one or both of the flaps (cusps) of the mitral valve bulge or collapse backward (prolapse) into the left upper chamber (atrium) of the heart during ventricular contraction. In some cases, this may allow leakage or the backward flow of blood from the left lower chamber of the heart (ventricle) back into the left atrium (mitral regurgitation). Often no associated symptoms are apparent (asymptomatic). However, in other cases, mitral valve prolapse can result in chest pain, abnormal heart rhythms (arrhythmias), or evidence of inadequate heart function (congestive heart failure, most often in association with prolonged and severe mitral regurgitation).

Additional cardiovascular findings include widening (aneurysm) and degeneration of the main artery that carries blood away from the heart (aorta), tearing (dissection) of the aorta so that blood seeps between the inner and outer layers of the aortic wall, and backward flow of blood from the aorta into the lower left chamber (ventricle) of the heart (aortic regurgitation). If severe and left untreated, these heart abnormalities associated with Marfan syndrome can cause life-threatening complications such as rupture of the aorta and congestive heart failure. Some individuals may develop widening of the main artery of the lungs (pulmonary artery dilatation). This typically does not cause any problems in people with Marfan syndrome.

Individuals with Marfan syndrome commonly develop abnormalities of the eyes, especially nearsightedness (myopia), which may develop early in childhood and become progressively worse. Approximately 60 percent of individuals develop displacement of the lenses of the eyes away from the center of the eye (ectopia lentis). Ectopia lentis may occur at birth or later in life and may remain stable or become progressively worse.

Additional issues affecting the eyes in Marfan syndrome include an abnormally flat cornea (the front portion of the eyes through which light passes), underdevelopment of the colored portion of the eye (hypoplastic iris), and detachment of the nerve-rich membrane (retina) lining the back of the eyes. Some individuals with Marfan syndrome are at risk for the early development of clouding of the lenses of the eyes (cataracts) or increased pressure and/or associated changes in the eyes (glaucoma). If left untreated, eye abnormalities can result in vision loss.

Some individuals with Marfan syndrome may develop distended air pockets near the top of the lungs (apical pulmonary blebs), which can predispose individuals to a leak of air within the chest cavity and lung collapse that occurs for no readily apparent reason (spontaneous pneumothorax). In some cases, pneumothorax can recur in the same lung or even the opposite lung (recurrent pneumothorax).

Some affected individuals may develop widening or bulging of the sac (dura) that surrounds the spinal cord (dural ectasia). This condition usually does not cause symptoms (asymptomatic), but has been associated with lower back pain and can cause pinching of a nerve leading to abnormal sensations or muscle performance in the legs. Affected individuals may also developed stretch marks (striae atrophicae) of the skin without an obvious cause. Some affected individuals may have an inguinal, umbilical or surgical hernia, in which a weakened portion of the pelvic or abdominal wall shows external bulging and even protrusion of a small segment of the intestines.

Researchers have identified a subset of individuals with symptoms that are extremely similar to those associated with Marfan syndrome; however, these individuals have changes in different genes. Of equal importance, these individuals have now been recognized to be at risk for numerous features that are not seen in Marfan syndrome caused by mutations in FBN1. While a subset of these patients were historically designated as having Marfan syndrome type II, it is now more common practice to specify alternative diagnoses including Loeys-Dietz syndrome or Shprintzen-Goldberg syndrome (see the Related Disorders section below). People with a Marfan-like condition caused by mutations in a gene other than FBN1 may require specialized counseling, imaging protocols and management.

### iii. Causes

Marfan syndrome is caused by defects or deletions (mutations) of the fibrillin-1 (FBN1) gene. Not everyone who has a mutation of this gene develops Marfan syndrome. Some changes do not alter the function of the gene or protein and therefore do not cause a medical problem. Other changes in the FBN1 gene can cause conditions that are distinct from Marfan syndrome.

Fibrillin-1 is a component of structures called microfibrils, which are fiber-like structures that are part of the extracellular matrix, a complex material that surrounds and connects cells throughout the body. Researchers believe fibrillin-1 plays an important role in maintaining the strength and structural integrity of the connective tissue. Without fibrillin-1, connective tissue may be weak. Fibrillin-1 also influences the activity of molecules that instruct cells how to behave (growth factors), including a specific growth factor called transforming growth factor-β (TGF-β). Marfan syndrome is inherited as an autosomal dominant condition. Dominant genetic disorders occur when only a single copy of an abnormal gene is sufficient to cause a particular disease. The abnormal gene can be inherited from either parent or can be the result of a new mutation (gene change) in the affected individual. In approximately 25 percent of Marfan syndrome cases, the disease-causing DNA change occurs as the result of a new mutation. The risk of passing the abnormal gene from affected parent to offspring is 50% for each pregnancy. The risk is the same for males and females. The children of an individual with Marfan syndrome caused by a new mutation have a 50% chance of inheriting this gene change and hence Marfan syndrome from their affected parent.

A disorder that includes many features of Marfan syndrome (MFS) called Loeys-Dietz syndrome (LDS) can be caused by mutations in at least 5 different genes that influence the activity of TGF-β (TGFβR1, TGFβR2, SMAD3, TGFB2, and TGFB3). Another condition called Shprintzen-Goldberg syndrome or SGS includes many features of MFS and most features of LDS, but also problems with learning (intellectual disability). SGS is caused by mutations in another gene that regulates TGF-β activity called SKI.

### iv. FBN1

Fibrillin 1 (FBN1) encodes a member of the fibrillin family of proteins. In humans, there are three different genes (FBN1, FBN2, and FBN3) encoding fibrillins. Fibrillins are large (~350,000 MW) structural macromolecules that contribute to the integrity and function of all connective tissues. They are considered to be “structural macromolecules” because, like the collagens, the fibrillins form fibers that are visible in transmission electron micrographs. The encoded preproprotein is proteolytically processed to generate two proteins including the extracellular matrix component fibrillin-1 and the protein hormone asprosin. Fibrillin-1 is an extracellular matrix glycoprotein that serves as a structural component of calcium-binding microfibrils. These microfibrils provide force-bearing structural support in elastic and nonelastic connective tissue throughout the body. Asprosin, secreted by white adipose tissue, has been shown to regulate glucose homeostasis. Mutations in this gene are associated with Marfan syndrome and the related MASS phenotype, as well as ectopia lentis syndrome, Weill-Marchesani syndrome, Shprintzen-Goldberg syndrome and neonatal progeroid syndrome.

FBN1 is a structural component of the 10-12 nm diameter microfibrils of the extracellular matrix, which conveys both structural and regulatory properties to load-bearing connective tissues. Fibrillin-1-containing microfibrils provide long-term force bearing structural support. In tissues such as the lung, blood vessels and skin, microfibrils form the periphery of the elastic fiber, acting as a scaffold for the deposition of elastin. In addition, microfibrils can occur as elastin-independent networks in tissues such as the ciliary zonule, tendon, cornea and glomerulus where they provide tensile strength and have anchoring roles. Fibrillin-1 also plays a key role in tissue homeostasis through specific interactions with growth factors, such as the bone morphogenetic proteins (BMPs), growth and differentiation factors (GDFs) and latent transforming growth factor-beta-binding proteins (LTBPs), cell-surface integrins and other extracellular matrix protein and proteoglycan components. Regulates osteoblast maturation by controlling TGF-beta bioavailability and calibrating TGF-beta and BMP levels, respectively (By similarity). Negatively regulates osteoclastogenesis by binding and sequestering an osteoclast differentiation and activation factor TNFSF11. This leads to disruption of TNFSF11-induced Ca(²⁺) signaling and impairment of TNFSF11-mediated nuclear translocation and activation of transcription factor NFATC1 which regulates genes important for osteoclast differentiation and function. Mediates cell adhesion via its binding to cell surface receptors integrins ITGAV:ITGB3 and ITGA5:ITGB1. Binds heparin and this interaction has an important role in the assembly of microfibrils.

Asprosin is an adipokine secreted by white adipose tissue that plays an important regulatory role in the glucose metabolism of liver, muscle and pancreas. Hormone that targets the liver in response to fasting to increase plasma glucose levels. Binds the olfactory receptor OR4M1 at the surface of hepatocytes and promotes hepatocyte glucose release by activating the protein kinase A activity in the liver, resulting in rapid glucose release into the circulation. May act as a regulator of adaptive thermogenesis by inhibiting browning and energy consumption, while increasing lipid deposition in white adipose tissue (By similarity). Also acts as an orexigenic hormone that increases appetite: crosses the blood brain barrier and exerts effects on the hypothalamus (By similarity). In the arcuate nucleus of the hypothalamus, asprosin directly activates orexigenic AgRP neurons and indirectly inhibits anorexigenic POMC neurons, resulting in appetite stimulation (By similarity).

Since the identification of FBN1 as the causal gene for Marfan syndrome, 1847 different mutations in 3044 DNA samples have been reported, according to the currently most extensive FBN1 mutation database (http://www.umd.be/FBN1/-last updated Aug. 28, 2014). Mutations are found throughout the entire length of the gene, and only 12% of all reported FBN1 mutations are recurrent. It is likely that the number of known mutations is much larger, since not all FBN1 mutations that have been identified worldwide have been reported or entered into this database. Penetrance of FBN1 mutations is extremely high, but age-dependent, and no example of nonpenetrance has been documented in hundreds of pedigrees. Mutations in the FBN1 gene include many different types. Missense mutations are most frequently encountered (about ⅔ of cases). These commonly substitute cysteine residues that form disulfide bonds within one of the cbEGF or 8-Cys domains, but missense mutations creating novel cysteine residues in these modules are also common. About one quarter of missense mutations affect modules other than cbEGF domains. Small insertions, deletions, or duplications represent around 10-15% of all reported mutations, the majority of which will create a premature termination codon (PTC). Another 10-15% of reported mutations consist of various classes of splicing errors, most commonly affecting canonical splice sequences at exon/intron boundaries. Many FBN1 splice site mutations result in in-frame exon skipping, such that the mutant fibrillin-1 lacks an entire cbEGF domain. Some exon-skipping mutations in FBN1 result in a frameshift and reduced mutant RNA levels caused by nonsense-mediated decay of the mutant transcript. Larger rearrangements, including both deletions and insertions, have been reported in a minority of Marfan patients; entire gene deletions are rare.

### v. MFS Mouse Model

The FBN1C^{1039G/+} heterozygous mice (Jackson Laboratory, Bar Harbor, ME) with a mutation in the FBN1 (fibrillin 1) gene are used as a validated MFS model. The FBN1/C^{1039G/+} mouse has been widely used to investigate the sequelae of MFS. This model can take months for significant aneurysms to form and results in a low frequency of cardiomyopathy.

The Examples provided infra use a novel murine MFS model of accelerated aortic aneurysm formation and cardiomyopathy. This MFS uses two well-characterized in vivo systems for studying aortopathy and cardiomyopathy, namely, the FBN1 mutant MFS mouse and osmotically delivered angiotensin II infusion. In this accelderated model, significant aortic aneurysms were observed as early as 10 days after angiotensin II pump implantation. Furthermore, the acceleration of the aortopathy was so profound that one third of the treatment group died at 14 days and more than half died at 28 days after implant. Overwhelmingly, these deaths were a result of ascending aortic aneurysm rupture or dissection. Histopathologic assessment of affected mice revealed classic elastin fragmentation. Of note, a hypertensive effect of angiotensin II was not a contributing factor to aneurysmal formation, as blood pressures were not elevated in the treatment group, and angiotensin II-infused wild-type mice did not form aneurysms over the treatment period. Similarly, the accelerated MFS model described and used herein produced a dilated cardiomyopathy as early as 14 days after angiotensin II minipump installation, as determined by echocardiographic measurements of end-diastolic volume and ejection fraction, heart mass-to-body weight ratios, and hematoxylin and eosin tissue staining patterns consistent with the diagnosis. After 14 days of pump installation, 60% of surviving angiotensin II-infused MFS mice met a defined echocardiographic criteria for dilated cardiomyopathy. Importantly, nearly half (4 of 9) of these cardiomyopathie mice revealed either no or mild aortic insufficiency indicating our model expresses a reproducible primary dilated cardiomyopathy intrinsic to MFS.

### C. TRPC4

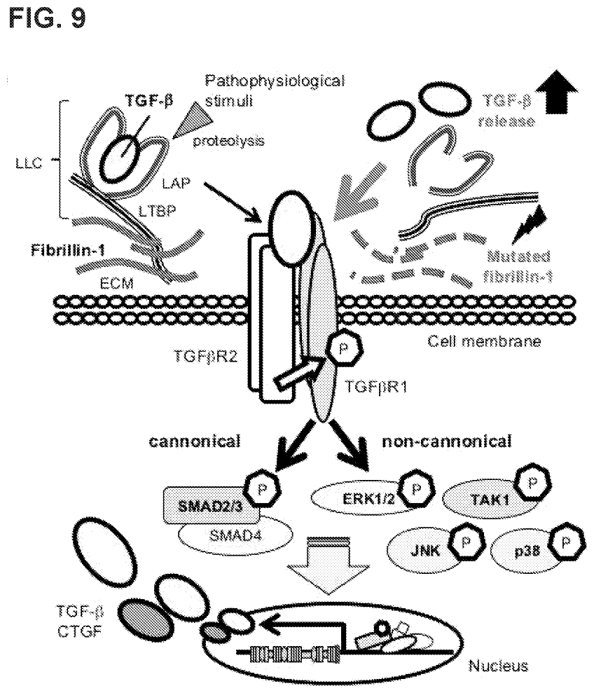

Transient receptor potential cation channel subfamily C member 4 or TRPC4 encodes a member of the canonical subfamily of transient receptor potential cation channels. (FIG. 9) The encoded protein forms a non-selective calcium-permeable cation channel that is activated by Gq-coupled receptors and tyrosine kinases and plays a role in multiple processes including endothelial permeability, vasodilation, neurotransmitter release and cell proliferation. Single nucleotide polymorphisms in this gene may be associated with generalized epilepsy with photosensitivity. Alternatively spliced transcript variants encoding multiple isoforms have been observed for this gene. TRPC4 proteins comprise six transmembrane domains, a putative pore-forming region, and an intracellularly located amino- and carboxy-terminus. Among eleven splice variants identified so far, TRPC4α and TRPC4β are the most abundantly expressed and functionally characterized. TRPC4 is expressed in various organs and cell types including the soma and dendrites of numerous types of neurons; the cardiovascular system including endothelial, smooth muscle, and cardiac cells; myometrial and skeletal muscle cells; kidney; and immune cells such as mast cells. Both recombinant and native TRPC4-containing channels differ tremendously in their permeability and other biophysical properties, pharmacological modulation, and mode of activation depending on the cellular environment. They vary from inwardly rectifying store-operated channels with a high Ca²⁺ selectivity to non-store-operated channels predominantly carrying Na⁺ and activated by Gα_{q}- and/or Gαᵢ-coupled receptors with a complex U-shaped current-voltage relationship. Thus, individual TRPC4-containing channels contribute to agonist-induced Ca²⁺ entry directly or indirectly via depolarization and activation of voltage-gated Ca²⁺ channels. The differences in channel properties may arise from variations in the composition of the channel complexes, in the specific regulatory pathways in the corresponding cell system, and/or in the expression pattern of interaction partners which comprise other TRPC proteins to form heteromultimeric channels.

### D. Compositions and Pharmaceutical Formulations

Disclosed herein is a composition comprising an angiotensin II receptor antagonist. Disclosed herein is a composition comprising a TRPC4 antagonist. Disclosed herein is a composition comprising an angiotensin II receptor antagonist and a TRPC4 antagonist.

Disclosed herein is a composition comprising a therapeutically effective amount of an angiotensin II receptor antagonist. Disclosed herein is a composition comprising a therapeutically effective amount of a TRPC4 antagonist. Disclosed herein is a composition comprising a therapeutically effective amount of an angiotensin II receptor antagonist and a therapeutically effective amount of a TRPC4 antagonist.

Disclosed herein is a pharmaceutical formulation comprising an angiotensin II receptor antagonist one or more pharmaceutically acceptable carriers and/or excipients. Disclosed herein is a pharmaceutical formulation comprising a TRPC4 antagonist one or more pharmaceutically acceptable carriers and/or excipients. Disclosed herein is a pharmaceutical formulation comprising an angiotensin II receptor antagonist and a TRPC4 antagonist one or more pharmaceutically acceptable carriers and/or excipients.

Disclosed herein is a pharmaceutical formulation comprising a therapeutically effective amount of an angiotensin II receptor antagonist one or more pharmaceutically acceptable carriers and/or excipients. Disclosed herein is a pharmaceutical formulation comprising a therapeutically effective amount of a TRPC4 antagonist one or more pharmaceutically acceptable carriers and/or excipients. Disclosed herein is a pharmaceutical formulation comprising a therapeutically effective amount of an angiotensin II receptor antagonist and a therapeutically effective amount of a TRPC4 antagonist one or more pharmaceutically acceptable carriers and/or excipients. Carriers and excipients are discussed supra.

In an aspect, a disclosed angiotensin II receptor antagonist can comprise azilsartan or azilsartan medoxomil, candesartan, eprosartan, irbesartan, losartan, olmesartan, saprisartan, telmisartan, valsartan, any pharmaceutical salt thereof, or any combination thereof. In an aspect, a disclosed angiotensin II receptor can comprise losartan. Angiotensin II receptor antagonists are known to the art. In an aspect a disclosed TRPC4 antagonist can comprise ML204, HC-070, or the combination thereof. In an aspect, a disclosed TRPC4 antagonist can comprise any antagonist identified using OSPREY or identified pursuant to Example 3.

In an aspect, a disclosed composition or a disclosed pharmaceutical formulation can be used in a disclosed method. For example, in an aspect, a disclosed composition or a disclosed pharmaceutical formulation can be used in a disclosed method of treating and/or preventing hereditary aortopathy. In an aspect, a disclosed composition or a disclosed pharmaceutical formulation can be used in a disclosed method of treating and/or preventing an aortic aneurysm. In an aspect, a disclosed composition or a disclosed pharmaceutical formulation can be used in a disclosed method of reducing the need for surgical intervention. In an aspect, a disclosed composition or a disclosed pharmaceutical formulation can be used in a disclosed method of slowing and/or inhibiting disease progression. In an aspect, a disclosed composition or a disclosed pharmaceutical formulation can be used in a disclosed method of identifying the risk of aortic aneurysm and/or aortic dissection. In an aspect, a disclosed composition or a disclosed pharmaceutical formulation can be used in a disclosed method of extending and/or improving the life expectancy of a subject (for example, a subject having MFS).

In an aspect, a disclosed composition or a disclosed pharmaceutical formulation can slow and/or prevent the progression of an existing hereditary aortopathy, can prevent a subject from developing an aortopathy, can prevent an existing aortic aneurysm from developing and/or progressing, can reduce and/or minimize the risk of aortic aneurysm and/or aortic dissection, or any combination thereof.

In an aspect, a disclosed composition or disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof.

### E. Kits

Disclosed herein is a kit for use in a disclosed method. Disclosed herein is a kit for use in a disclosed method of treating and/or preventing hereditary aortopathy. Disclosed herein is a kit for use in a disclosed method of treating and/or preventing an aortic aneurysm. Disclosed herein is a kit for use in a disclosed method of reducing the need for surgical intervention. Disclosed herein is a kit for use in a disclosed method of slowing and/or inhibiting disease progression. Disclosed herein is a kit for use in a disclosed method of identifying the risk of aortic aneurysm and/or aortic dissection.

In an aspect, a disclosed kit can comprise any combination of reagents, compositions, tools, instruments, and containers for use—as a means of example—in obtaining a biosample from a subject, preparing a proteomic and/or genomic profile of a subject's biosample, administering a disclosed angiotensin II receptor antagonist, the amount of a disclosed TRPC4 antagonist, or a disclosed composition comprising an angiotensin II receptor antagonist and a TRPC4 antagonist, measuring the subject's hemodynamics, measuring the size of the subject's aorta, or any combination thereof.

In an aspect, a disclosed kit can comprise at least two components constituting the kit. Together, the components constitute a functional unit for a given purpose (such as, for example, administering a disclosed angiotensin II receptor antagonist, a disclosed TRPC4 antagonist, or a disclosed composition comprising an angiotensin II receptor antagonist and a TRPC4 antagonist. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

In an aspect, a kit for use in one or more disclosed methods can comprise (i) one or more disclosed reagents, compositions, tools, instruments, containers, or any combination thereof, and (ii) a label or package insert with instructions for use. In an aspect, suitable containers include, for example, bottles, vials, syringes, blister pack, etc. The containers can be formed from a variety of materials such as glass or plastic. The container can hold one or more disclosed reagents, compositions, tools, instruments, containers, or any combination thereof, and can have a sterile access port (for example the container may be an intravenous solution bag or a vial having a stopper pierceable by a hypodermic injection needle). The label or package insert can indicate that one or more disclosed reagents, compositions, tools, instruments, containers, or any combination thereof can be used in a disclosed method of treating and/or preventing hereditary aortopathy, a disclosed method of treating and/or preventing an aortic aneurysm, a disclosed method of reducing the need for surgical intervention, a disclosed method of slowing and/or inhibiting disease progression, a disclosed method of identifying the risk of aortic aneurysm and/or aortic dissection, or any combination thereof. In an aspect, a disclosed kit can comprise additional components necessary for administration such as, for example, other buffers, diluents, filters, needles, and syringes.

In an aspect, a disclosed kit can comprise one or more units of a therapeutically effective dose of a disclosed TRPC4 antagonist. In an aspect, a disclosed dose of TRPC4 antagonist can comprise about 0.01 mg/kg/day to about 200 mg/kg/day. In an aspect, a therapeutically effective dose of a disclosed TRPC4 antagonist can comprise about 1 mg/kg/day to about 20 mg/kg/day. In an aspect, a therapeutically effective dose of a disclosed TRPC4 antagonist can comprise about 50 mg/kg/day to about 150 mg/kg/day. In an aspect, a therapeutically effective dose of a disclosed TRPC4 antagonist can comprise about 100 mg/kg/day to about 200 mg/kg/day. In an aspect, a therapeutically effective dose of a disclosed TRPC4 antagonist can comprise about 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day.

In an aspect, a disclosed kit can comprise one or more units of a therapeutically effective dose of a disclosed TRPC4 antagonist. In an aspect, a disclosed dose of TRPC4 antagonist can comprise about 10 mg/kg/day. In an aspect, a therapeutically effective dose of the disclosed angiotensin II receptor antagonist can comprise about 0.01 mg/kg/day to about 200 mg/kg/day. In an aspect, a therapeutically effective dose of the disclosed angiotensin II receptor antagonist can comprise about 50 mg/kg/day to about 150 mg/kg/day. In an aspect, a therapeutically effective dose of the disclosed angiotensin II receptor antagonist can comprise about 100 mg/kg/day to about 200 mg/kg/day. In an aspect, a therapeutically effective dose of the disclosed angiotensin II receptor antagonist can comprise about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day.

In an aspect, a disclosed kit can further comprise agents, agonists, antagonists, and/or small molecules for increasing and/or decreasing the expression level of one or more aspects of the signaling cascade presented in FIG. 9.

### F. Methods of Treating and/or Preventing Hereditary Aortopathy

Disclosed herein is a method of treating and/or preventing hereditary aortopathy, the method comprising administering to a subject in need thereof a therapeutically effective amount of an angiotensin II receptor antagonist; and administering to the subject a therapeutically effective amount of a TRPC4 antagonist, wherein an existing hereditary aortopathy does not progress.

Disclosed herein is a method of preventing hereditary aortopathy, the method comprising administering to a subject at risk of developing a hereditary aortopathy a therapeutically effective amount of an angiotensin II receptor antagonist; and administering to the subject a therapeutically effective amount of a TRPC4 antagonist, wherein the subject does not develop an aortopathy.

Disclosed herein is a method of treating hereditary aortopathy, the method comprising administering to a subject in need thereof a composition comprising a therapeutically effective amount of an angiotensin II receptor antagonist and a therapeutically effective amount of a TRPC4 antagonist, wherein an existing hereditary aortopathy does not progress.

Disclosed herein is a method of preventing hereditary aortopathy, the method comprising administering to a subject at risk of developing a hereditary aortopathy a composition comprising a therapeutically effective amount of an angiotensin II receptor antagonist and a therapeutically effective amount of a TRPC4 antagonist, wherein the subject does not develop an aortopathy.

In an aspect, a disclosed composition comprising a therapeutically effective amount of an angiotensin II receptor antagonist and a therapeutically effective amount of a TRPC4 antagonist can comprise one or more pharmaceutically acceptable carriers and/or excipients. Pharmaceutically acceptable carriers and/or excipients are known to the skilled person in the art and are discussed supra.

In an aspect, a disclosed angiotensin II receptor antagonist can be administered prior to the TRPC4 antagonist, concurrently with the TRPC4 antagonist, or after the TRPC4 antagonist. In an aspect, the timing of administration of a disclosed TRPC4 antagonist can be modified.

In an aspect, a disclosed angiotensin II receptor antagonist can comprise azilsartan or azilsartan medoxomil, candesartan, eprosartan, irbesartan, losartan, olmesartan, saprisartan, telmisartan, valsartan, any pharmaceutical salt thereof, or any combination thereof. In an aspect, a disclosed angiotensin II receptor can comprise losartan. Angiotensin II receptor antagonists are known to the art.

In an aspect a disclosed TRPC4 antagonist can comprise ML204, HC-070, or the combination thereof. In an aspect, a disclosed TRPC4 antagonist can comprise any antagonist identified using OSPREY or identified pursuant to Example 3. TRPC4 is known to the art. For example, in an aspect, TRPC4 can comprise the nucleotide sequence set forth in SEQ ID NO:06 or a fragment thereof. In an aspect, the cDNA for TRPC4 can comprise the sequence set forth in SEQ ID NO:07 or a fragment thereof. In an aspect, a disclosed encoded TRPC4 can comprise the sequence set forth in SEQ ID NO:04 or SEQ ID NO:05 or a fragment thereof. Similarly, in an aspect, Trpc4 can comprise the nucleotide sequence set forth in SEQ ID NO:12 or a fragment thereof. In an aspect, the cDNA for Trpc4 can comprise the sequence set forth in SEQ ID NO:13 or a fragment thereof. In an aspect, a disclosed encoded TRPC4 can comprise the sequence set forth in SEQ ID NO:11 or a fragment thereof.

In an aspect a disclosed TRPC4 antagonist can comprise a ML204. In an aspect, ML204 is 4-Methyl-2-(piperidin-1-yl) quinoline (PubChem CID 230710) and comprises the molecular formula C₁₅H₁₈N₂. The structure of ML204 is below:

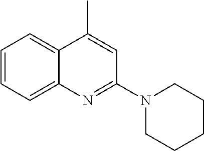 [SMILES: Cc1cc(N2CCCCC2)nc2ccccc12]

In an aspect a disclosed TRPC4 antagonist can comprise a HC-070. In an aspect, HC-070 is 8-(3-chlorophenoxy)-7-[(4-chlorophenyl)methyl]-1-(3-hydroxypropyl)-3-methylpurine-2,6-dione (PubChem CID 85473309) and comprises the molecular formula C₂₂H₂₀Cl₂N₄O₄. The structure of HC-070 is below:

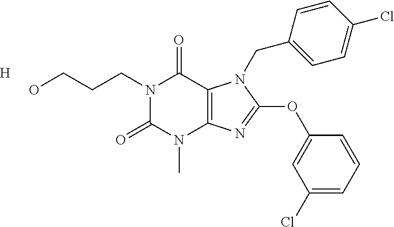

In an aspect, a disclosed method can comprise validating and/or characterizing the pharmacokinetic profile of a disclosed TRPC4 antagonist identified using OSPREY. In an aspect, validating and/or characterizing a disclosed TRPC4 antagonist can comprise using an animal model such as the disclosed aMFS mouse model.

In an aspect, a disclosed method of treating and/or preventing hereditary aortopathy can further comprise surgically repairing the aortic aneurysm and/or repairing the aortic dissection. In an aspect, surgical intervention can comprise endovascular aortic aneurysm repair (EVAR). In an aspect, if the diameter of the subject's aorta is about 50 millimeters, then surgical intervention is required. In an aspect, surgically repairing the aortic aneurysm and/or repairing the aortic dissection can comprise replacing the subject's aortic valve, the subject's aortic root, or the combination thereof.

In an aspect, a disclosed subject can have a dominant negative mutation in the FBN1 gene. In an aspect, a disclosed subject can have a haploinsufficient mutation in the FBN1 gene. FBN1 is known to the art. For example, in an aspect, FBN1 can comprise the nucleotide sequence set forth in SEQ ID NO:02 or a fragment thereof. In an aspect, the cDNA for FBN1 can comprise the sequence set forth in SEQ ID NO:03 or a fragment thereof. In an aspect, a disclosed encoded FBN1 can comprise the sequence set forth in SEQ ID NO:01 or a fragment thereof. Similarly, in an aspect, FBN1 can comprise the nucleotide sequence set forth in SEQ ID NO:09 or a fragment thereof. In an aspect, the cDNA for FBN1 can comprise the sequence set forth in SEQ ID NO:10 or a fragment thereof. In an aspect, a disclosed encoded FBN1 can comprise the sequence set forth in SEQ ID NO:08 or a fragment thereof.

In an aspect, a disclosed subject can have one or more genomic aberrations in the fibrillin-1 (FBN1) gene. Genomic aberrations can comprise mutations, rearrangements, insertions, deletion, amplifications, or any combination thereof. In an aspect of a disclosed method, a disclosed subject in need thereof can be a human being. In an aspect, a human being can be a neonate, a child, an adolescent, or an adult. In an aspect, a disclosed subject can be diagnosed with or at risk of having Marfan syndrome. In an aspect, a disclosed subject is seeking medical intervention and/or treatment.

In an aspect, a disclosed method of treating and/or preventing hereditary aortopathy can further comprise measuring aortic root diameter, measuring aortic root cross-sectional area, measuring left ventricular volume, measuring lung architecture, measuring the degree of kyphosis, or any combination thereof.

In an aspect, a disclosed method of treating and/or preventing hereditary aortopathy can comprise obtaining a biosample from the subject. In an aspect, a disclosed method of treating and/or preventing hereditary aortopathy can comprise measuring the level of one or more biomarkers in a biosample obtained from the subject. In an aspect, disclosed biomarkers can comprise circTGFB2R, miR-29a, KLF4, or any combination thereof.

In an aspect of a disclosed method of treating and/or preventing hereditary aortopathy, if the level of circTGFB2R in the subject's biosample is similar to the reference level of circTGFB2R, then the risk of the subject developing an aortic aneurysm and/or experiencing an aortic dissection is minimized. In an aspect of a disclosed method of treating and/or preventing hereditary aortopathy, if the level of circTGFB2R in the subject's biosample is less than the reference level of circTGFB2R, then the risk of the subject developing an aortic aneurysm and/or experiencing an aortic dissection is increased. In an aspect of a disclosed method of treating and/or preventing hereditary aortopathy, if the level of MiR-29a in the subject's biosample is similar to the reference level of MiR-29a, then the risk of the subject developing an aortic aneurysm and/or experiencing an aortic dissection is minimized. In an aspect of a disclosed method of treating and/or preventing hereditary aortopathy, if the level of MiR-29a in the subject's biosample is greater than the reference level of MiR-29a, then the risk of the subject developing an aortic aneurysm and/or experiencing an aortic dissection is increased. In an aspect of a disclosed method of treating and/or preventing hereditary aortopathy, if the level of circTGFB2R in the subject's biosample is similar to the reference level of circTGFB2R and the level of MiR-29a is greater than the reference level of MiR-29, then the risk of the subject developing an aortic aneurysm and/or experiencing an aortic dissection is minimized. In an aspect of a disclosed method of treating and/or preventing hereditary aortopathy, if the level of KLF4 in the subject's biosample is greater than the reference level of KLF4, then the risk of the subject developing an aortic aneurysm and/or experiencing an aortic dissection is increased. In an aspect, a disclosed method can further comprise increasing the level of and/or expression of circTGFB2R.

In an aspect, a disclosed phenotype switch vascular smooth muscle cells (VSMCs) can be prevented. In an aspect, a disclosed phenotype switch in VSMCs can comprise a switch from a contractile phenotype of to a synthetic phenotype. In an aspect, a disclosed contractile phenotype can comprise expression of one or more differentiation markers. In an aspect, disclosed differentiation markers can comprise SM22α and/or α-SMA. In an aspect, a disclosed synthetic phenotype can comprise expression of one or more synthetic markers. In an aspect, disclosed synthetic markers can comprise osteopontin (OPN) and/or matrix Gla protein (MGP).

In an aspect of a disclosed method of treating and/or preventing hereditary aortopathy, administering a composition comprising a therapeutically effective amount of an angiotensin II receptor antagonist can inhibit the expression of one or more synthetic markers. In an aspect of a disclosed method of treating and/or preventing hereditary aortopathy, administering a composition comprising a therapeutically effective amount of a TRPC4 antagonist can inhibit the expression of one or more synthetic markers. In an aspect of a disclosed method of treating and/or preventing hereditary aortopathy, administering a composition comprising a therapeutically effective amount of an angiotensin II receptor antagonist and a therapeutically effective amount of a TRPC4 antagonist can inhibit the expression of one or more synthetic markers. In an aspect of a disclosed method of treating and/or preventing hereditary aortopathy, administering a therapeutically effective amount of an angiotensin II receptor antagonist or a therapeutically effective amount of a TRPC4 antagonist can inhibit the expression of one or more synthetic markers.

In an aspect, a disclosed method of treating and/or preventing hereditary aortopathy can comprise measuring the expression level of one or more markers of aortopathy in a biosample obtained from the subject. In an aspect, disclosed markers of aortopathy can comprise Smad2, Smad3, JNK1, ERK1/2, ERK5, MEK1, ROCK1, p38, MMP2, MMP9, MMP17, TRPC4, or any combination thereof. In an aspect, a disclosed expression level can comprise the level of protein expression, the level of miRNA expression, the level of mRNA expression, or the combination thereof. Methods and techniques for determining the expression and/or activity level of a disclosed biomarker, protein, peptide, microRNA (mi-R), messenger RNA (mRNA), DNA, or any combination thereof are known to the art and are disclosed herein.

In an aspect, a biosample can comprise a tissue or tissues, a cell or cells, a biopsied tissue or tissues, a biopsied cell or cells, blood, lymph, CFS, serum, plasma, urine, saliva, mucus, tears, or a combination thereof.

In an aspect, a disclosed method of treating and/or preventing hereditary aortopathy can further comprise generating one or more times a metabolic or genomic profile for the subject.

In an aspect, a disclosed method of treating and/or preventing hereditary aortopathy can further comprise generating one or more times a proteomic profile for the subject. For example, a proteomic profile can be generated for all or for some of the biosamples obtained from the subject. In an aspect, a proteomic profile can be generated prior to a disclosed administering step, after a disclosed administering step, or both.

As used herein, proteomic profile can mean the combination of proteins found in a subject's biosample. The proteomic profile is a collection of measurements, such as but not limited to a quantity or concentration, for individual proteins taken from a subject's biosample. Techniques to determine the levels of individual components of the proteomic profile from biosamples are well known to the skilled technician and include, but are not limited to, mass spectrometry, ultra-performance liquid chromatography (UPLC), high-performance liquid chromatography (HPLC), mass spectrometry in conjunction with UPLC, LC/MS/MS, ELISA, and Western blots.

In an aspect, a disclosed method of treating and/or preventing hereditary aortopathy can further comprise generating one or more times a miRNA profile for the subject. For example, a miRNA profile can be generated for all or for some of the biosamples obtained from the subject. In an aspect, a miRNA profile can be generated prior to a disclosed administering step, after a disclosed administering step, or both. As used herein, miRNA profile can mean the combination of miRNAs found in a subject's biosample. The miRNA profile is a collection of measurements, such as but not limited to a quantity or concentration, for individual miRNAs taken from a subject's biosample. Techniques to determine the levels of individual components of the miRNA profile from biosamples are well known to the skilled technician and include, but are not limited to, RNAseq and RT-qPCR.

The assessment of the levels of the individual components of the proteomic and/or miRNA profile can be expressed as absolute or relative values and may or may not be expressed in relation to another component, a standard, an internal standard, or another molecule of compound known to be in the sample. If the levels are assessed as relative to a standard or internal standard, then the standard can be added to the test sample prior to, during, or after sample processing.

The level of differential expression of proteins and/or miRNAs in a biosample when compared to a reference biosample (or any other biosample) can vary. For example, the level of any one or more differentially expressed proteins (such as, for example, circTGFB2R) and/or miRNAs (such as, for example miR-29a) in a biosample can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold lower than that of a reference biosample. Or the levels of any one or more differentially expressed proteins and/or miRNAs can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold higher than that of a reference biosample. In an aspect, the number of “times” the level of one or more differentially expressed proteins and/or miRNAs is lower or higher than that of a reference level can be a relative or an absolute number of times. Or, in an aspect, the level of the proteins and/or miRNAs can be normalized to a standard and these normalized levels can then be compared to one another to determine whether the differentially expressed proteins and/or miRNAs is lower or higher.

In an aspect, administering can comprise titrating a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects. In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a therapeutically effective dose of the disclosed angiotensin II receptor antagonist can comprise about 0.01 mg/kg/day to about 200 mg/kg/day. In an aspect, a therapeutically effective dose of the disclosed angiotensin II receptor antagonist can comprise about 50 mg/kg/day to about 150 mg/kg/day. In an aspect, a therapeutically effective dose of the disclosed angiotensin II receptor antagonist can comprise about 100 mg/kg/day to about 200 mg/kg/day. In an aspect, a therapeutically effective dose of the disclosed angiotensin II receptor antagonist can comprise about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day.

In an aspect, a therapeutically effective dose of a disclosed TRPC4 antagonist can comprise about 0.01 mg/kg/day to about 200 mg/kg/day. In an aspect, a therapeutically effective dose of a disclosed TRPC4 antagonist can comprise about 1 mg/kg/day to about 20 mg/kg/day. In an aspect, a therapeutically effective dose of a disclosed TRPC4 antagonist can comprise about 50 mg/kg/day to about 150 mg/kg/day. In an aspect, a therapeutically effective dose of a disclosed TRPC4 antagonist can comprise about 100 mg/kg/day to about 200 mg/kg/day. In an aspect, a therapeutically effective dose of a disclosed TRPC4 antagonist can comprise about 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day. In an aspect, a therapeutically effective dose of a disclosed TRPC4 antagonist can comprise about 10 mg/kg/day.

In an aspect, a disclosed method of treating and/or preventing hereditary aortopathy can further comprise increasing and/or decreasing the expression level of one or more aspects of the signaling cascade presented in FIG. 9.

In an aspect, a disclosed method of treating and/or preventing hereditary aortopathy can further comprise (i) treating and/or preventing an aortic aneurysm, (ii) reducing the need for surgical intervention, (iii) slowing and/or inhibiting disease progression, (iv) identifying the risk of aortic aneurysm and/or aortic dissection, or (v) any combination thereof.

In an aspect, a disclosed method can comprise repeating an administering step one or more times. For example, a disclosed TRPC4 antagonist can be administered one or more times. In an aspect, a disclosed angiotensin II receptor antagonist can be administered one or more times. In an aspect, a disclosed composition or pharmaceutical composition comprising a disclosed TRPC4 antagonist and/or a disclosed angiotensin II receptor antagonist can be administered one or more times. In an aspect, administering of any disclosed antagonist, disclosed composition, and/or disclosed pharmaceutical formulation can comprise intravenous administration or oral administration.

In an aspect, the number of surgeries required by the subject in need thereof can be reduced by 1 surgery, 2 surgeries, 3 surgeries, 4 surgeries, 5 surgeries, or more than 5 surgeries than a subject not being subjected to or exposed to a disclosed method. For example, a subject in thereof can be subjected to 1 fewer surgery, 2 fewer surgeries, 3 fewer surgeries, 4 fewer surgeries, 5 fewer surgeries, or more than a subject not being subjected to or exposed to a disclosed method. In an aspect, the number of surgeries required by the subject in need thereof can be reduced by 10%, 20%, 30%, 40%, 50%, 60%, 70%, 80%, 90%, or more than 90% when compared to a subject not being subjected to or exposed to a disclosed method. In an aspect, the number of surgeries required by the subject in need thereof can be reduced by 10%-30%, 30%-60%, 60%-90%, or more than 90% when compared to a subject not being subjected to or exposed to a disclosed method. In an aspect, a disclosed subject can be subjected to a disclosed method and not require additional surgeries.

In an aspect of a disclosed method, the number of surgeries required by the subject in need thereof can be reduced when compared to the number of surgeries required by a subject not receiving a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a disclosed method of treating and/or preventing hereditary aortopathy can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both. In an aspect, modifying the treating step can comprise changing the amount of a disclosed angiotensin II receptor antagonist, the amount of a disclosed TRPC4 antagonist, or the amount of a disclosed composition comprising an angiotensin II receptor antagonist and a TRPC4 antagonist administered to the subject, changing the frequency of administration of a disclosed angiotensin II receptor antagonist, the amount of a disclosed TRPC4 antagonist, or the amount of a disclosed composition comprising an angiotensin II receptor antagonist and a TRPC4 antagonist, changing the route of administration of a disclosed angiotensin II receptor antagonist, the amount of a disclosed TRPC4 antagonist, or the amount of a disclosed composition comprising an angiotensin II receptor antagonist and a TRPC4 antagonist, or any combination thereof. In an aspect, modifying the administering step can comprise changing the amount of a disclosed angiotensin II receptor and/or a disclosed TRPC4 antagonist, a disclosed composition comprising a disclosed angiotensin II receptor and/or a disclosed TRPC4 antagonist.

In an aspect, a disclosed method of treating and/or preventing hereditary aortopathy can comprise improving and/or increasing the life expectancy of a disclosed subject or a disclosed patient. In an aspect of a disclosed method, the life expectancy of the recipient or subject in need thereof is increased. In an aspect, life expectancy can be increased by 5-10 years, by 10-15 years, by 15-20 years, by 20-25 years, by 25-30 years, by 30-35 years, by 35-40 years, or more than 40 years when compared to a subject not being subjected to or exposed to a disclosed method.

In an aspect, a disclosed method of treating and/or preventing hereditary aortopathy can further comprise improving the subject's hemodynamics. Hemodynamics are known to the skill in the art. In an aspect, hemodynamics can comprise blow flow in the aortic root, sinuses, coronary orifices, or any combination thereof.

In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects.

In an aspect, a disclosed method of treating and/or preventing hereditary aortopathy can further comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof in a specific or disclosed subject. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects for a specific or disclosed subject.

In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, the growth of an aneurysm can be measured using echocardiography, imaging techniques (e.g., MRI and/or computed tomography), abdominal or pelvic ultrasonography, or any combination thereof. In an aspect, aortic aneurysm size can be a critical determinant of the need for intervention, yet the maximal diameter can often vary depending on the modality and method of measurement. In an aspect, a CT scan of a disclosed subject or disclosed patient can comprise using a three-dimensional reconstruction software. In an aspect, maximal aortic diameter can be recorded in the anteroposterior (CT-AP) plane, the maximal ellipse (CT-ME), perpendicular to the maximal ellipse (CT-PME), or perpendicular to the centerline of flow (CT-PCLF), or any combination thereof. In an aspect, a disclosed aneurysm can be analyzed according to the principles of Bland and Altman.

In an aspect, a disclosed method of treating and/or preventing hereditary aortopathy can further comprise administering to the subject (i) one or more active agents, (ii) biologically active agents, (iii) one or more pharmaceutically active agents, (iv) one or more immune-based therapeutic agents, (v) one or more clinically approved agents, or (vi) a combination thereof.

### G. Methods of Treating and/or Preventing Aortic Aneurysm

Disclosed herein is a method of treating an aortic aneurysm, the method comprising administering to a subject in need thereof a therapeutically effective amount of an angiotensin II receptor antagonist; and administering to the subject a therapeutically effective amount of a TRPC4 antagonist, wherein an existing an aortic aneurysm does not progress.

Disclosed herein is a method of treating an aortic aneurysm, the method comprising administering to a subject at risk of developing a hereditary aortopathy a therapeutically effective amount of an angiotensin II receptor antagonist; and administering to the subject a therapeutically effective amount of a TRPC4 antagonist, wherein the subject does not develop an aortic aneurysm.

Disclosed herein is a method of treating an aortic aneurysm, the method comprising administering to a subject in need thereof a composition comprising a therapeutically effective amount of an angiotensin II receptor antagonist and a therapeutically effective amount of a TRPC4 antagonist, wherein an existing an aortic aneurysm does not progress.

Disclosed herein is a method of treating an aortic aneurysm, the method comprising administering to a subject at risk of developing a hereditary aortopathy a composition comprising a therapeutically effective amount of an angiotensin II receptor antagonist and a therapeutically effective amount of a TRPC4 antagonist, wherein the subject does not develop an aortic aneurysm.

In an aspect, a disclosed composition comprising a therapeutically effective amount of an angiotensin II receptor antagonist and a therapeutically effective amount of a TRPC4 antagonist can comprise one or more pharmaceutically acceptable carriers and/or excipients.

Pharmaceutically acceptable carriers and/or excipients are known to the skilled person in the art and are discussed supra.

In an aspect, a disclosed angiotensin II receptor antagonist can be administered prior to the TRPC4 antagonist, concurrently with the TRPC4 antagonist, or after the TRPC4 antagonist. In an aspect, the timing of administration of a disclosed TRPC4 antagonist can be modified.

In an aspect, a disclosed angiotensin II receptor antagonist can comprise azilsartan or azilsartan medoxomil, candesartan, eprosartan, irbesartan, losartan, olmesartan, saprisartan, telmisartan, valsartan, any pharmaceutical salt thereof, or any combination thereof. In an aspect, a disclosed angiotensin II receptor can comprise losartan. Angiotensin II receptor antagonists are known to the art.

In an aspect a disclosed TRPC4 antagonist can comprise ML204, HC-070, or the combination thereof. In an aspect, a disclosed TRPC4 antagonist can comprise any antagonist identified using OSPREY or identified pursuant to Example 3. TRPC4 is known to the art. For example, in an aspect, TRPC4 can comprise the nucleotide sequence set forth in SEQ ID NO:06 or a fragment thereof. In an aspect, the cDNA for TRPC4 can comprise the sequence set forth in SEQ ID NO:07 or a fragment thereof. In an aspect, a disclosed encoded TRPC4 can comprise the sequence set forth in SEQ ID NO:04 or SEQ ID NO:05 or a fragment thereof. Similarly, in an aspect, Trpc4 can comprise the nucleotide sequence set forth in SEQ ID NO:12 or a fragment thereof. In an aspect, the cDNA for Trpc4 can comprise the sequence set forth in SEQ ID NO:13 or a fragment thereof. In an aspect, a disclosed encoded TRPC4 can comprise the sequence set forth in SEQ ID NO:11 or a fragment thereof.

In an aspect a disclosed TRPC4 antagonist can comprise a ML204. In an aspect, ML204 is 4-Methyl-2-(piperidin-1-yl) quinoline (PubChem CID 230710) and comprises the molecular formula C₁₅H₁₈N₂. The structure of ML204 is provided supra.

In an aspect a disclosed TRPC4 antagonist can comprise a HC-070. In an aspect, HC-070 is 8-(3-chlorophenoxy)-7-[(4-chlorophenyl)methyl]-1-(3-hydroxypropyl)-3-methylpurine-2,6-dione (PubChem CID 85473309) and comprises the molecular formula C₂₂H₂₀Cl₂N₄O₄. The structure of HC-070 is provided supra.

In an aspect, a disclosed method can comprise validating and/or characterizing the pharmacokinetic profile of a disclosed TRPC4 antagonist identified using OSPREY. In an aspect, validating and/or characterizing a disclosed TRPC4 antagonist can comprise using an animal model such as the disclosed aMFS mouse model.

In an aspect, a disclosed method of treating an aortic aneurysm can further comprise surgically repairing the aortic aneurysm and/or repairing the aortic dissection. In an aspect, surgical intervention can comprise endovascular aortic aneurysm repair (EVAR). In an aspect, if the diameter of the subject's aorta is about 50 millimeters, then surgical intervention is required. In an aspect, surgically repairing the aortic aneurysm and/or repairing the aortic dissection can comprise replacing the subject's aortic valve, the subject's aortic root, or the combination thereof.

In an aspect, a disclosed subject can have a dominant negative mutation in the FBN1 gene. In an aspect, a disclosed subject can have a haploinsufficient mutation in the FBN1 gene. FBN1 is known to the art. For example, in an aspect, FBN1 can comprise the nucleotide sequence set forth in SEQ ID NO:02 or a fragment thereof. In an aspect, the cDNA for FBN1 can comprise the sequence set forth in SEQ ID NO:03 or a fragment thereof. In an aspect, a disclosed encoded FBN1 can comprise the sequence set forth in SEQ ID NO:01 or a fragment thereof. Similarly, in an aspect, FBN1 can comprise the nucleotide sequence set forth in SEQ ID NO:09 or a fragment thereof. In an aspect, the cDNA for FBN1 can comprise the sequence set forth in SEQ ID NO:10 or a fragment thereof. In an aspect, a disclosed encoded FBN1 can comprise the sequence set forth in SEQ ID NO:08 or a fragment thereof.

In an aspect, a disclosed subject can have one or more genomic aberrations in the fibrillin-1 (FBN1) gene. Genomic aberrations can comprise mutations, rearrangements, insertions, deletion, amplifications, or any combination thereof. In an aspect of a disclosed method of treating an aortic aneurysm, a disclosed subject in need thereof can be a human being. In an aspect, a human being can be a neonate, a child, an adolescent, or an adult. In an aspect, a disclosed subject can be diagnosed with or at risk of having Marfan syndrome. In an aspect, a disclosed subject is seeking medical intervention and/or treatment.

In an aspect, a disclosed method of treating an aortic aneurysm can further comprise measuring aortic root diameter, measuring aortic root cross-sectional area, measuring left ventricular volume, measuring lung architecture, measuring the degree of kyphosis, or any combination thereof.

In an aspect, a disclosed method of treating an aortic aneurysm can comprise obtaining a biosample from the subject. In an aspect, a disclosed method of treating an aortic aneurysm can comprise measuring the level of one or more biomarkers in a biosample obtained from the subject. In an aspect, disclosed biomarkers can comprise circTGFB2R, miR-29a, KLF4, or any combination thereof.

In an aspect of a disclosed method of treating an aortic aneurysm if the level of circTGFB2R in the subject's biosample is similar to the reference level of circTGFB2R, then the risk of the subject developing an aortic aneurysm and/or experiencing an aortic dissection is minimized. In an aspect of a disclosed method of treating an aortic aneurysm, if the level of circTGFB2R in the subject's biosample is less than the reference level of circTGFB2R, then the risk of the subject developing an aortic aneurysm and/or experiencing an aortic dissection is increased. In an aspect of a disclosed method of treating an aortic aneurysm, if the level of MiR-29a in the subject's biosample is similar to the reference level of MiR-29a, then the risk of the subject developing an aortic aneurysm and/or experiencing an aortic dissection is minimized. In an aspect of a disclosed method of treating an aortic aneurysm, if the level of MiR-29a in the subject's biosample is greater than the reference level of MiR-29a, then the risk of the subject developing an aortic aneurysm and/or experiencing an aortic dissection is increased. In an aspect of a disclosed method of treating an aortic aneurysm, if the level of circTGFB2R in the subject's biosample is similar to the reference level of circTGFB2R and the level of MiR-29a is greater than the reference level of MiR-29, then the risk of the subject developing an aortic aneurysm and/or experiencing an aortic dissection is minimized. In an aspect of a disclosed method of treating an aortic aneurysm, if the level of KLF4 in the subject's biosample is greater than the reference level of KLF4, then the risk of the subject developing an aortic aneurysm and/or experiencing an aortic dissection is increased. In an aspect, a disclosed method can further comprise increasing the level of and/or expression of circTGFB2R.

In an aspect, a disclosed phenotype switch vascular smooth muscle cells (VSMCs) can be prevented. In an aspect, a disclosed phenotype switch in VSMCs can comprise a switch from a contractile phenotype of to a synthetic phenotype. In an aspect, a disclosed contractile phenotype can comprise expression of one or more differentiation markers. In an aspect, disclosed differentiation markers can comprise SM22α and/or α-SMA. In an aspect, a disclosed synthetic phenotype can comprise expression of one or more synthetic markers. In an aspect, disclosed synthetic markers can comprise osteopontin (OPN) and/or matrix Gla protein (MGP).

In an aspect of a disclosed method of treating an aortic aneurysm, administering a composition comprising a therapeutically effective amount of an angiotensin II receptor antagonist can inhibit the expression of one or more synthetic markers. In an aspect of a disclosed method of treating an aortic aneurysm, administering a composition comprising a therapeutically effective amount of a TRPC4 antagonist can inhibit the expression of one or more synthetic markers. In an aspect of a disclosed method of treating an aortic aneurysm, administering a composition comprising a therapeutically effective amount of an angiotensin II receptor antagonist and a therapeutically effective amount of a TRPC4 antagonist can inhibit the expression of one or more synthetic markers. In an aspect of a disclosed method of treating an aortic aneurysm, administering a therapeutically effective amount of an angiotensin II receptor antagonist or a therapeutically effective amount of a TRPC4 antagonist can inhibit the expression of one or more synthetic markers.

In an aspect, a disclosed method of treating an aortic aneurysm can comprise measuring the expression level of one or more markers of aortopathy in a biosample obtained from the subject. In an aspect, disclosed markers of aortopathy can comprise Smad2, Smad3, JNK1, ERK1/2, ERK5, MEK1, ROCK1, p38, MMP2, MMP9, MMP17, TRPC4, or any combination thereof. In an aspect, a disclosed expression level can comprise the level of protein expression, the level of miRNA expression, the level of mRNA expression, or the combination thereof. Methods and techniques for determining the expression and/or activity level of a disclosed biomarker, protein, peptide, microRNA (mi-R), messenger RNA (mRNA), DNA, or any combination thereof are known to the art and are disclosed herein.

In an aspect, a biosample can comprise a tissue or tissues, a cell or cells, a biopsied tissue or tissues, a biopsied cell or cells, blood, lymph, CFS, serum, plasma, urine, saliva, mucus, tears, or a combination thereof.

In an aspect, a disclosed method of treating an aortic aneurysm can further comprise generating one or more times a metabolic or genomic profile for the subject.

In an aspect, a disclosed method of treating an aortic aneurysm can further comprise generating one or more times a proteomic profile for the subject. For example, a proteomic profile can be generated for all or for some of the biosamples obtained from the subject. In an aspect, a proteomic profile can be generated prior to a disclosed administering step, after a disclosed administering step, or both.

As used herein, proteomic profile can mean the combination of proteins found in a subject's biosample. The proteomic profile is a collection of measurements, such as but not limited to a quantity or concentration, for individual proteins taken from a subject's biosample. Techniques to determine the levels of individual components of the proteomic profile from biosamples are well known to the skilled technician and include, but are not limited to, mass spectrometry, ultra-performance liquid chromatography (UPLC), high-performance liquid chromatography (HPLC), mass spectrometry in conjunction with UPLC, LC/MS/MS, ELISA, and Western blots.

In an aspect, a disclosed method of treating an aortic aneurysm further comprise generating one or more times a miRNA profile for the subject. For example, a miRNA profile can be generated for all or for some of the biosamples obtained from the subject. In an aspect, a miRNA profile can be generated prior to a disclosed administering step, after a disclosed administering step, or both. As used herein, miRNA profile can mean the combination of miRNAs found in a subject's biosample. The miRNA profile is a collection of measurements, such as but not limited to a quantity or concentration, for individual miRNAs taken from a subject's biosample. Techniques to determine the levels of individual components of the miRNA profile from biosamples are well known to the skilled technician and include, but are not limited to, RNAseq and RT-qPCR.

The assessment of the levels of the individual components of the proteomic and/or miRNA profile can be expressed as absolute or relative values and may or may not be expressed in relation to another component, a standard, an internal standard, or another molecule of compound known to be in the sample. If the levels are assessed as relative to a standard or internal standard, then the standard can be added to the test sample prior to, during, or after sample processing.

The level of differential expression of proteins and/or miRNAs in a biosample when compared to a reference biosample (or any other biosample) can vary. For example, the level of any one or more differentially expressed proteins (such as, for example, circTGFB2R) and/or miRNAs (such as, for example miR-29a) in a biosample can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold lower than that of a reference biosample. Or the levels of any one or more differentially expressed proteins and/or miRNAs can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold higher than that of a reference biosample. In an aspect, the number of “times” the level of one or more differentially expressed proteins and/or miRNAs is lower or higher than that of a reference level can be a relative or an absolute number of times. Or, in an aspect, the level of the proteins and/or miRNAs can be normalized to a standard and these normalized levels can then be compared to one another to determine whether the differentially expressed proteins and/or miRNAs is lower or higher.

In an aspect, administering can comprise titrating a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects. In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a therapeutically effective dose of the disclosed angiotensin II receptor antagonist can comprise about 0.01 mg/kg/day to about 200 mg/kg/day. In an aspect, a therapeutically effective dose of the disclosed angiotensin II receptor antagonist can comprise about 50 mg/kg/day to about 150 mg/kg/day. In an aspect, a therapeutically effective dose of the disclosed angiotensin II receptor antagonist can comprise about 100 mg/kg/day to about 200 mg/kg/day. In an aspect, a therapeutically effective dose of the disclosed angiotensin II receptor antagonist can comprise about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day.

In an aspect, a therapeutically effective dose of a disclosed TRPC4 antagonist can comprise about 0.01 mg/kg/day to about 200 mg/kg/day. In an aspect, a therapeutically effective dose of a disclosed TRPC4 antagonist can comprise about 1 mg/kg/day to about 20 mg/kg/day. In an aspect, a therapeutically effective dose of a disclosed TRPC4 antagonist can comprise about 50 mg/kg/day to about 150 mg/kg/day. In an aspect, a therapeutically effective dose of a disclosed TRPC4 antagonist can comprise about 100 mg/kg/day to about 200 mg/kg/day. In an aspect, a therapeutically effective dose of a disclosed TRPC4 antagonist can comprise about 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day. In an aspect, a therapeutically effective dose of a disclosed TRPC4 antagonist can comprise about 10 mg/kg/day.

In an aspect, a disclosed method of treating an aortic aneurysm can further comprise increasing and/or decreasing the expression level of one or more aspects of the signaling cascade presented in FIG. 9.

In an aspect, a disclosed method of treating an aortic aneurysm can further comprise (i) treating and/or preventing hereditary aortopathy, (ii) reducing the need for surgical intervention, (iii) slowing and/or inhibiting disease progression, (iv) identifying the risk of aortic aneurysm and/or aortic dissection, or (v) any combination thereof.

In an aspect, a disclosed method can comprise repeating an administering step one or more times. For example, a disclosed TRPC4 antagonist can be administered one or more times. In an aspect, a disclosed angiotensin II receptor antagonist can be administered one or more times. In an aspect, a disclosed composition or pharmaceutical composition comprising a disclosed TRPC4 antagonist and/or a disclosed angiotensin II receptor antagonist can be administered one or more times. In an aspect, administering of any disclosed antagonist, disclosed composition, and/or disclosed pharmaceutical formulation can comprise intravenous administration or oral administration.

In an aspect, the number of surgeries required by the subject in need thereof can be reduced by 1 surgery, 2 surgeries, 3 surgeries, 4 surgeries, 5 surgeries, or more than 5 surgeries than a subject not being subjected to or exposed to a disclosed method. For example, a subject in thereof can be subjected to 1 fewer surgery, 2 fewer surgeries, 3 fewer surgeries, 4 fewer surgeries, 5 fewer surgeries, or more than a subject not being subjected to or exposed to a disclosed method. In an aspect, the number of surgeries required by the subject in need thereof can be reduced by 10%, 20%, 30%, 40%, 50%, 60%, 70%, 80%, 90%, or more than 90% when compared to a subject not being subjected to or exposed to a disclosed method. In an aspect, the number of surgeries required by the subject in need thereof can be reduced by 10%-30%, 30%-60%, 60%-90%, or more than 90% when compared to a subject not being subjected to or exposed to a disclosed method. In an aspect, a disclosed subject can be subjected to a disclosed method and not require additional surgeries.

In an aspect of a disclosed method, the number of surgeries required by the subject in need thereof can be reduced when compared to the number of surgeries required by a subject not receiving a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a disclosed method of treating an aortic aneurysm can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method.

In an aspect, modifying can comprise modifying the treating step, modifying the administering step, or both. In an aspect, modifying the treating step can comprise changing the amount of a disclosed angiotensin II receptor antagonist, the amount of a disclosed TRPC4 antagonist, or the amount of a disclosed composition comprising an angiotensin II receptor antagonist and a TRPC4 antagonist administered to the subject, changing the frequency of administration of a disclosed angiotensin II receptor antagonist, the amount of a disclosed TRPC4 antagonist, or the amount of a disclosed composition comprising an angiotensin II receptor antagonist and a TRPC4 antagonist, changing the route of administration of a disclosed angiotensin II receptor antagonist, the amount of a disclosed TRPC4 antagonist, or the amount of a disclosed composition comprising an angiotensin II receptor antagonist and a TRPC4 antagonist, or any combination thereof. In an aspect, modifying the administering step can comprise changing the amount of a disclosed angiotensin II receptor and/or a disclosed TRPC4 antagonist, a disclosed composition comprising a disclosed angiotensin II receptor and/or a disclosed TRPC4 antagonist.

In an aspect, a disclosed method of treating an aortic aneurysm can comprise improving and/or increasing the life expectancy of a disclosed subject or a disclosed patient. In an aspect of a disclosed method, the life expectancy of the recipient or subject in need thereof is increased. In an aspect, life expectancy can be increased by 5-10 years, by 10-15 years, by 15-20 years, by 20-25 years, by 25-30 years, by 30-35 years, by 35-40 years, or more than 40 years when compared to a subject not being subjected to or exposed to a disclosed method.

In an aspect, a disclosed method of treating an aortic aneurysm can further comprise improving the subject's hemodynamics. Hemodynamics are known to the skill in the art. In an aspect, hemodynamics can comprise blow flow in the aortic root, sinuses, coronary orifices, or any combination thereof.

In an aspect, a disclosed method of treating an aortic aneurysm an comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects.

In an aspect, a disclosed method of treating an aortic aneurysm can further comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof in a specific or disclosed subject. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects for a specific or disclosed subject.

In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, the growth of an aneurysm can be measured using echocardiography, imaging techniques (e.g., MRI and/or computed tomography), abdominal or pelvic ultrasonography, or any combination thereof. In an aspect, aortic aneurysm size can be a critical determinant of the need for intervention, yet the maximal diameter can often vary depending on the modality and method of measurement. In an aspect, a CT scan of a disclosed subject or disclosed patient can comprise using a three-dimensional reconstruction software. In an aspect, maximal aortic diameter can be recorded in the anteroposterior (CT-AP) plane, the maximal ellipse (CT-ME), perpendicular to the maximal ellipse (CT-PME), or perpendicular to the centerline of flow (CT-PCLF), or any combination thereof. In an aspect, a disclosed aneurysm can be analyzed according to the principles of Bland and Altman.

In an aspect, a disclosed method of treating and/or preventing an aortic aneurysm can further comprise administering to the subject (i) one or more active agents, (ii) biologically active agents, (iii) one or more pharmaceutically active agents, (iv) one or more immune-based therapeutic agents, (v) one or more clinically approved agents, or (vi) a combination thereof.

### H. Methods of Reducing the Need for Surgical Intervention

Disclosed herein is a method of reducing the need for surgical intervention, the method comprising administering to a subject in need thereof a therapeutically effective amount of an angiotensin II receptor antagonist; and administering to the subject a therapeutically effective amount of a TRPC4 antagonist, wherein an existing hereditary aortopathy does not progress or wherein the subject does not develop an aortic aneurysm, and wherein surgical intervention is not needed.

Disclosed herein is a method of reducing the need for surgical intervention, the method comprising administering to a subject in need thereof a composition comprising a therapeutically effective amount of an angiotensin II receptor antagonist and a therapeutically effective amount of a TRPC4 antagonist, wherein an existing hereditary aortopathy does not progress or wherein the subject does not develop an aortic aneurysm, and wherein surgical intervention is not needed.

In an aspect, a disclosed composition comprising a therapeutically effective amount of an angiotensin II receptor antagonist and a therapeutically effective amount of a TRPC4 antagonist can comprise one or more pharmaceutically acceptable carriers and/or excipients.

Pharmaceutically acceptable carriers and/or excipients are known to the skilled person in the art and are discussed supra.

In an aspect, a disclosed angiotensin II receptor antagonist can be administered prior to the TRPC4 antagonist, concurrently with the TRPC4 antagonist, or after the TRPC4 antagonist. In an aspect, the timing of administration of a disclosed TRPC4 antagonist can be modified.

In an aspect, a disclosed angiotensin II receptor antagonist can comprise azilsartan or azilsartan medoxomil, candesartan, eprosartan, irbesartan, losartan, olmesartan, saprisartan, telmisartan, valsartan, any pharmaceutical salt thereof, or any combination thereof. In an aspect, a disclosed angiotensin II receptor can comprise losartan. Angiotensin II receptor antagonists are known to the art.

In an aspect a disclosed TRPC4 antagonist can comprise ML204, HC-070, or the combination thereof. In an aspect, a disclosed TRPC4 antagonist can comprise any antagonist identified using OSPREY or identified pursuant to Example 3. TRPC4 is known to the art. For example, in an aspect, TRPC4 can comprise the nucleotide sequence set forth in SEQ ID NO:06 or a fragment thereof. In an aspect, the cDNA for TRPC4 can comprise the sequence set forth in SEQ ID NO:07 or a fragment thereof. In an aspect, a disclosed encoded TRPC4 can comprise the sequence set forth in SEQ ID NO:04 or SEQ ID NO:05 or a fragment thereof. Similarly, in an aspect, Trpc4 can comprise the nucleotide sequence set forth in SEQ ID NO:12 or a fragment thereof. In an aspect, the cDNA for Trpc4 can comprise the sequence set forth in SEQ ID NO:13 or a fragment thereof. In an aspect, a disclosed encoded TRPC4 can comprise the sequence set forth in SEQ ID NO:11 or a fragment thereof.

In an aspect a disclosed TRPC4 antagonist can comprise a ML204. In an aspect, ML204 is 4-Methyl-2-(piperidin-1-yl) quinoline (PubChem CID 230710) and comprises the molecular formula C₁₅H₁₈N₂. The structure of ML204 is provided supra.

In an aspect a disclosed TRPC4 antagonist can comprise a HC-070. In an aspect, HC-070 is 8-(3-chlorophenoxy)-7-[(4-chlorophenyl)methyl]-1-(3-hydroxypropyl)-3-methylpurine-2,6-dione (PubChem CID 85473309) and comprises the molecular formula C₂₂H₂₀Cl₂N₄O₄. The structure of HC-070 is provided supra.

In an aspect, a disclosed method can comprise validating and/or characterizing the pharmacokinetic profile of a disclosed TRPC4 antagonist identified using OSPREY. In an aspect, validating and/or characterizing a disclosed TRPC4 antagonist can comprise using an animal model such as the disclosed aMFS mouse model.

In an aspect, a disclosed method of treating and/or preventing hereditary aortopathy can further comprise surgically repairing the aortic aneurysm and/or repairing the aortic dissection. In an aspect, surgical intervention can comprise endovascular aortic aneurysm repair (EVAR). In an aspect, if the diameter of the subject's aorta is about 50 millimeters, then surgical intervention is required. In an aspect, surgically repairing the aortic aneurysm and/or repairing the aortic dissection can comprise replacing the subject's aortic valve, the subject's aortic root, or the combination thereof.

In an aspect, a disclosed subject can have a dominant negative mutation in the FBN1 gene. In an aspect, a disclosed subject can have a haploinsufficient mutation in the FBN1 gene. FBN1 is known to the art. For example, in an aspect, FBN1 can comprise the nucleotide sequence set forth in SEQ ID NO:02 or a fragment thereof. In an aspect, the cDNA for FBN1 can comprise the sequence set forth in SEQ ID NO:03 or a fragment thereof. In an aspect, a disclosed encoded FBN1 can comprise the sequence set forth in SEQ ID NO:01 or a fragment thereof. Similarly, in an aspect, FBN1 can comprise the nucleotide sequence set forth in SEQ ID NO:09 or a fragment thereof. In an aspect, the cDNA for FBN1 can comprise the sequence set forth in SEQ ID NO:10 or a fragment thereof. In an aspect, a disclosed encoded FBN1 can comprise the sequence set forth in SEQ ID NO:08 or a fragment thereof.

In an aspect, a disclosed subject can have one or more genomic aberrations in the fibrillin-1 (FBN1) gene. Genomic aberrations can comprise mutations, rearrangements, insertions, deletion, amplifications, or any combination thereof. In an aspect of a disclosed method, a disclosed subject in need thereof can be a human being. In an aspect, a human being can be a neonate, a child, an adolescent, or an adult. In an aspect, a disclosed subject can be diagnosed with or at risk of having Marfan syndrome. In an aspect, a disclosed subject is seeking medical intervention and/or treatment.

In an aspect, a disclosed method of reducing the need for surgical intervention can further comprise measuring aortic root diameter, measuring aortic root cross-sectional area, measuring left ventricular volume, measuring lung architecture, measuring the degree of kyphosis, or any combination thereof.

In an aspect, a disclosed method of reducing the need for surgical intervention can comprise obtaining a biosample from the subject. In an aspect, a disclosed method of treating and/or preventing hereditary aortopathy can comprise measuring the level of one or more biomarkers in a biosample obtained from the subject. In an aspect, disclosed biomarkers can comprise circTGFB2R, miR-29a, KLF4, or any combination thereof.

In an aspect of a disclosed method of reducing the need for surgical intervention, if the level of circTGFB2R in the subject's biosample is similar to the reference level of circTGFB2R, then the risk of the subject developing an aortic aneurysm and/or experiencing an aortic dissection is minimized. In an aspect of a disclosed method of reducing the need for surgical intervention, if the level of circTGFB2R in the subject's biosample is less than the reference level of circTGFB2R, then the risk of the subject developing an aortic aneurysm and/or experiencing an aortic dissection is increased. In an aspect of a disclosed method of reducing the need for surgical intervention, if the level of MiR-29a in the subject's biosample is similar to the reference level of MiR-29a, then the risk of the subject developing an aortic aneurysm and/or experiencing an aortic dissection is minimized. In an aspect of a disclosed method of reducing the need for surgical intervention, if the level of MiR-29a in the subject's biosample is greater than the reference level of MiR-29a, then the risk of the subject developing an aortic aneurysm and/or experiencing an aortic dissection is increased. In an aspect of a disclosed method of reducing the need for surgical intervention, if the level of circTGFB2R in the subject's biosample is similar to the reference level of circTGFB2R and the level of MiR-29a is greater than the reference level of MiR-29, then the risk of the subject developing an aortic aneurysm and/or experiencing an aortic dissection is minimized. In an aspect of a disclosed method of reducing the need for surgical intervention, if the level of KLF4 in the subject's biosample is greater than the reference level of KLF4, then the risk of the subject developing an aortic aneurysm and/or experiencing an aortic dissection is increased. In an aspect, a disclosed method can further comprise increasing the level of and/or expression of circTGFB2R.

In an aspect, a disclosed phenotype switch vascular smooth muscle cells (VSMCs) can be prevented. In an aspect, a disclosed phenotype switch in VSMCs can comprise a switch from a contractile phenotype of to a synthetic phenotype. In an aspect, a disclosed contractile phenotype can comprise expression of one or more differentiation markers. In an aspect, disclosed differentiation markers can comprise SM22α and/or α-SMA. In an aspect, a disclosed synthetic phenotype can comprise expression of one or more synthetic markers. In an aspect, disclosed synthetic markers can comprise osteopontin (OPN) and/or matrix Gla protein (MGP).

In an aspect of a disclosed method of reducing the need for surgical intervention, administering a composition comprising a therapeutically effective amount of an angiotensin II receptor antagonist can inhibit the expression of one or more synthetic markers. In an aspect of a disclosed method of reducing the need for surgical intervention, administering a composition comprising a therapeutically effective amount of a TRPC4 antagonist can inhibit the expression of one or more synthetic markers. In an aspect of a method of reducing the need for surgical intervention, administering a composition comprising a therapeutically effective amount of an angiotensin II receptor antagonist and a therapeutically effective amount of a TRPC4 antagonist can inhibit the expression of one or more synthetic markers. In an aspect of a disclosed method of reducing the need for surgical intervention, administering a therapeutically effective amount of an angiotensin II receptor antagonist or a therapeutically effective amount of a TRPC4 antagonist can inhibit the expression of one or more synthetic markers.

In an aspect, a disclosed method of reducing the need for surgical intervention can comprise measuring the expression level of one or more markers of aortopathy in a biosample obtained from the subject. In an aspect, disclosed markers of aortopathy can comprise Smad2, Smad3, JNK1, ERK1/2, ERK5, MEK1, ROCK1, p38, MMP2, MMP9, MMP17, TRPC4, or any combination thereof. In an aspect, a disclosed expression level can comprise the level of protein expression, the level of miRNA expression, the level of mRNA expression, or the combination thereof. Methods and techniques for determining the expression and/or activity level of a disclosed biomarker, protein, peptide, microRNA (mi-R), messenger RNA (mRNA), DNA, or any combination thereof are known to the art and are disclosed herein.

In an aspect, a biosample can comprise a tissue or tissues, a cell or cells, a biopsied tissue or tissues, a biopsied cell or cells, blood, lymph, CFS, serum, plasma, urine, saliva, mucus, tears, or a combination thereof.

In an aspect, a disclosed method reducing the need for surgical intervention can further comprise generating one or more times a metabolic or genomic profile for the subject.

In an aspect, a disclosed method of reducing the need for surgical intervention can further comprise generating one or more times a proteomic profile for the subject. For example, a proteomic profile can be generated for all or for some of the biosamples obtained from the subject. In an aspect, a proteomic profile can be generated prior to a disclosed administering step, after a disclosed administering step, or both.

As used herein, proteomic profile can mean the combination of proteins found in a subject's biosample. The proteomic profile is a collection of measurements, such as but not limited to a quantity or concentration, for individual proteins taken from a subject's biosample. Techniques to determine the levels of individual components of the proteomic profile from biosamples are well known to the skilled technician and include, but are not limited to, mass spectrometry, ultra-performance liquid chromatography (UPLC), high-performance liquid chromatography (HPLC), mass spectrometry in conjunction with UPLC, LC/MS/MS, ELISA, and Western blots.

In an aspect, a disclosed method of reducing the need for surgical intervention can further comprise generating one or more times a miRNA profile for the subject. For example, a miRNA profile can be generated for all or for some of the biosamples obtained from the subject. In an aspect, a miRNA profile can be generated prior to a disclosed administering step, after a disclosed administering step, or both. As used herein, miRNA profile can mean the combination of miRNAs found in a subject's biosample. The miRNA profile is a collection of measurements, such as but not limited to a quantity or concentration, for individual miRNAs taken from a subject's biosample. Techniques to determine the levels of individual components of the miRNA profile from biosamples are well known to the skilled technician and include, but are not limited to, RNAseq and RT-qPCR.

The assessment of the levels of the individual components of the proteomic and/or miRNA profile can be expressed as absolute or relative values and may or may not be expressed in relation to another component, a standard, an internal standard, or another molecule of compound known to be in the sample. If the levels are assessed as relative to a standard or internal standard, then the standard can be added to the test sample prior to, during, or after sample processing.

The level of differential expression of proteins and/or miRNAs in a biosample when compared to a reference biosample (or any other biosample) can vary. For example, the level of any one or more differentially expressed proteins (such as, for example, circTGFB2R) and/or miRNAs (such as, for example miR-29a) in a biosample can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold lower than that of a reference biosample. Or the levels of any one or more differentially expressed proteins and/or miRNAs can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold higher than that of a reference biosample. In an aspect, the number of “times” the level of one or more differentially expressed proteins and/or miRNAs is lower or higher than that of a reference level can be a relative or an absolute number of times. Or, in an aspect, the level of the proteins and/or miRNAs can be normalized to a standard and these normalized levels can then be compared to one another to determine whether the differentially expressed proteins and/or miRNAs is lower or higher.

In an aspect, administering can comprise titrating a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects. In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a therapeutically effective dose of the disclosed angiotensin II receptor antagonist can comprise about 0.01 mg/kg/day to about 200 mg/kg/day. In an aspect, a therapeutically effective dose of the disclosed angiotensin II receptor antagonist can comprise about 50 mg/kg/day to about 150 mg/kg/day. In an aspect, a therapeutically effective dose of the disclosed angiotensin II receptor antagonist can comprise about 100 mg/kg/day to about 200 mg/kg/day. In an aspect, a therapeutically effective dose of the disclosed angiotensin II receptor antagonist can comprise about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day.

In an aspect, a therapeutically effective dose of a disclosed TRPC4 antagonist can comprise about 0.01 mg/kg/day to about 200 mg/kg/day. In an aspect, a therapeutically effective dose of a disclosed TRPC4 antagonist can comprise about 1 mg/kg/day to about 20 mg/kg/day. In an aspect, a therapeutically effective dose of a disclosed TRPC4 antagonist can comprise about 50 mg/kg/day to about 150 mg/kg/day. In an aspect, a therapeutically effective dose of a disclosed TRPC4 antagonist can comprise about 100 mg/kg/day to about 200 mg/kg/day. In an aspect, a therapeutically effective dose of a disclosed TRPC4 antagonist can comprise about 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day. In an aspect, a therapeutically effective dose of a disclosed TRPC4 antagonist can comprise about 10 mg/kg/day.

In an aspect, a disclosed method of reducing the need for surgical intervention can further comprise increasing and/or decreasing the expression level of one or more aspects of the signaling cascade presented in FIG. 9.

In an aspect, a disclosed method of reducing the need for surgical intervention can further comprise (i) treating and/or preventing an aortic aneurysm, (ii) treating and/or preventing hereditary aortopathy, (iii) slowing and/or inhibiting disease progression, (iv) identifying the risk of aortic aneurysm and/or aortic dissection, or (v) any combination thereof.

In an aspect, a disclosed method can comprise repeating an administering step one or more times. For example, a disclosed TRPC4 antagonist can be administered one or more times. In an aspect, a disclosed angiotensin II receptor antagonist can be administered one or more times. In an aspect, a disclosed composition or pharmaceutical composition comprising a disclosed TRPC4 antagonist and/or a disclosed angiotensin II receptor antagonist can be administered one or more times. In an aspect, administering of any disclosed antagonist, disclosed composition, and/or disclosed pharmaceutical formulation can comprise intravenous administration or oral administration.

In an aspect, the number of surgeries required by the subject in need thereof can be reduced by 1 surgery, 2 surgeries, 3 surgeries, 4 surgeries, 5 surgeries, or more than 5 surgeries than a subject not being subjected to or exposed to a disclosed method. For example, a subject in thereof can be subjected to 1 fewer surgery, 2 fewer surgeries, 3 fewer surgeries, 4 fewer surgeries, 5 fewer surgeries, or more than a subject not being subjected to or exposed to a disclosed method. In an aspect, the number of surgeries required by the subject in need thereof can be reduced by 10%, 20%, 30%, 40%, 50%, 60%, 70%, 80%, 90%, or more than 90% when compared to a subject not being subjected to or exposed to a disclosed method. In an aspect, the number of surgeries required by the subject in need thereof can be reduced by 10%-30%, 30%-60%, 60%-90%, or more than 90% when compared to a subject not being subjected to or exposed to a disclosed method. In an aspect, a disclosed subject can be subjected to a disclosed method and not require additional surgeries.

In an aspect of a disclosed method, the number of surgeries required by the subject in need thereof can be reduced when compared to the number of surgeries required by a subject not receiving a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a disclosed method of reducing the need for surgical intervention can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both. In an aspect, modifying the treating step can comprise changing the amount of a disclosed angiotensin II receptor antagonist, the amount of a disclosed TRPC4 antagonist, or the amount of a disclosed composition comprising an angiotensin II receptor antagonist and a TRPC4 antagonist administered to the subject, changing the frequency of administration of a disclosed angiotensin II receptor antagonist, the amount of a disclosed TRPC4 antagonist, or the amount of a disclosed composition comprising an angiotensin II receptor antagonist and a TRPC4 antagonist, changing the route of administration of a disclosed angiotensin II receptor antagonist, the amount of a disclosed TRPC4 antagonist, or the amount of a disclosed composition comprising an angiotensin II receptor antagonist and a TRPC4 antagonist, or any combination thereof. In an aspect, modifying the administering step can comprise changing the amount of a disclosed angiotensin II receptor and/or a disclosed TRPC4 antagonist, a disclosed composition comprising a disclosed angiotensin II receptor and/or a disclosed TRPC4 antagonist.

In an aspect, a disclosed method of reducing the need for surgical intervention can comprise improving and/or increasing the life expectancy of a disclosed subject or a disclosed patient. In an aspect of a disclosed method, the life expectancy of the recipient or subject in need thereof is increased. In an aspect, life expectancy can be increased by 5-10 years, by 10-15 years, by 15-20 years, by 20-25 years, by 25-30 years, by 30-35 years, by 35-40 years, or more than 40 years when compared to a subject not being subjected to or exposed to a disclosed method.

In an aspect, a disclosed method of reducing the need for surgical intervention can further comprise improving the subject's hemodynamics. Hemodynamics are known to the skill in the art. In an aspect, hemodynamics can comprise blow flow in the aortic root, sinuses, coronary orifices, or any combination thereof.

In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects.

In an aspect, a disclosed method of treating and/or preventing hereditary aortopathy can further comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof in a specific or disclosed subject. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects for a specific or disclosed subject.

In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, the growth of an aneurysm can be measured using echocardiography, imaging techniques (e.g., MRI and/or computed tomography), abdominal or pelvic ultrasonography, or any combination thereof. In an aspect, aortic aneurysm size can be a critical determinant of the need for intervention, yet the maximal diameter can often vary depending on the modality and method of measurement. In an aspect, a CT scan of a disclosed subject or disclosed patient can comprise using a three-dimensional reconstruction software. In an aspect, maximal aortic diameter can be recorded in the anteroposterior (CT-AP) plane, the maximal ellipse (CT-ME), perpendicular to the maximal ellipse (CT-PME), or perpendicular to the centerline of flow (CT-PCLF), or any combination thereof. In an aspect, a disclosed aneurysm can be analyzed according to the principles of Bland and Altman.

In an aspect, a disclosed method of reducing the need for surgical intervention can further comprise administering to the subject (i) one or more active agents, (ii) biologically active agents, (iii) one or more pharmaceutically active agents, (iv) one or more immune-based therapeutic agents, (v) one or more clinically approved agents, or (vi) a combination thereof.

### I. Methods of Slowing and/or Inhibiting Disease Progressions

Disclosed herein is a method of slowing and/or inhibiting disease progression, the method comprising administering to a subject having a hereditary aortopathy a therapeutically effective amount of an angiotensin II receptor antagonist; and administering to the subject a therapeutically effective amount of a TRPC4 antagonist, wherein the subject has an aortic aneurysm that does not grow and/or wherein the subject does not develop an aortic dissection.

Disclosed herein is a method of slowing and/or inhibiting disease progression, the method comprising administering to a subject in need thereof a therapeutically effective amount of an angiotensin II receptor antagonist; and administering to the subject a therapeutically effective amount of a TRPC4 antagonist, wherein the subject has an aortic aneurysm that does not grow and/or wherein the subject does not develop an aortic dissection.

Disclosed herein is a method of slowing and/or inhibiting disease progression, the method comprising administering to a subject having a hereditary aortopathy a composition comprising a therapeutically effective amount of an angiotensin II receptor antagonist and a therapeutically effective amount of a TRPC4 antagonist, wherein the subject has an aortic aneurysm that does not grow and/or wherein the subject does not develop an aortic dissection.

Disclosed herein is a method of slowing and/or inhibiting disease progression, the method comprising administering to a subject in need thereof a composition comprising a therapeutically effective amount of an angiotensin II receptor antagonist and a therapeutically effective amount of a TRPC4 antagonist, wherein the subject has an aortic aneurysm that does not grow and/or wherein the subject does not develop an aortic dissection.

In an aspect, a disclosed composition comprising a therapeutically effective amount of an angiotensin II receptor antagonist and a therapeutically effective amount of a TRPC4 antagonist can comprise one or more pharmaceutically acceptable carriers and/or excipients.

Pharmaceutically acceptable carriers and/or excipients are known to the skilled person in the art and are discussed supra.

In an aspect, a disclosed angiotensin II receptor antagonist can be administered prior to the TRPC4 antagonist, concurrently with the TRPC4 antagonist, or after the TRPC4 antagonist. In an aspect, the timing of administration of a disclosed TRPC4 antagonist can be modified.

In an aspect, a disclosed angiotensin II receptor antagonist can comprise azilsartan or azilsartan medoxomil, candesartan, eprosartan, irbesartan, losartan, olmesartan, saprisartan, telmisartan, valsartan, any pharmaceutical salt thereof, or any combination thereof. In an aspect, a disclosed angiotensin II receptor can comprise losartan. Angiotensin II receptor antagonists are known to the art.

In an aspect a disclosed TRPC4 antagonist can comprise ML204, HC-070, or the combination thereof. In an aspect, a disclosed TRPC4 antagonist can comprise any antagonist identified using OSPREY or identified pursuant to Example 3. TRPC4 is known to the art. For example, in an aspect, TRPC4 can comprise the nucleotide sequence set forth in SEQ ID NO:06 or a fragment thereof. In an aspect, the cDNA for TRPC4 can comprise the sequence set forth in SEQ ID NO:07 or a fragment thereof. In an aspect, a disclosed encoded TRPC4 can comprise the sequence set forth in SEQ ID NO:04 or SEQ ID NO:05 or a fragment thereof. Similarly, in an aspect, Trpc4 can comprise the nucleotide sequence set forth in SEQ ID NO:12 or a fragment thereof. In an aspect, the cDNA for Trpc4 can comprise the sequence set forth in SEQ ID NO:13 or a fragment thereof. In an aspect, a disclosed encoded TRPC4 can comprise the sequence set forth in SEQ ID NO:11 or a fragment thereof.

In an aspect a disclosed TRPC4 antagonist can comprise a ML204. In an aspect, ML204 is 4-Methyl-2-(piperidin-1-yl) quinoline (PubChem CID 230710) and comprises the molecular formula C₁₅H₁₈N₂. The structure of ML204 is provided supra. In an aspect a disclosed TRPC4 antagonist can comprise a HC-070. In an aspect, HC-070 is 8-(3-chlorophenoxy)-7-[(4-chlorophenyl)methyl]-1-(3-hydroxypropyl)-3-methylpurine-2,6-dione (PubChem CID 85473309) and comprises the molecular formula C₂₂H₂₀Cl₂N₄O₄. The structure of HC-070 is provided supra.

In an aspect, a disclosed method can comprise validating and/or characterizing the pharmacokinetic profile of a disclosed TRPC4 antagonist identified using OSPREY. In an aspect, validating and/or characterizing a disclosed TRPC4 antagonist can comprise using an animal model such as the disclosed aMFS mouse model.

In an aspect, a disclosed method of slowing and/or inhibiting disease progression can further comprise surgically repairing the aortic aneurysm and/or repairing the aortic dissection. In an aspect, surgical intervention can comprise endovascular aortic aneurysm repair (EVAR). In an aspect, if the diameter of the subject's aorta is about 50 millimeters, then surgical intervention is required. In an aspect, surgically repairing the aortic aneurysm and/or repairing the aortic dissection can comprise replacing the subject's aortic valve, the subject's aortic root, or the combination thereof.

In an aspect, a disclosed subject can have a dominant negative mutation in the FBN1 gene. In an aspect, a disclosed subject can have a haploinsufficient mutation in the FBN1 gene. FBN1 is known to the art. For example, in an aspect, FBN1 can comprise the nucleotide sequence set forth in SEQ ID NO:02 or a fragment thereof. In an aspect, the cDNA for FBN1 can comprise the sequence set forth in SEQ ID NO:03 or a fragment thereof. In an aspect, a disclosed encoded FBN1 can comprise the sequence set forth in SEQ ID NO:01 or a fragment thereof. Similarly, in an aspect, FBN1 can comprise the nucleotide sequence set forth in SEQ ID NO:09 or a fragment thereof. In an aspect, the cDNA for FBN1 can comprise the sequence set forth in SEQ ID NO:10 or a fragment thereof. In an aspect, a disclosed encoded FBN1 can comprise the sequence set forth in SEQ ID NO:08 or a fragment thereof.

In an aspect, a disclosed subject can have one or more genomic aberrations in the fibrillin-1 (FBN1) gene. Genomic aberrations can comprise mutations, rearrangements, insertions, deletion, amplifications, or any combination thereof. In an aspect of a disclosed method, a disclosed subject in need thereof can be a human being. In an aspect, a human being can be a neonate, a child, an adolescent, or an adult. In an aspect, a disclosed subject can be diagnosed with or at risk of having Marfan syndrome. In an aspect, a disclosed subject is seeking medical intervention and/or treatment.

In an aspect, a method of slowing and/or inhibiting disease progression can further comprise measuring aortic root diameter, measuring aortic root cross-sectional area, measuring left ventricular volume, measuring lung architecture, measuring the degree of kyphosis, or any combination thereof.

In an aspect, a disclosed method of slowing and/or inhibiting disease progression can comprise obtaining a biosample from the subject. In an aspect, a disclosed method of slowing and/or inhibiting disease progression can comprise measuring the level of one or more biomarkers in a biosample obtained from the subject. In an aspect, disclosed biomarkers can comprise circTGFB2R, miR-29a, KLF4, or any combination thereof.

In an aspect of a disclosed method of slowing and/or inhibiting disease progression if the level of circTGFB2R in the subject's biosample is similar to the reference level of circTGFB2R, then the risk of the subject developing an aortic aneurysm and/or experiencing an aortic dissection is minimized. In an aspect of a disclosed method of slowing and/or inhibiting disease progression, if the level of circTGFB2R in the subject's biosample is less than the reference level of circTGFB2R, then the risk of the subject developing an aortic aneurysm and/or experiencing an aortic dissection is increased. In an aspect of a disclosed method of slowing and/or inhibiting disease progression, if the level of MiR-29a in the subject's biosample is similar to the reference level of MiR-29a, then the risk of the subject developing an aortic aneurysm and/or experiencing an aortic dissection is minimized. In an aspect of a disclosed method of slowing and/or inhibiting disease progression, if the level of MiR-29a in the subject's biosample is greater than the reference level of MiR-29a, then the risk of the subject developing an aortic aneurysm and/or experiencing an aortic dissection is increased. In an aspect of a disclosed method of slowing and/or inhibiting disease progression, if the level of circTGFB2R in the subject's biosample is similar to the reference level of circTGFB2R and the level of MiR-29a is greater than the reference level of MiR-29, then the risk of the subject developing an aortic aneurysm and/or experiencing an aortic dissection is minimized. In an aspect of a disclosed method of slowing and/or inhibiting disease progression, if the level of KLF4 in the subject's biosample is greater than the reference level of KLF4, then the risk of the subject developing an aortic aneurysm and/or experiencing an aortic dissection is increased. In an aspect, a disclosed method can further comprise increasing the level of and/or expression of circTGFB2R.

In an aspect, a disclosed phenotype switch vascular smooth muscle cells (VSMCs) can be prevented. In an aspect, a disclosed phenotype switch in VSMCs can comprise a switch from a contractile phenotype of to a synthetic phenotype. In an aspect, a disclosed contractile phenotype can comprise expression of one or more differentiation markers. In an aspect, disclosed differentiation markers can comprise SM22α and/or α-SMA. In an aspect, a disclosed synthetic phenotype can comprise expression of one or more synthetic markers. In an aspect, disclosed synthetic markers can comprise osteopontin (OPN) and/or matrix Gla protein (MGP).

In an aspect of a disclosed method of slowing and/or inhibiting disease progression, administering a composition comprising a therapeutically effective amount of an angiotensin II receptor antagonist can inhibit the expression of one or more synthetic markers. In an aspect of a disclosed method of slowing and/or inhibiting disease progression, administering a composition comprising a therapeutically effective amount of a TRPC4 antagonist can inhibit the expression of one or more synthetic markers. In an aspect of a disclosed method of slowing and/or inhibiting disease progression, administering a composition comprising a therapeutically effective amount of an angiotensin II receptor antagonist and a therapeutically effective amount of a TRPC4 antagonist can inhibit the expression of one or more synthetic markers. In an aspect of a disclosed method of slowing and/or inhibiting disease progression, administering a therapeutically effective amount of an angiotensin II receptor antagonist or a therapeutically effective amount of a TRPC4 antagonist can inhibit the expression of one or more synthetic markers.

In an aspect, a disclosed method of slowing and/or inhibiting disease progression can comprise measuring the expression level of one or more markers of aortopathy in a biosample obtained from the subject. In an aspect, disclosed markers of aortopathy can comprise Smad2, Smad3, JNK1, ERK1/2, ERK5, MEK1, ROCK1, p38, MMP2, MMP9, MMP17, TRPC4, or any combination thereof. In an aspect, a disclosed expression level can comprise the level of protein expression, the level of miRNA expression, the level of mRNA expression, or the combination thereof. Methods and techniques for determining the expression and/or activity level of a disclosed biomarker, protein, peptide, microRNA (mi-R or miRNA), messenger RNA (mRNA), DNA, or any combination thereof are known to the art and are disclosed herein.

In an aspect, a biosample can comprise a tissue or tissues, a cell or cells, a biopsied tissue or tissues, a biopsied cell or cells, blood, lymph, CFS, serum, plasma, urine, saliva, mucus, tears, or a combination thereof.

In an aspect, a disclosed method of slowing and/or inhibiting disease progression can further comprise generating one or more times a metabolic or genomic profile for the subject.

In an aspect, a disclosed method of slowing and/or inhibiting disease progression can further comprise generating one or more times a proteomic profile for the subject. For example, a proteomic profile can be generated for all or for some of the biosamples obtained from the subject. In an aspect, a proteomic profile can be generated prior to a disclosed administering step, after a disclosed administering step, or both.

As used herein, proteomic profile can mean the combination of proteins found in a subject's biosample. The proteomic profile is a collection of measurements, such as but not limited to a quantity or concentration, for individual proteins taken from a subject's biosample.

Techniques to determine the levels of individual components of the proteomic profile from biosamples are well known to the skilled technician and include, but are not limited to, mass spectrometry, ultra-performance liquid chromatography (UPLC), high-performance liquid chromatography (HPLC), mass spectrometry in conjunction with UPLC, LC/MS/MS, ELISA, and Western blots.

In an aspect, a disclosed method of slowing and/or inhibiting disease progression can further comprise generating one or more times a miRNA profile for the subject. For example, a miRNA profile can be generated for all or for some of the biosamples obtained from the subject. In an aspect, a miRNA profile can be generated prior to a disclosed administering step, after a disclosed administering step, or both. As used herein, miRNA profile can mean the combination of miRNAs found in a subject's biosample. The miRNA profile is a collection of measurements, such as but not limited to a quantity or concentration, for individual miRNAs taken from a subject's biosample. Techniques to determine the levels of individual components of the miRNA profile from biosamples are well known to the skilled technician and include, but are not limited to, RNAseq and RT-qPCR.

The assessment of the levels of the individual components of the proteomic and/or miRNA profile can be expressed as absolute or relative values and may or may not be expressed in relation to another component, a standard, an internal standard, or another molecule of compound known to be in the sample. If the levels are assessed as relative to a standard or internal standard, then the standard can be added to the test sample prior to, during, or after sample processing.

The level of differential expression of proteins and/or miRNAs in a biosample when compared to a reference biosample (or any other biosample) can vary. For example, the level of any one or more differentially expressed proteins (such as, for example, circTGFB2R) and/or miRNAs (such as, for example miR-29a) in a biosample can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold lower than that of a reference biosample. Or the levels of any one or more differentially expressed proteins and/or miRNAs can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold higher than that of a reference biosample. In an aspect, the number of “times” the level of one or more differentially expressed proteins and/or miRNAs is lower or higher than that of a reference level can be a relative or an absolute number of times. Or, in an aspect, the level of the proteins and/or miRNAs can be normalized to a standard and these normalized levels can then be compared to one another to determine whether the differentially expressed proteins and/or miRNAs is lower or higher.

In an aspect, administering can comprise titrating a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects. In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a therapeutically effective dose of the disclosed angiotensin II receptor antagonist can comprise about 0.01 mg/kg/day to about 200 mg/kg/day. In an aspect, a therapeutically effective dose of the disclosed angiotensin II receptor antagonist can comprise about 50 mg/kg/day to about 150 mg/kg/day. In an aspect, a therapeutically effective dose of the disclosed angiotensin II receptor antagonist can comprise about 100 mg/kg/day to about 200 mg/kg/day. In an aspect, a therapeutically effective dose of the disclosed angiotensin II receptor antagonist can comprise about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day.

In an aspect, a therapeutically effective dose of a disclosed TRPC4 antagonist can comprise about 0.01 mg/kg/day to about 200 mg/kg/day. In an aspect, a therapeutically effective dose of a disclosed TRPC4 antagonist can comprise about 1 mg/kg/day to about 20 mg/kg/day. In an aspect, a therapeutically effective dose of a disclosed TRPC4 antagonist can comprise about 50 mg/kg/day to about 150 mg/kg/day. In an aspect, a therapeutically effective dose of a disclosed TRPC4 antagonist can comprise about 100 mg/kg/day to about 200 mg/kg/day. In an aspect, a therapeutically effective dose of a disclosed TRPC4 antagonist can comprise about 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day. In an aspect, a therapeutically effective dose of a disclosed TRPC4 antagonist can comprise about 10 mg/kg/day.

In an aspect, a disclosed method of slowing and/or inhibiting disease progression can further comprise increasing and/or decreasing the expression level of one or more aspects of the signaling cascade presented in FIG. 9.

In an aspect, a disclosed method of slowing and/or inhibiting disease progression can further comprise (i) treating and/or preventing an aortic aneurysm, (ii) reducing the need for surgical intervention, (iii) treating and/or preventing hereditary aortopathy, (iv) identifying the risk of aortic aneurysm and/or aortic dissection, or (v) any combination thereof.

In an aspect, a disclosed method can comprise repeating an administering step one or more times. For example, a disclosed TRPC4 antagonist can be administered one or more times. In an aspect, a disclosed angiotensin II receptor antagonist can be administered one or more times. In an aspect, a disclosed composition or pharmaceutical composition comprising a disclosed TRPC4 antagonist and/or a disclosed angiotensin II receptor antagonist can be administered one or more times. In an aspect, administering of any disclosed antagonist, disclosed composition, and/or disclosed pharmaceutical formulation can comprise intravenous administration or oral administration.

In an aspect, the number of surgeries required by the subject in need thereof can be reduced by 1 surgery, 2 surgeries, 3 surgeries, 4 surgeries, 5 surgeries, or more than 5 surgeries than a subject not being subjected to or exposed to a disclosed method. For example, a subject in thereof can be subjected to 1 fewer surgery, 2 fewer surgeries, 3 fewer surgeries, 4 fewer surgeries, 5 fewer surgeries, or more than a subject not being subjected to or exposed to a disclosed method. In an aspect, the number of surgeries required by the subject in need thereof can be reduced by 10%, 20%, 30%, 40%, 50%, 60%, 70%, 80%, 90%, or more than 90% when compared to a subject not being subjected to or exposed to a disclosed method. In an aspect, the number of surgeries required by the subject in need thereof can be reduced by 10%-30%, 30%-60%, 60%-90%, or more than 90% when compared to a subject not being subjected to or exposed to a disclosed method. In an aspect, a disclosed subject can be subjected to a disclosed method and not require additional surgeries.

In an aspect of a disclosed method, the number of surgeries required by the subject in need thereof can be reduced when compared to the number of surgeries required by a subject not receiving a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a disclosed method of slowing and/or inhibiting disease progression can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both. In an aspect, modifying the treating step can comprise changing the amount of a disclosed angiotensin II receptor antagonist, the amount of a disclosed TRPC4 antagonist, or the amount of a disclosed composition comprising an angiotensin II receptor antagonist and a TRPC4 antagonist administered to the subject, changing the frequency of administration of a disclosed angiotensin II receptor antagonist, the amount of a disclosed TRPC4 antagonist, or the amount of a disclosed composition comprising an angiotensin II receptor antagonist and a TRPC4 antagonist, changing the route of administration of a disclosed angiotensin II receptor antagonist, the amount of a disclosed TRPC4 antagonist, or the amount of a disclosed composition comprising an angiotensin II receptor antagonist and a TRPC4 antagonist, or any combination thereof. In an aspect, modifying the administering step can comprise changing the amount of a disclosed angiotensin II receptor and/or a disclosed TRPC4 antagonist, a disclosed composition comprising a disclosed angiotensin II receptor and/or a disclosed TRPC4 antagonist.

In an aspect, a disclosed method of slowing and/or inhibiting disease progression can comprise improving and/or increasing the life expectancy of a disclosed subject or a disclosed patient. In an aspect of a disclosed method, the life expectancy of the recipient or subject in need thereof is increased. In an aspect, life expectancy can be increased by 5-10 years, by 10-15 years, by 15-20 years, by 20-25 years, by 25-30 years, by 30-35 years, by 35-40 years, or more than 40 years when compared to a subject not being subjected to or exposed to a disclosed method.

In an aspect, a method of slowing and/or inhibiting disease progression can further comprise improving the subject's hemodynamics. Hemodynamics are known to the skill in the art. In an aspect, hemodynamics can comprise blow flow in the aortic root, sinuses, coronary orifices, or any combination thereof.

In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects.

In an aspect, a disclosed method of slowing and/or inhibiting disease progression can further comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof in a specific or disclosed subject. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects for a specific or disclosed subject.

In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, the growth of an aneurysm can be measured using echocardiography, imaging techniques (e.g., MRI and/or computed tomography), abdominal or pelvic ultrasonography, or any combination thereof. In an aspect, aortic aneurysm size can be a critical determinant of the need for intervention, yet the maximal diameter can often vary depending on the modality and method of measurement. In an aspect, a CT scan of a disclosed subject or disclosed patient can comprise using a three-dimensional reconstruction software. In an aspect, maximal aortic diameter can be recorded in the anteroposterior (CT-AP) plane, the maximal ellipse (CT-ME), perpendicular to the maximal ellipse (CT-PME), or perpendicular to the centerline of flow (CT-PCLF), or any combination thereof. In an aspect, a disclosed aneurysm can be analyzed according to the principles of Bland and Altman.

In an aspect, a disclosed method of slowing and/or inhibiting disease progression can further comprise administering to the subject (i) one or more active agents, (ii) biologically active agents, (iii) one or more pharmaceutically active agents, (iv) one or more immune-based therapeutic agents, (v) one or more clinically approved agents, or (vi) a combination thereof.

### J. Methods of Identifying a Subject at Risk of Aortic Aneurysm and/or Aortic Dissection

Disclosed herein is a method of identifying a subject at risk of aortic aneurysm and/or aortic dissection, the method comprising measuring the level of one or more biomarkers in a biosample obtained from a subject; and if the level of the one or more biomarkers is modulated when compared to a control level, then administering to a subject having a hereditary aortopathy a therapeutically effective amount of an angiotensin II receptor antagonist; and administering to the subject a therapeutically effective amount of a TRPC4 antagonist, wherein risk of the subject developing an aortic aneurysm and/or experiencing an aortic dissection is minimized.

Disclosed herein is a method of identifying a subject at risk of aortic aneurysm and/or aortic dissection, the method comprising measuring the level of one or more biomarkers in a biosample obtained from a subject; and if the level of the one or more biomarkers is modulated when compared to a control level, then administering to a subject having a hereditary aortopathy a composition comprising a therapeutically effective amount of an angiotensin II receptor antagonist and a therapeutically effective amount of a TRPC4 antagonist, wherein risk of the subject developing an aortic aneurysm and/or experiencing an aortic dissection is minimized.

In an aspect, modulate or modulated can comprise an increase in the level of one or more biomarkers when compared to a control level. In an aspect, modulate or modulated can comprise an decrease in the level of one or more biomarkers when compared to a control level.

In an aspect, a disclosed composition comprising a therapeutically effective amount of an angiotensin II receptor antagonist and a therapeutically effective amount of a TRPC4 antagonist can comprise one or more pharmaceutically acceptable carriers and/or excipients.

Pharmaceutically acceptable carriers and/or excipients are known to the skilled person in the art and are discussed supra.

In an aspect, a disclosed angiotensin II receptor antagonist can be administered prior to the TRPC4 antagonist, concurrently with the TRPC4 antagonist, or after the TRPC4 antagonist. In an aspect, the timing of administration of a disclosed TRPC4 antagonist can be modified.

In an aspect, a disclosed angiotensin II receptor antagonist can comprise azilsartan or azilsartan medoxomil, candesartan, eprosartan, irbesartan, losartan, olmesartan, saprisartan, telmisartan, valsartan, any pharmaceutical salt thereof, or any combination thereof. In an aspect, a disclosed angiotensin II receptor can comprise losartan. Angiotensin II receptor antagonists are known to the art.

In an aspect a disclosed TRPC4 antagonist can comprise ML204, HC-070, or the combination thereof. In an aspect, a disclosed TRPC4 antagonist can comprise any antagonist identified using OSPREY or identified pursuant to Example 3. TRPC4 is known to the art. For example, in an aspect, TRPC4 can comprise the nucleotide sequence set forth in SEQ ID NO:06 or a fragment thereof. In an aspect, the cDNA for TRPC4 can comprise the sequence set forth in SEQ ID NO:07 or a fragment thereof. In an aspect, a disclosed encoded TRPC4 can comprise the sequence set forth in SEQ ID NO:04 or SEQ ID NO:05 or a fragment thereof. Similarly, in an aspect, Trpc4 can comprise the nucleotide sequence set forth in SEQ ID NO:12 or a fragment thereof. In an aspect, the cDNA for Trpc4 can comprise the sequence set forth in SEQ ID NO:13 or a fragment thereof. In an aspect, a disclosed encoded TRPC4 can comprise the sequence set forth in SEQ ID NO:11 or a fragment thereof.

In an aspect a disclosed TRPC4 antagonist can comprise a ML204. In an aspect, ML204 is 4-Methyl-2-(piperidin-1-yl) quinoline (PubChem CID 230710) and comprises the molecular formula C₁₅H₁₈N₂. The structure of ML204 is provided supra.

In an aspect a disclosed TRPC4 antagonist can comprise a HC-070. In an aspect, HC-070 is 8-(3-chlorophenoxy)-7-[(4-chlorophenyl)methyl]-1-(3-hydroxypropyl)-3-methylpurine-2,6-dione (PubChem CID 85473309) and comprises the molecular formula C₂₂H₂₀Cl₂N₄O₄. The structure of HC-070 is provided supra.

In an aspect, a disclosed method can comprise validating and/or characterizing the pharmacokinetic profile of a disclosed TRPC4 antagonist identified using OSPREY. In an aspect, validating and/or characterizing a disclosed TRPC4 antagonist can comprise using an animal model such as the disclosed aMFS mouse model.

In an aspect, a method of identifying a subject at risk of aortic aneurysm and/or aortic dissection can further comprise surgically repairing the aortic aneurysm and/or repairing the aortic dissection. In an aspect, surgical intervention can comprise endovascular aortic aneurysm repair (EVAR). In an aspect, if the diameter of the subject's aorta is about 50 millimeters, then surgical intervention is required. In an aspect, surgically repairing the aortic aneurysm and/or repairing the aortic dissection can comprise replacing the subject's aortic valve, the subject's aortic root, or the combination thereof.

In an aspect, a disclosed subject can have a dominant negative mutation in the FBN1 gene. In an aspect, a disclosed subject can have a haploinsufficient mutation in the FBN1 gene. FBN1 is known to the art. For example, in an aspect, FBN1 can comprise the nucleotide sequence set forth in SEQ ID NO:02 or a fragment thereof. In an aspect, the cDNA for FBN1 can comprise the sequence set forth in SEQ ID NO:03 or a fragment thereof. In an aspect, a disclosed encoded FBN1 can comprise the sequence set forth in SEQ ID NO:01 or a fragment thereof. Similarly, in an aspect, FBN1 can comprise the nucleotide sequence set forth in SEQ ID NO:09 or a fragment thereof. In an aspect, the cDNA for FBN1 can comprise the sequence set forth in SEQ ID NO:10 or a fragment thereof. In an aspect, a disclosed encoded FBN1 can comprise the sequence set forth in SEQ ID NO:08 or a fragment thereof.

In an aspect, a disclosed subject can have one or more genomic aberrations in the fibrillin-1 (FBN1) gene. Genomic aberrations can comprise mutations, rearrangements, insertions, deletion, amplifications, or any combination thereof. In an aspect of a disclosed method, a disclosed subject in need thereof can be a human being. In an aspect, a human being can be a neonate, a child, an adolescent, or an adult. In an aspect, a disclosed subject can be diagnosed with or at risk of having Marfan syndrome. In an aspect, a disclosed subject is seeking medical intervention and/or treatment.

In an aspect, a disclosed method of identifying a subject at risk of aortic aneurysm and/or aortic dissection can further comprise measuring aortic root diameter, measuring aortic root cross-sectional area, measuring left ventricular volume, measuring lung architecture, measuring the degree of kyphosis, or any combination thereof.

In an aspect, a disclosed method of identifying a subject at risk of aortic aneurysm and/or aortic dissection can comprise obtaining a biosample from the subject. In an aspect, a disclosed method of treating and/or preventing hereditary aortopathy can comprise measuring the level of one or more biomarkers in a biosample obtained from the subject. In an aspect, disclosed biomarkers can comprise circTGFB2R, miR-29a, KLF4, or any combination thereof.

In an aspect of a disclosed method of identifying a subject at risk of aortic aneurysm and/or aortic dissection, if the level of circTGFB2R in the subject's biosample is similar to the reference level of circTGFB2R, then the risk of the subject developing an aortic aneurysm and/or experiencing an aortic dissection is minimized. In an aspect of a disclosed method of identifying a subject at risk of aortic aneurysm and/or aortic dissection, if the level of circTGFB2R in the subject's biosample is less than the reference level of circTGFB2R, then the risk of the subject developing an aortic aneurysm and/or experiencing an aortic dissection is increased. In an aspect of a disclosed method of identifying a subject at risk of aortic aneurysm and/or aortic dissection, if the level of MiR-29a in the subject's biosample is similar to the reference level of MiR-29a, then the risk of the subject developing an aortic aneurysm and/or experiencing an aortic dissection is minimized. In an aspect of a disclosed method of identifying a subject at risk of aortic aneurysm and/or aortic dissection if the level of MiR-29a in the subject's biosample is greater than the reference level of MiR-29a, then the risk of the subject developing an aortic aneurysm and/or experiencing an aortic dissection is increased. In an aspect of a method of identifying a subject at risk of aortic aneurysm and/or aortic dissection, if the level of circTGFB2R in the subject's biosample is similar to the reference level of circTGFB2R and the level of MiR-29a is greater than the reference level of MiR-29, then the risk of the subject developing an aortic aneurysm and/or experiencing an aortic dissection is minimized. In an aspect of a disclosed method of identifying a subject at risk of aortic aneurysm and/or aortic dissection, if the level of KLF4 in the subject's biosample is greater than the reference level of KLF4, then the risk of the subject developing an aortic aneurysm and/or experiencing an aortic dissection is increased. In an aspect, a disclosed method can further comprise increasing the level of and/or expression of circTGFB2R.

In an aspect, a disclosed phenotype switch vascular smooth muscle cells (VSMCs) can be prevented. In an aspect, a disclosed phenotype switch in VSMCs can comprise a switch from a contractile phenotype of to a synthetic phenotype. In an aspect, a disclosed contractile phenotype can comprise expression of one or more differentiation markers. In an aspect, disclosed differentiation markers can comprise SM22α and/or α-SMA. In an aspect, a disclosed synthetic phenotype can comprise expression of one or more synthetic markers. In an aspect, disclosed synthetic markers can comprise osteopontin (OPN) and/or matrix Gla protein (MGP).

In an aspect of a disclosed method of identifying a subject at risk of aortic aneurysm and/or aortic dissection, administering a composition comprising a therapeutically effective amount of an angiotensin II receptor antagonist can inhibit the expression of one or more synthetic markers. In an aspect of a disclosed method of identifying a subject at risk of aortic aneurysm and/or aortic dissection, administering a composition comprising a therapeutically effective amount of a TRPC4 antagonist can inhibit the expression of one or more synthetic markers. In an aspect of a disclosed method of identifying a subject at risk of aortic aneurysm and/or aortic dissection administering a composition comprising a therapeutically effective amount of an angiotensin II receptor antagonist and a therapeutically effective amount of a TRPC4 antagonist can inhibit the expression of one or more synthetic markers. In an aspect of a disclosed method of identifying a subject at risk of aortic aneurysm and/or aortic dissection, administering a therapeutically effective amount of an angiotensin II receptor antagonist or a therapeutically effective amount of a TRPC4 antagonist can inhibit the expression of one or more synthetic markers.

In an aspect, a disclosed method of identifying a subject at risk of aortic aneurysm and/or aortic dissection can comprise measuring the expression level of one or more markers of aortopathy in a biosample obtained from the subject. In an aspect, disclosed markers of aortopathy can comprise Smad2, Smad3, JNK1, ERK1/2, ERK5, MEK1, ROCK1, p38, MMP2, MMP9, MMP17, TRPC4, or any combination thereof. In an aspect, a disclosed expression level can comprise the level of protein expression, the level of miRNA expression, the level of mRNA expression, or the combination thereof. Methods and techniques for determining the expression and/or activity level of a disclosed biomarker, protein, peptide, microRNA (mi-R), messenger RNA (mRNA), DNA, or any combination thereof are known to the art and are disclosed herein.

In an aspect, a biosample can comprise a tissue or tissues, a cell or cells, a biopsied tissue or tissues, a biopsied cell or cells, blood, lymph, CFS, serum, plasma, urine, saliva, mucus, tears, or a combination thereof.

In an aspect, a method of identifying a subject at risk of aortic aneurysm and/or aortic dissection can further comprise generating one or more times a metabolic or genomic profile for the subject.

In an aspect, a disclosed method of identifying a subject at risk of aortic aneurysm and/or aortic dissection can further comprise generating one or more times a proteomic profile for the subject. For example, a proteomic profile can be generated for all or for some of the biosamples obtained from the subject. In an aspect, a proteomic profile can be generated prior to a disclosed administering step, after a disclosed administering step, or both.

As used herein, proteomic profile can mean the combination of proteins found in a subject's biosample. The proteomic profile is a collection of measurements, such as but not limited to a quantity or concentration, for individual proteins taken from a subject's biosample. Techniques to determine the levels of individual components of the proteomic profile from biosamples are well known to the skilled technician and include, but are not limited to, mass spectrometry, ultra-performance liquid chromatography (UPLC), high-performance liquid chromatography (HPLC), mass spectrometry in conjunction with UPLC, LC/MS/MS, ELISA, and Western blots.

In an aspect, a disclosed method of identifying a subject at risk of aortic aneurysm and/or aortic dissection can further comprise generating one or more times a miRNA profile for the subject. For example, a miRNA profile can be generated for all or for some of the biosamples obtained from the subject. In an aspect, a miRNA profile can be generated prior to a disclosed administering step, after a disclosed administering step, or both. As used herein, miRNA profile can mean the combination of miRNAs found in a subject's biosample. The miRNA profile is a collection of measurements, such as but not limited to a quantity or concentration, for individual miRNAs taken from a subject's biosample. Techniques to determine the levels of individual components of the miRNA profile from biosamples are well known to the skilled technician and include, but are not limited to, RNAseq and RT-qPCR.

The assessment of the levels of the individual components of the proteomic and/or miRNA profile can be expressed as absolute or relative values and may or may not be expressed in relation to another component, a standard, an internal standard, or another molecule of compound known to be in the sample. If the levels are assessed as relative to a standard or internal standard, then the standard can be added to the test sample prior to, during, or after sample processing.

The level of differential expression of proteins and/or miRNAs in a biosample when compared to a reference biosample (or any other biosample) can vary. For example, the level of any one or more differentially expressed proteins (such as, for example, circTGFB2R) and/or miRNAs (such as, for example miR-29a) in a biosample can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold lower than that of a reference biosample. Or the levels of any one or more differentially expressed proteins and/or miRNAs can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold higher than that of a reference biosample. In an aspect, the number of “times” the level of one or more differentially expressed proteins and/or miRNAs is lower or higher than that of a reference level can be a relative or an absolute number of times. Or, in an aspect, the level of the proteins and/or miRNAs can be normalized to a standard and these normalized levels can then be compared to one another to determine whether the differentially expressed proteins and/or miRNAs is lower or higher.

In an aspect, administering can comprise titrating a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects. In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a therapeutically effective dose of the disclosed angiotensin II receptor antagonist can comprise about 0.01 mg/kg/day to about 200 mg/kg/day. In an aspect, a therapeutically effective dose of the disclosed angiotensin II receptor antagonist can comprise about 50 mg/kg/day to about 150 mg/kg/day. In an aspect, a therapeutically effective dose of the disclosed angiotensin II receptor antagonist can comprise about 100 mg/kg/day to about 200 mg/kg/day. In an aspect, a therapeutically effective dose of the disclosed angiotensin II receptor antagonist can comprise about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day.

In an aspect, a therapeutically effective dose of a disclosed TRPC4 antagonist can comprise about 0.01 mg/kg/day to about 200 mg/kg/day. In an aspect, a therapeutically effective dose of a disclosed TRPC4 antagonist can comprise about 1 mg/kg/day to about 20 mg/kg/day. In an aspect, a therapeutically effective dose of a disclosed TRPC4 antagonist can comprise about 50 mg/kg/day to about 150 mg/kg/day. In an aspect, a therapeutically effective dose of a disclosed TRPC4 antagonist can comprise about 100 mg/kg/day to about 200 mg/kg/day. In an aspect, a therapeutically effective dose of a disclosed TRPC4 antagonist can comprise about 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 110 mg/kg/day, about 120 mg/kg/day, about 130 mg/kg/day, about 140 mg/kg/day, about 150 mg/kg/day, about 160 mg/kg/day, about 170 mg/kg/day, about 180 mg/kg/day, about 190 mg/kg/day, or about 200 mg/kg/day. In an aspect, a therapeutically effective dose of a disclosed TRPC4 antagonist can comprise about 10 mg/kg/day.

In an aspect, a disclosed method of identifying the risk of aortic aneurysm and/or aortic dissection can further comprise increasing and/or decreasing the expression level of one or more aspects of the signaling cascade presented in FIG. 9.

In an aspect, a disclosed method of identifying the risk of aortic aneurysm and/or aortic dissection can further comprise (i) treating and/or preventing an aortic aneurysm, (ii) reducing the need for surgical intervention, (iii) slowing and/or inhibiting disease progression, (iv) treating and/or preventing hereditary aortopathy, or (v) any combination thereof.

In an aspect, a disclosed method can comprise repeating an administering step one or more times. For example, a disclosed TRPC4 antagonist can be administered one or more times. In an aspect, a disclosed angiotensin II receptor antagonist can be administered one or more times. In an aspect, a disclosed composition or pharmaceutical composition comprising a disclosed TRPC4 antagonist and/or a disclosed angiotensin II receptor antagonist can be administered one or more times. In an aspect, administering of any disclosed antagonist, disclosed composition, and/or disclosed pharmaceutical formulation can comprise intravenous administration or oral administration.

In an aspect, the number of surgeries required by the subject in need thereof can be reduced by 1 surgery, 2 surgeries, 3 surgeries, 4 surgeries, 5 surgeries, or more than 5 surgeries than a subject not being subjected to or exposed to a disclosed method. For example, a subject in thereof can be subjected to 1 fewer surgery, 2 fewer surgeries, 3 fewer surgeries, 4 fewer surgeries, 5 fewer surgeries, or more than a subject not being subjected to or exposed to a disclosed method. In an aspect, the number of surgeries required by the subject in need thereof can be reduced by 10%, 20%, 30%, 40%, 50%, 60%, 70%, 80%, 90%, or more than 90% when compared to a subject not being subjected to or exposed to a disclosed method. In an aspect, the number of surgeries required by the subject in need thereof can be reduced by 10%-30%, 30%-60%, 60%-90%, or more than 90% when compared to a subject not being subjected to or exposed to a disclosed method. In an aspect, a disclosed subject can be subjected to a disclosed method and not require additional surgeries.

In an aspect of a disclosed method, the number of surgeries required by the subject in need thereof can be reduced when compared to the number of surgeries required by a subject not receiving a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, a method of identifying a subject at risk of aortic aneurysm and/or aortic dissection can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both. In an aspect, modifying the treating step can comprise changing the amount of a disclosed angiotensin II receptor antagonist, the amount of a disclosed TRPC4 antagonist, or the amount of a disclosed composition comprising an angiotensin II receptor antagonist and a TRPC4 antagonist administered to the subject, changing the frequency of administration of a disclosed angiotensin II receptor antagonist, the amount of a disclosed TRPC4 antagonist, or the amount of a disclosed composition comprising an angiotensin II receptor antagonist and a TRPC4 antagonist, changing the route of administration of a disclosed angiotensin II receptor antagonist, the amount of a disclosed TRPC4 antagonist, or the amount of a disclosed composition comprising an angiotensin II receptor antagonist and a TRPC4 antagonist, or any combination thereof. In an aspect, modifying the administering step can comprise changing the amount of a disclosed angiotensin II receptor and/or a disclosed TRPC4 antagonist, a disclosed composition comprising a disclosed angiotensin II receptor and/or a disclosed TRPC4 antagonist.

In an aspect, a disclosed method of identifying a subject at risk of aortic aneurysm and/or aortic dissection can comprise improving and/or increasing the life expectancy of a disclosed subject or a disclosed patient. In an aspect of a disclosed method, the life expectancy of the recipient or subject in need thereof is increased. In an aspect, life expectancy can be increased by 5-10 years, by 10-15 years, by 15-20 years, by 20-25 years, by 25-30 years, by 30-35 years, by 35-40 years, or more than 40 years when compared to a subject not being subjected to or exposed to a disclosed method.

In an aspect, a disclosed method of identifying a subject at risk of aortic aneurysm and/or aortic dissection can further comprise improving the subject's hemodynamics. Hemodynamics are known to the skill in the art. In an aspect, hemodynamics can comprise blow flow in the aortic root, sinuses, coronary orifices, or any combination thereof.

In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects.

In an aspect, a disclosed method of identifying a subject at risk of aortic aneurysm and/or aortic dissection can further comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof in a specific or disclosed subject. In an aspect, a disclosed method can comprise titrating the dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof to identify an effective dose and/or to identify an effective dose eliciting only mild adverse and/or side effects for a specific or disclosed subject.

In an aspect, administering comprises administering to the subject the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof. In an aspect, administering comprises administering to the subject less than the maximum tolerated dose of a disclosed angiotensin II receptor antagonist and/or a disclosed TRPC4 antagonist, a disclosed composition comprising a disclosed angiotensin II receptor antagonist and/or a disclosed TRPC4 antagonist, or any combination thereof.

In an aspect, the growth of an aneurysm can be measured using echocardiography, imaging techniques (e.g., MRI and/or computed tomography), abdominal or pelvic ultrasonography, or any combination thereof. In an aspect, aortic aneurysm size can be a critical determinant of the need for intervention, yet the maximal diameter can often vary depending on the modality and method of measurement. In an aspect, a CT scan of a disclosed subject or disclosed patient can comprise using a three-dimensional reconstruction software. In an aspect, maximal aortic diameter can be recorded in the anteroposterior (CT-AP) plane, the maximal ellipse (CT-ME), perpendicular to the maximal ellipse (CT-PME), or perpendicular to the centerline of flow (CT-PCLF), or any combination thereof. In an aspect, a disclosed aneurysm can be analyzed according to the principles of Bland and Altman.

In an aspect, a disclosed method of identifying the risk of aortic aneurysm and/or aortic dissection can further comprise administering to the subject (i) one or more active agents, (ii) biologically active agents, (iii) one or more pharmaceutically active agents, (iv) one or more immune-based therapeutic agents, (v) one or more clinically approved agents, or (vi) a combination thereof.

### K. Miscellaneous

Disclosed herein is a method for the prevention of a hereditary aortopathy in a subject, the method comprising, consisting of, or consisting essentially of administering to the subject a therapeutically effective amount of an angiotensin II receptor antagonist, or a pharmaceutical composition thereof, and a TRPC4 antagonist, or a pharmaceutical composition thereof, such that the hereditary aortopathy is prevented in the subject. Disclosed herein is a method for the treatment of a hereditary aortopathy in a subject, the method comprising, consisting of, or consisting essentially of administering to the subject a therapeutically effective amount of an angiotensin II receptor antagonist, or a pharmaceutical composition thereof, and a TRPC4 antagonist, or a pharmaceutical composition thereof, such that the hereditary aortopathy is treated in the subject. Disclosed herein is a method for the inhibition of aneurysm formation is a subject suffering from a hereditary aortopathy, the method comprising, consisting of, or consisting essentially of administering to the subject a therapeutically effective amount of an angiotensin II receptor antagonist, or a pharmaceutical composition thereof, and a TRPC4 antagonist, or a pharmaceutical composition thereof, such that the aneurysm formation is inhibited in the subject/Disclosed herein is a method for the prevention of aneurysm formation is a subject suffering from a hereditary aortopathy, the method comprising, consisting of, or consisting essentially of administering to the subject a therapeutically effective amount of an angiotensin II receptor antagonist, or a pharmaceutical composition thereof, and a TRPC4 antagonist, or a pharmaceutical composition thereof, such that the aneurysm formation is prevented in the subject. In an aspect of a disclosed method, the angiotensin II receptor antagonist can be administered prior to the TRPC4 antagonist, administered concurrently with the TRPC4 antagonist, and/or administered after the TRPC4 antagonist. In an aspect of a disclosed method, a disclosed angiotensin II receptor antagonist can be Azilsartan (Edarbi), Candesartan (Atacand), Eprosartan, Irbesartan (Avapro), Losartan (Cozaar), Olmesartan (Benicar), Telmisartan (Micardis), Valsartan (Diovan), and any combinations thereof. In an aspect of a discloed method, a disclose angiotensin II receptor can comprise Losartan (Cozaar). In an aspect, a disclosed TRPC4 antagonist can be ML204, HC-070, and a combination thereof. In an aspect, a disclosed TRPC4 antagonist can comprise ML204.

### VII. EXAMPLES

MFS is an autosomal-dominant, connective tissue disorder that affects 50,000-70,000 patients in the United States (Robinson P N, et al. (2006) J Med Genet. 43:769-787; Judge D P, et al. (2005) Lancet. 366:1965-1976). It is characterized by mutations in fibrillin-1 (Fbnl), an extracellular anchoring glycoprotein required in the development of elastic and non-elastic tissues throughout the body (Hilhorst-Hofstee Y, et al. (2010) Hum Mutat. 31: E1915-1927). As a result of these mutations, patients with MFS experience an increase in downstream transforming growth factor beta (TGF-β and weaker vascular structures, particularly those in high pressure environments. Clinically, this results in several skeletal as well as cardiovascular features including disproportionately long limbs, skin striae, ectopia lentis, mitral valve prolapse, dilated cardiomyopathy, and ascending aortic aneurysms

Ascending aortic aneurysms are the leading cause of morbidity and mortality for patients with MFS. While the exact mechanism remains unknown, increased TGF43 and downstream extracellular signal-regulated kinase (ERK) ½ or c-Jun N-terminal kinase-1 (JNK-1) are known to be necessary (Holm T M, et al. (2011) Science. 332:358-361). (FIG. 9). Because angiotensin II also promotes ERK ½ through activity at angiotensin II receptor I, Losartan, a competitive antagonist at this receptor with anti-TGF-P properties, was initially explored as a novel therapy in MFS (Habashi J P, et al. (2006) Science. 312:117-121; Du J, et al. (2008) Am J Physiol Renal Physiol. 294: F909-918). Preclinical data showed that Losartan blocked aneurysm formation in MFS mice, although similar results could not be replicated clinically, wherein Losartan only slowed aneurysm progression and did not affect the number of patients who ultimately required surgery (Lacro R V, et al. (2014) N Engl J Med. 371 (22): 2061-2071).

Subsequent studies have shown that aneurysm size and the type of Fbnl mutation both influence patients' response to therapy. Briefly, while Losartan blocks aneurysm development in MFS mice who receive it prior to aneurysm development, it only attenuates aneurysm growth for mice started on therapy later in life, mirroring MFS clinically (Habashi J P, et al. (2006) Science. 312:117-121; Yang H H, et al. (2010) J Thorac Cardiovasc Surg. 140:305-312). Furthermore, while over 2900 Fbnl mutations have been described in MFS, they can be divided into two categories-(i) dominant negative mutations (DN, defined by the presence of both dysfunctional and functional Fbnl, which represents approximately 66% of patients), and (ii) haploinsufficient mutations (HI, defined by the presence of only functional Fbnl although at lower concentrations, 33% of patients) (Franken R, et al. (2014) Expert Opin Orphan Drugs. 2:1049-1062; Franken R, et al. (2015) Circ Cardiovasc Genet. 8:383-388; Aubart M, et al. (2015) Hum Mol Genet. 24:2764-2770). MFS patients with HI mutations experience a higher rate of aneurysm growth, dissection, and death than those with DN mutations, despite no differences in medical management (Franken R, et al. (2017) Heart. 103:1795-1799).

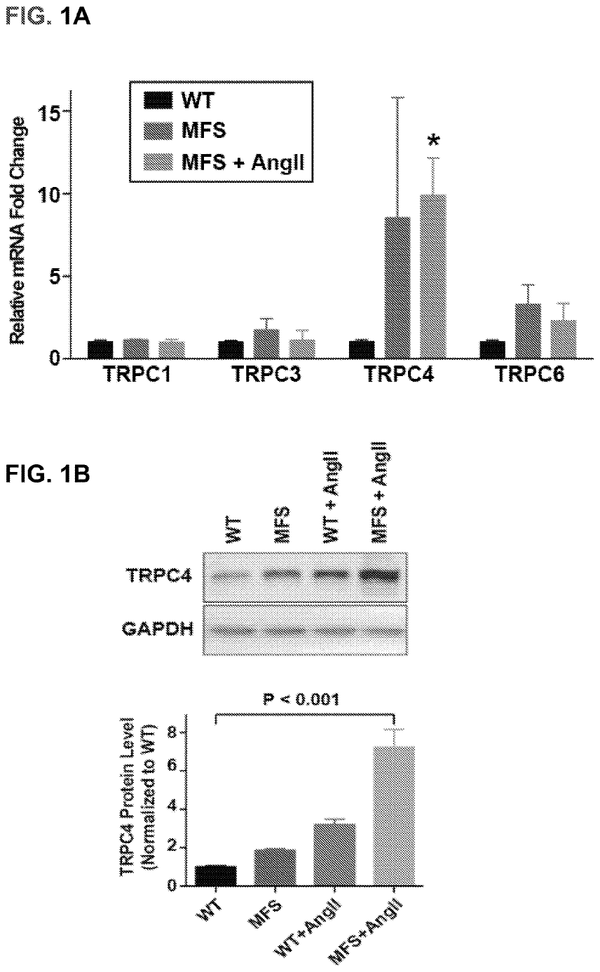

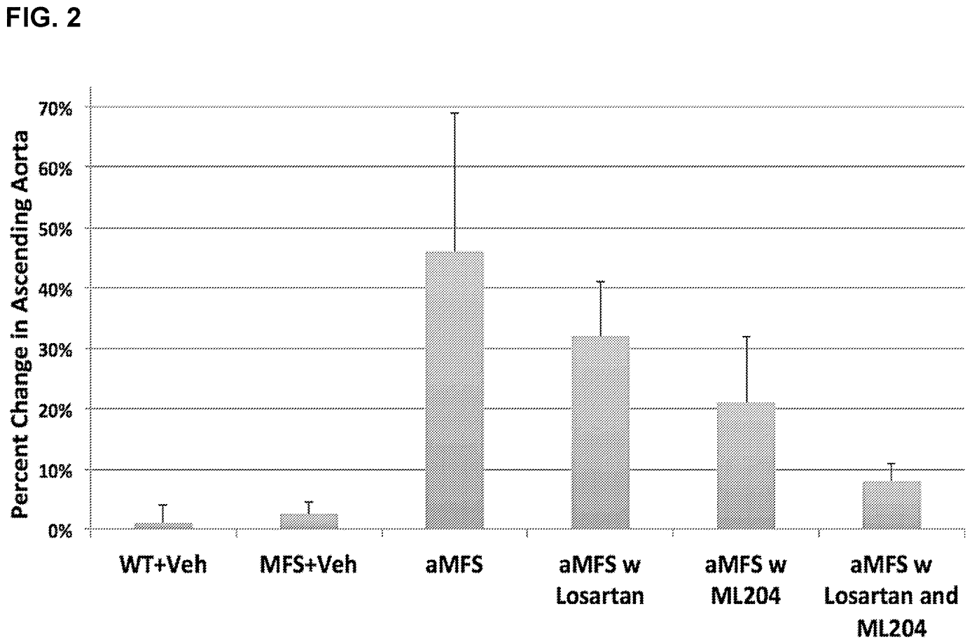

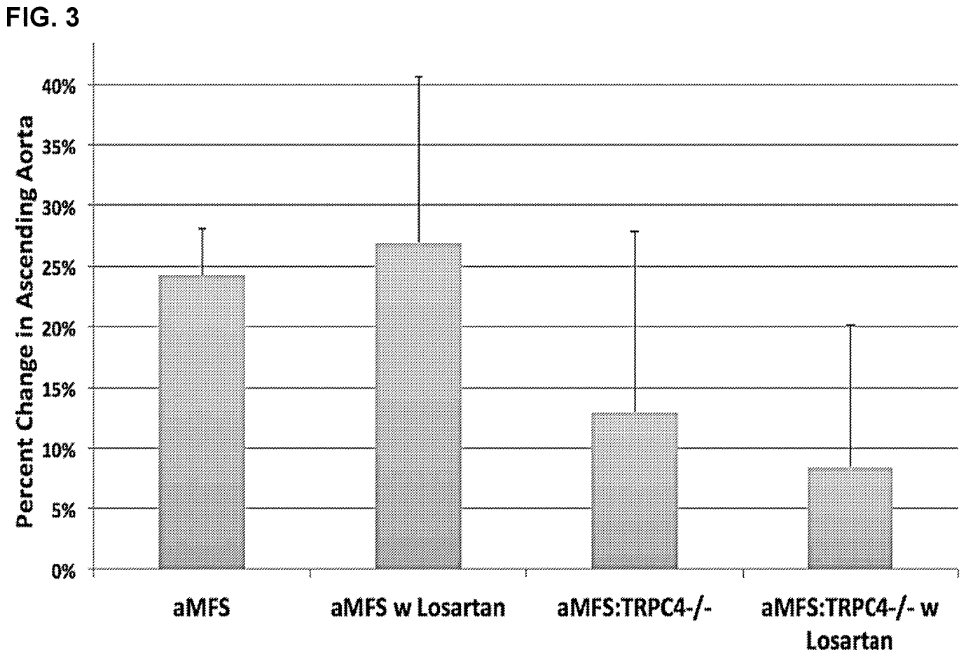

To better understand these clinical differences, a MFS mouse model was generated to facilitate high throughput therapeutic screening. Using exogenous angiotensin II in MFS mice, ascending aortic aneurysms can be generated in 2 weeks instead of 6-9 months (Cavanaugh N B, et al. (2017) Ann Thorac Surg. 104:657-665). These aneurysms develop through previously established MFS pathways, and like MFS clinically, only partially respond to Losartan therapy (Gensicke N M, et al. (2020) J Thorac Cardiovasc Surg. 159:1719-1726). Studies have demonstrated that TRPC4 was upregulated in MFS and played a role in aneurysm formation (FIG. 1-FIG. 4). By giving aMFS mice a TRPC4 antagonist and Losartan, aneurysm formation was prevented (FIG. 2). TRPC4 antagonists therefore represent an opportunity to replace surgery in MFS aortopathy.

As detailed in the specific Examples set forth below show, the mechanism by which TRPC4 potentiated aneurysm activity in MFS was characterized, candidate biomarkers related to aneurysm progression were explored, and a library of TRPC4 antagonists was developed.

### Example 1

### Characterization of Mechanism by which TRPC4 Potentiated Ongoing Aneurysm Activity in MFS

Ascending aortic aneurysms are the leading cause of morbidity and mortality for patients with Marfan syndrome (MFS). Current therapies slow, but do not stop aneurysm progression. The experiments described herein pursue 3 specific goals. First, the experiments characterize the mechanism through which TRPC4 potentiates ongoing aneurysm activity in MFS. Second, the experiments explore candidate biomarkers related to ascending aortic aneurysm activity. Third, the experiments design novel TRPC4 antagonists through structure-based computer-aided drug design.

Ascending aortic aneurysms are the leading cause of morbidity and mortality for patients with Marfan syndrome (MFS). Existing medical therapies slow aneurysm progression, but do not eliminate the need for surgery. As detailed in the specific Example below, transient receptor potential channel C4 (TRPC4) was upregulated in MFS and played a role in aneurysm formation. By administering a TRPC4 antagonist and Losartan, aneurysm progression in a traditionally refractory accelerated MFS (aMFS) mouse model was stopped. Clinically, ascending aortic aneurysms are established at the time of MFS diagnosis. The response of patients to medical therapy and their subsequent clinical course depends on the category of their underlying mutation and the size of their aneurysm at diagnosis. Earlier therapies in MFS failed to translate clinically, because they were started in models before aneurysms had formed.

Initiating Losartan in MFS mouse models after aneurysms have formed more accurately reflects MFS clinically, in that aneurysms continue to grow albeit at a slower rate. 129S6/SvEvTac (129S) MFS mice develop a more aggressive MFS phenotype than traditional C57BL/6J mice. Using 129S MFS mice, Example 1 does the following: (i) explores the biochemical profile of MFS aortopathy over time in haploinsufficient (HI, Fbnling^{Rk}) and dominant negative (DN,_{Fbni} C 1039G/+) MFS mouse models, (ii) evaluates the impact of TRPC4 inhibition, Losartan, and combination therapy on established aneurysms of various sizes in HI and DN MFS mice, and (iii) identifies the breakthrough biochemical profile within aneurysms for MFS patients undergoing elective repair.

TRPC4 was identified as a therapeutic target in MFS aortopathy using exogenous angiotensin II. (Cavanaugh N B, et al. (2017) Ann Thorac Surg. 104:657-665). Angiotensin II is a known promoter of TRPC4 (Matrougui K, et al. (2000) Hypertension. 36:617-621).

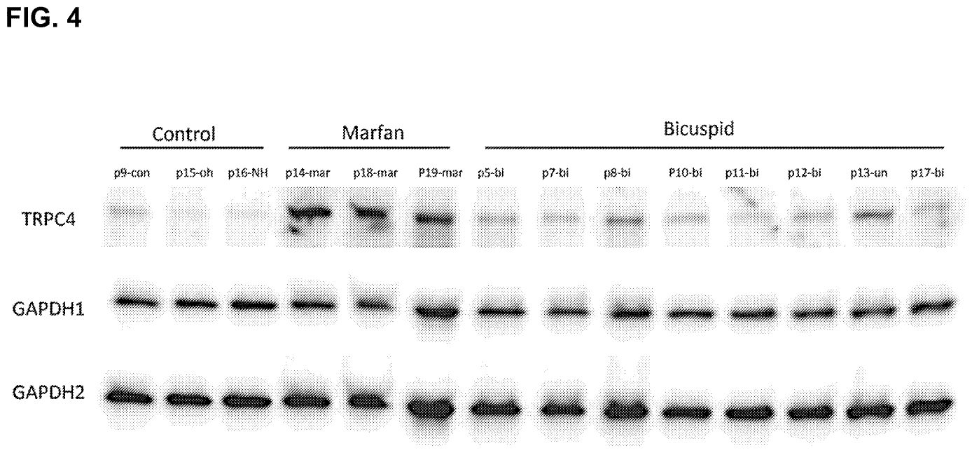

While TRPC4 is upregulated in systems without exogenous angiotensin II including the traditional MFS mouse model and patients clinically, replicating the findings in a more physiologic system is important (FIG. 1 and FIG. 4).

MFS mice started on Losartan at an early age did not develop aneurysms (Holm T M, et al. (2011) Science. 332:358-361). Although, if the treatment was delayed in these animals until after aneurysms have formed, then Losartan had less of an effect (Lacro R V, et al. (2014) New Engl J Med. 371 (22): 2061-2071; Yang H H, et al. (2010) J Thorac Cardiovasc Surg. 140:305-312).

Aneurysms continue to grow, albeit at a slower rate, which is similar to what is observed by the inventors in the clinic. This outcome provides with the opportunity to test TRPC4 antagonists in a more representative MFS model.

By using 129S MFS mice with HI and DN Fbnl mutations, the baseline differences in aneurysm development can be identified. Changes in the expression profiles due to therapy can be identified and tracked for for aneurysms of various sizes, and then compared to the findings to the breakthrough biochemical profiles for MFS patients (i.e., ongoing ERK1/2 or JNK-1 signaling despite treatment). The data from these experiments feed into the design of successful therapies for MFS patients. DN 129S MFS and wild-type mice are obtained from a collaborator while HI 129S MFS mice are obtained from Jackson Labs (Bar Harbor, ME). These experiments are performed with at least 170 DN 129S MFS mice (85 males/85 females), 170 HI 129S MFS mice (85 males/85 females), and 10 wild-type controls (5 males/5 females) (FIG. 6).

At birth, 15 DN MFS mice are sedated with inhaled anesthetic and undergo micro-computed tomography (micro-CT) imaging using available imaging techniques. For each animal, the aortic root diameter, aortic root cross-sectional area, left ventricular volume, lung architecture, and degree of kyphosis are measured. Following imaging, 10 mice are assigned to Losartan (0.6 g/L delivered via drinking water) and the other 5 mice are sacrificed as biochemical controls to evaluate TRPC4, MMPs, and downstream TGF43 signaling in their aortic roots at birth. For these mice, blood is collected by cardiac puncture (approx. 1.5 mL) and stored in RNA safe micro-centrifuge tubes. Representative samples of the ascending aorta (1 mm-1.5 mm), heart, and lungs are preserved as a formalin fixed, paraffin embedded (1⁴1-4VE) specimens. The remaining ascending aorta is flash frozen in liquid nitrogen for homogenization and western blot analysis.

At 2 months of age, 35 more untreated MFS mice are imaged. Following imaging, 10 mice are randomly assigned to receive Losartan therapy, 10 mice assigned to receive TRPC4 antagonist therapy (10 mg/kg/day ML204, AdooQ Bioscience, Irvine, CA) via subcutaneous osmotic mini-pump placement, and 10 mice are assigned to receive combination therapy (both treatments). The remaining 5 mice in the 2-month enrollment cohort are sacrificed as biochemical controls.

At 4, 6, and 8 months, this process is repeated with 35 additional untreated DN MFS mice being enrolled each time, imaged, and randomly assigned to treatment categories (i.e., biochemical control, Losartan, ML2014, or combination therapy). Five wild-type 129S mice are also be imaged, sacrificed, and cataloged as previously described at birth and 8 months of age (serving as a second control). All treated mice are imaged at 6 months and 12 months of age. This experiment concludes when 50% of mice within a treatment arm die or 12 months is reached, whichever occurs first. At that time, mice are imaged, sacrificed, and cataloged. The same study design is implemented for HI 129S MFS mice as well.

The wall architecture and protein expression within aortic sections are characterized following previously described methods. Briefly, 141-4VE specimens are cut to provide 5-micron perpendicular cross sections of the aorta. Representative sections are used to measure wall thickness, elastic fiber architecture, collagen deposition, and phosphorylated Smad2 localization.

Protein is extracted from flash-frozen and homogenized aortic samples for western blot analysis against known markers of MFS aortopathy including Smad2, Smad3, JNK1, ERK1/2, ERK5, MEK1, ROCK1, and p38, as well as MMP2, MMP9, MMP17, and TRPC4.

Representative aneurysm samples are collected from MFS patients undergoing elective aneurysm repair. Protein expression is analyzed for evidence of a breakthrough biochemical profile (i.e., residual TGF-β, MMP, or TRPC4 signaling despite medical management).

Patient information regarding age, sex, preceding interval aneurysm growth, medications prescribed, and information on the specific-type of Fbnl mutation is stored with the tissue—The experiments set forth herein are powered to detect at least a 33% reduction in aortic diameter growth for patients on combination therapy versus Losartan alone (assuming 0.4 mm growth for Losartan with 0.1 mm standard deviation, beta of 0.8, and an alpha of 0.05). If there is more variability or error around Losartan (0.2 mm instead of 0.1 mm), then the use of 10 mice per arm detects at least a 63% reduction in aortic diameter growth between the two cohorts. Experience indicates 0.4 mm growth with 0.1 mm standard deviation on Losartan is reasonable and likely estimate (ng H H, et al. (2010) J Thorac Cardiovasc Surg. 140:305-312). Furthermore, foundational data showed a 75% reduction in aortic diameter growth between mice on combination therapy and Losartan, ensuring that the study is powered appropriately (FIG. 2).

Based on the data, wild-type 129S aorta roots measure between 1.5 mm and 1.8 mm in size. At 2 and 6 months, DN 129S MFS aortic roots measured 2.2 mm and 3 mm, respectively. Therefore, root diameters of 1.8 mm, 2.2 mm, 2.6 mm, 3.0 mm, and 3.2 mm are anticipated in 129S MFS mice at 0 months, 2 months, 4 months, 6 months, and 8 months, respectively. By 8 months, about 40% of the untreated MFS mice are expected to experience dissection.

Mice started on Losartan at birth are not expected to experience aneurysm growth. HI mice experience a higher rate of dissection than DN MFS mice. And those being started on single therapies (Losartan or ML204) at 6 months and 8 months are also expected to experience dissection, although to a lesser degree and later than those left untreated. Mice that are started on combination therapy before 6 months are not expected to experience dissection. The rate of residual growth despite therapy is expected to correlate with the amount of ERK1/2 within the aneurysm at the end of the study or at the time of repair.

The primary outcome for this series of experiments is a change in diameter or cross-sectional area of the aortic root after the initiation of Losartan, ML204, or the combination of both Losartan and ML204. Change in aneurysm size are calculated by finding the difference in measurements between the time therapy was started and the end of the protocol.

Pairwise Student's t-tests are used to compare the differential effect of treatments that were started at the same time (e.g., the difference between therapy with Losartan, with ML204, or with the combination of Losartan and ML204 for mice in the 2 month or 4-month cohort). The percent reduction in ERK1/2 levels is calculated within treatment groups using biochemical control animals as the associated reference value. Pairwise Student's t-tests are used. If the variables are not normal, then instead of pairwise Students t-tests, the Wilcoxon Signed Rank test is used. When more than 2 time points are available, the repeated measures data are also be analyzed using linear mixed models. Kaplan Meier survival analysis are also used to evaluate freedom from dissection between HI and DN mice as well as differences in dissection rates among treatments.

### Example 2

### Exploration of Candidate Biomarkers Related to Ascending Aortic Aneurysm Activity

The work described as part of this example explores candidate biomarkers related to ascending aortic aneurysm activity. A peripheral biomarker of aneurysm activity has long been sought by the skilled person in the art. Circular and microRNA have been implicated in phenotypic switching within vascular smooth muscle cells during aneurysm development. Xu et al. showed that circular TGF43 receptor 2 (circTGFB2R) attenuates microRNA 29a (miR-29a) activity to prevent phenotypic switching, and therefore aneurysm development, in human aortic vascular smooth muscle cells. (Xu Z, et al. (2021) J Inflamm Res. 4:5877-5890).

Using 129S MFS mice, Example 2 uses quantitative PCR to detect differences in peripheral circTGFB2 and miR-29a levels between non-aneurysmal controls (wild-type), untreated MFS mice with aneurysms, partially treated MFS mice with aneurysms (Losartan), and fully treated MFS mice with aneurysms (TRPC4 antagonist and Losartan). Example 2 also uses alternative biomarkers associated with aneurysm activity through microarray analysis. Further, Example 2 validates biomarker expression with respect to change in aneurysm size for MFS patients followed clinically.

As stated above, a peripheral biomarker of aneurysm activity in MFS provides insight into the effectiveness of medical management, identify high-risk patients earlier, and minimize the need for serial imaging in those patients with well controlled disease. Identification of a biomarker has historically been limited by technology and the lack of understanding of what to look for. Circular and microRNA have been implicated in phenotypic switching within vascular smooth muscle cells during aneurysm development. Xu et al. showed that circular TGFβ receptor 2 (circTGFB2R) attenuates microRNA 29a (miR-29a) activity to prevent phenotypic switching, and therefore aneurysm development, in human aortic vascular smooth muscle cells (Xu et al. (2021) J Inflamm Res. 4:5877-5890). MiR-29a and other noncoding RNAs can be detected in the peripheral circulation and represent a previously unexplored category of potential biomarkers in MFS aortopathy.

In this Example, blood from 129S wild-type controls, untreated MFS mice, partially treated MFS mice (Losartan), and fully treated MFS mice (ML204 and Losartan) is obtained as detailed above in Example 1. Blood from mice within the same treatment cohort are pooled in RNA safe collection tubes. RNA is extracted from 1 mL of whole blood within each respective sample using a blood total RNA extraction kit (RiboPure, ThermoFisher, Waltham, MA). The extracted RNA concentration is measured using a Nanodrop spectrophotometer, and its integrity using gel electrophoresis. RNA samples with concentrations>200 ng/μL and an A260/A280 between 1.8 and 2.0 are used for reverse transcription reactions. Quantitative PCR is performed on an ABI PRISM analyzer with 5 ng of cDNA, 5 μL of TaqMan solution, 3 μL of RNase-free water, and 1 μL of primers (forward and reverse) at 95° C. for 3 minutes followed by 40 amplification cycles (95° C. for 10 seconds and 60° C. for 30 seconds). GAPDH is used to normalize the values. These experiments use microarray analysis to verify the experiments described supra and explore alternative biomarkers of aneurysm activity within MFS. Briefly, RNA is extracted from whole blood samples from wild-type mice, untreated, partially treated, and fully treated MFS mice with established aneurysms. RNA concentrations and integrity are again tested using a Nanodrop spectrophotometer and gel electrophoresis. Digestion, amplification, and labeling are performed according to the manufacturer's protocol. Labeled RNA is hybridized onto the circular (Mouse circRNA array, Arraystar Inc, Rockville, MD) and microRNA microarrays (TaqMan Rodent MicroRNA A Array 2.0, ThermoFisher, Waltham, MA). Each microarray can interrogate over 14,000 unique circular and 350 unique microRNA constructs.

Primers are made for differentially expressed circular or microRNA with over 2-fold change between untreated and fully treated MFS mice. Four (4) mL of whole blood are collected in RNA safe centrifuge tubes from MFS patients undergoing elective aneurysm repair in addition to the tissue collected as described supra. RNA is extracted from the whole blood to make cDNA and to evaluate candidate biomarkers with qPCR. Associated tissue samples from the excised aneurysms are also tested for the same biomarkers to provide a correlation between expression levels at the site of activity and those biomarkers peripherally obtained. Biomarker levels are correlated to the most recent change in the aneurysm size of the patients based on annual imaging.

Because CircTGFB2R attenuates miR-29a to prevent the ascending aortic aneurysm phenotype, an abundance of miR-29a is expected in untreated MFS aortopathy. Differential microarray analysis demonstrates a number of candidate biomarkers exists on a continuum across the untreated, partially treated, and fully treated phenotypes. Together with miR-29a, these biomarkers are used to generate a highly sensitive activity profile for MFS aortopathy that is clinically relevant.

Quantitative PCR results are calculated using double delta cycle threshold analysis. Microarray analyses are analyzed collaboratively. Wild-type samples serve as a control and are initially compared against samples from untreated MFS mice with established aneurysms. Candidate biomarkers, microRNA, and circular RNA with greater than 2-fold variation between these two populations are then assessed in partially treated and fully treated MFS mice to evaluate for a return to baseline (wild-type levels). Differences in expression levels between treatment groups are compared using Student's t-tests. The diagnostic value of a given candidate biomarker is verified using a receiver operator curve analysis. The relationship between candidate biomarkers and aneurysm growth (activity) is evaluated using Pearson correlation. Final logistic regression considers a patient's age, sex, aneurysm size, biomarker levels, and Fbnl mutation category (HI or DN) if available. Model assumptions are examined, and models are not overfit.

The use of microarrays may lead to a large number of candidates RNAs. These are narrowed based on trends observed between wild-type, untreated, partially treated, and completely treated animals. Furthermore, target transcripts for candidate microRNAs are examined against the known pathophysiology of aneurysms. qPCR probes are generated for the top 10-20 noncoding RNA biomarker candidates.

### Example 3

### Identification of TRPC4 Antagonists Through Structure-Based Computer-Aided Drug Design

The Examples provided herein characterize the identification of TRPC4 as a therapeutic target in MFS aortopathy, exploit the development of a more aggressive MFS model, and interrogate the potential role of noncoding RNA as a peripheral biomarker of aneurysm activity. The MFS phenotype exists on a continuum. By using exogenous angiotensin II, the MFS background was rapidly exposed and TRPC4 was identified as a therapeutic target (Cavanaugh N B, et al. (2017) Ann Thorac Surg. 104:657-665).

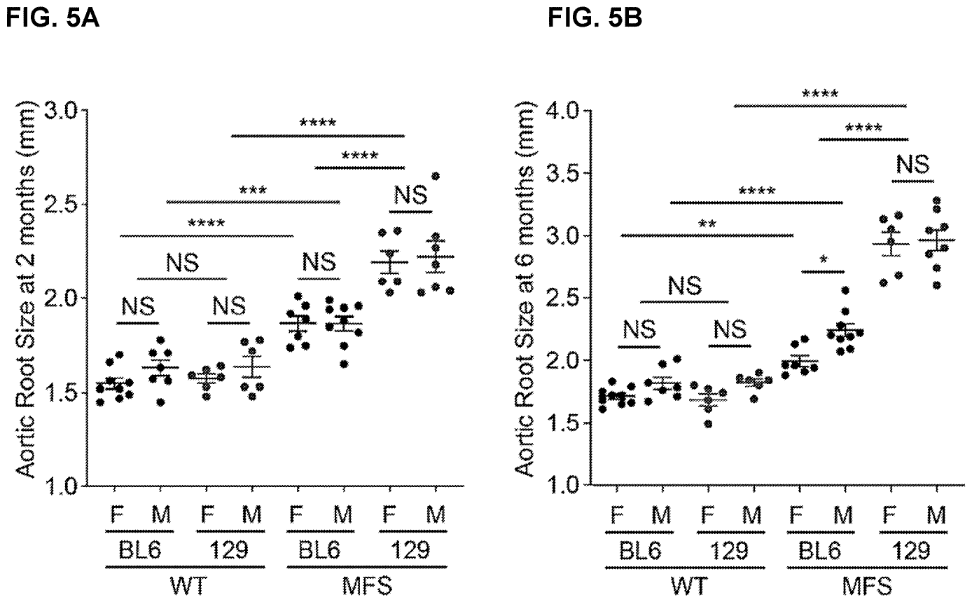

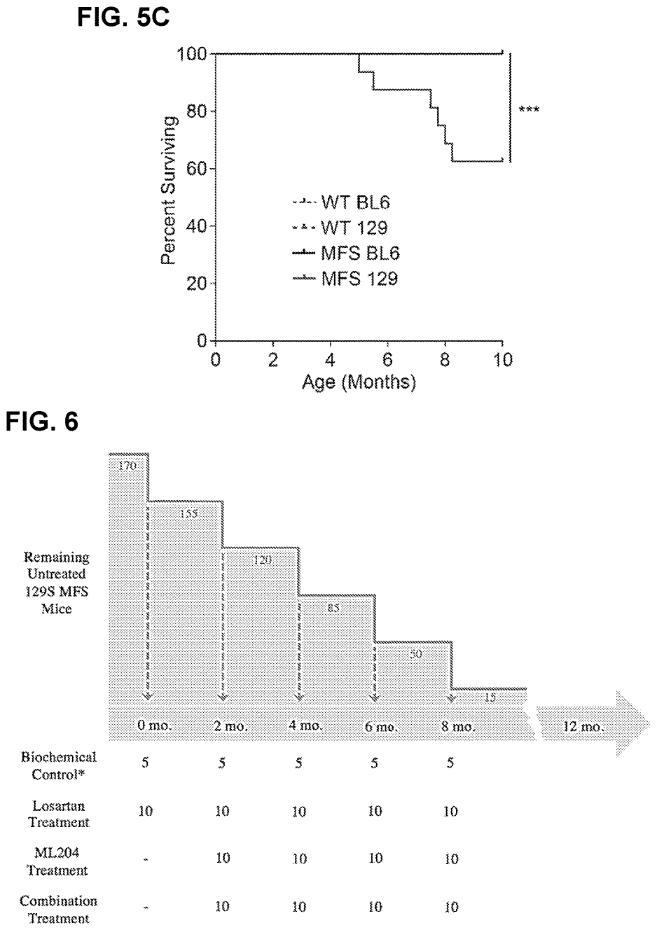

MFS mice developed on a 12956/SvEvTac (129S) background develop more aggressive aortic aneurysms than traditional C57BL/6J mice. By 2 months, 129S mice develop aneurysms, and by 8 months 40% of untreated mice experience dissection (FIG. 5). Genome-wide linkage analysis showed that restoration of normal Mmpl7 and Map2k6 activity were responsible for the more aggressive phenotype. In addition to its proteolytic activity, Mmpl7 also promotes aneurysm formation through direct interactions with EGFR and the p38MAPK/ERK pathway (Paye A, et al. (2014) Cancer Res. 23:6758-6770). Erolotinib, an EGFR antagonist, was shown to block these effects and rescue the aggressive MFS phenotype. In separate studies, TRPC4 inhibition was shown to block similar downstream VEGF/p38MAPK/ERK pathways (Song H B, et al. (2015) Cell Calcium. 57:101-108). Together these results support an association between TRPC4 and MFS aortopathy and the utility of 129S mice in this study.

TRPC4 makes sense as a therapeutic target in MFS aortopathy. As a membrane-bound ion channel, TRPC4 associates with the underlying cytoskeleton to regulate intracellular calcium and endothelial permeability in response to physical (e.g., stretch) and chemical (e.g., intracellular calcium) stimuli (Cioffi D L, et al. (2006) Microcirculation. 13:709-723). It localizes with other TRP channels and cadherins in the pulmonary arteries, cardiomyocytes, ascending aorta, intestine, and kidneys to carry out these functions. TRPC4 has also been associated with pathological processes like right heart failure, Crohn's, and glomerular sclerosis, wherein increased activity—at times through TGFβ signaling—has been shown to drive phenotypic switching, myoblast activation, actin stress fiber formation, fibrosis, and pathological remodeling (Kurahara L H, et al. (2016) J Smooth Muscle Res. 52:78-92; Wes P D, et al. (1995) Proc Natl Acad Sci USA. 92:9652-9656; Tamaki K, et al. (1994) Kidney Int. 45 (2): 525-536; Camacho Londoño J E, et al. (2015) Eur Heart J. 36:2257-2266; Sabourin J, et al. (2018) J Mol Cell Cardiol. 118:208—

There is increasing evidence that noncoding RNA serves as a biomarker of systemic processes (Zhao N, et al. (2015) Circ Res. 116:23-34; Kang H, et al. (2012) Curr Opin Hematol. 19:224-231; Hang D, et al. (2018) Cancer Medicine. 7:2783-2791; Lin J, et al. (2019) Clinical Biochemistry. 74:60-68; Zhao Z, et al. (2017) Sci Rep. 7:39918; Xu Z, et al. (2021) J Inflamm Res. 4:5877-5890). MicroRNA works by binding to and silencing active gene transcripts within a system. These complimentary transcripts work as a single long construct, or more commonly as a combination of shorter constructs, to fine tune gene expression with regional specificity. Recently, advances in sequencing and bioinformatics have shown that unique microRNA profiles can be used as biomarkers for systemic diseases including lung cancer, coronary artery disease, and colorectal cancer (Hang D, et al. (2018) Cancer Medicine. 7:2783-2791; Lin J, et al. (2019) Clinical Biochemistry. 74:60-68; Zhao Z, et al. (2017) Sci Rep. 7:39918). In a 2021 paper, Xu et al. showed that noncoding circular TGFβ receptor 2 (circTGFB2R) constructs sponge microRNA 29a (miR-29a) in vascular smooth muscle cells to prevent ascending aortic aneurysm development (Xu Z, et al. (2021) J Inflamm Res. 4:5877-5890). Quantitative PCR and microarray analysis represent proven approaches for detecting candidate biomarkers of MFS aortopathy and monitoring a patient's therapeutic response to treatment (Hang D, et al. (2018) Cancer Medicine. 7:2783-2791; Lin J, et al. (2019) Clinical Biochemistry. 74:60-68; Zhao Z, et al. (2017) Sci Rep. 7:39918; Xu Z, et al. (2021) J Inflamm Res. 4:5877-5890).

The commercial availability of TRPC4 antagonists is limited and none are currently FDA approved for human use. The limitations impact the ability to perform a clinical trial in MFS aortopathy. A single TRPC4 antagonist (BI 1358894), developed by Hydra Bioscience and tested in partnership with Boehringer Ingelheim, is currently in phase-2 clinical trials for the treatment of anxiety, proving early safety in humans.

Using previously published structural and sequence data on TRPC4, Open Source Protein Redesign for You (OSPREY 3.0) is first used to identify a list of novel TRPC4 antagonists. Then, the novel TRPC4 antagonists are synthesized and subjected to binding affinity assays. Third, following the binding affinity assays, promising therapeutic candidates are used in conjunction with the accelerated MFS mouse model described herein. In these experiments, ML204 is used as one of the controls or references. Finally, building on the experiments set forth above, the inventors file investigational new drug applications with the Food and Drug Administration for drugs that successfully block aneurysm development in combination with Losartan.

Historically, ion channel agonist and antagonists were identified by screening thousands of small-molecule libraries. While successful, this process took a significant amount of time and resources, candidate structures were limited to what was available in existing libraries, and a majority of proteins were considered undruggable due to the lack of small molecule binding sites.

Today, for well described channels, like TRPC4, this can be done virtually, allowing for the rapid identification of novel, potentially never-before synthesized solutions. The examples set forth herein use one of the first open-source structure-based, computer-aided drug discovery platforms (OSPREY, Open Source Protein Redesign for You). Today, this platform is in its third version and has been successfully used to identify novel therapeutic enzymes, drugs, peptide inhibitors, and antibodies (Hallen M A, et al. (2018) J Comput Chem. 39:2494-2507; Lowegard A U, et al. (2020) PLOS Comput Biol. 16: e1007447; Roberts K R, et al. (2012) PLOS Comput Biol. 4: e1002477). OSPREY 3.0 incorporates target structure, predicted energy, sequence space, and rotational flexibility to rapidly conduct a series of dead-end elimination algorithms and produce an ordered list of high-affinity, low-energy solutions. This program enables the rapidl identification and characterization of a series of stable, high-affinity, TRPC4 antagonists for clinical use in MFS aortopathy.

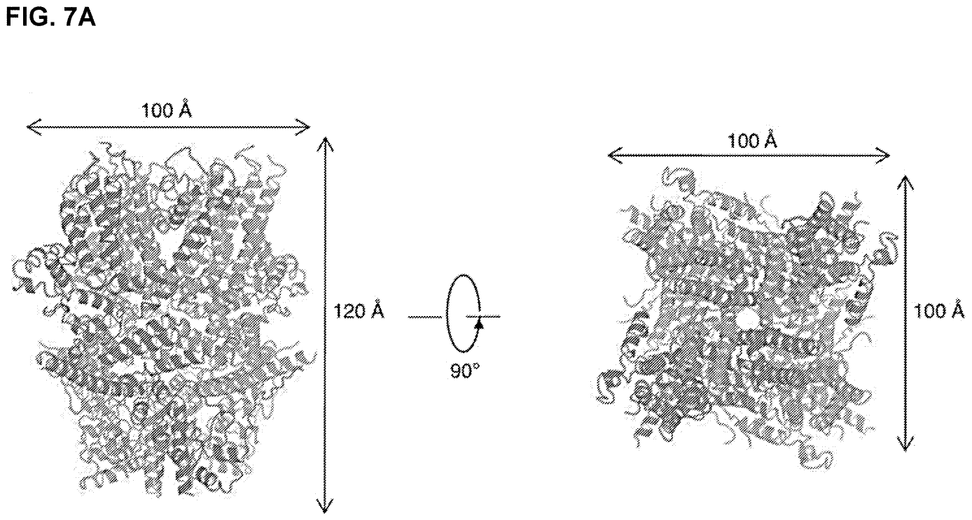

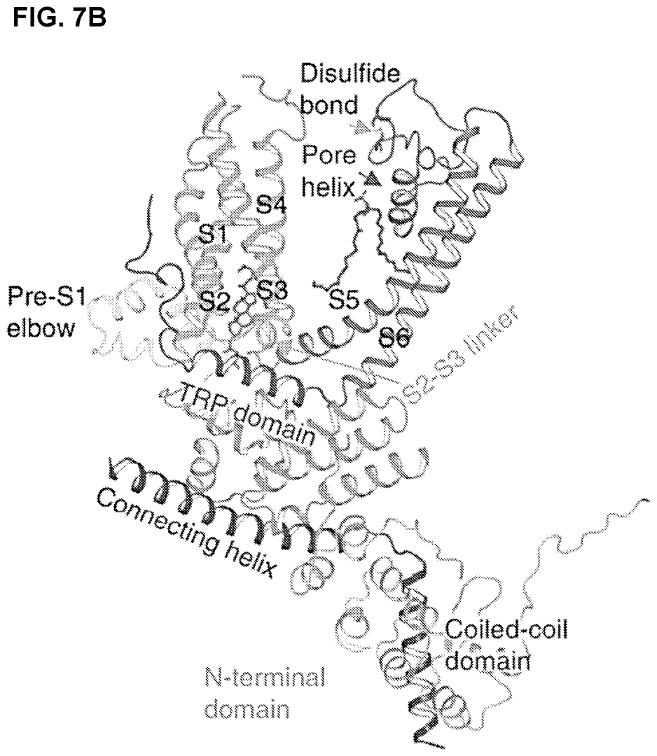

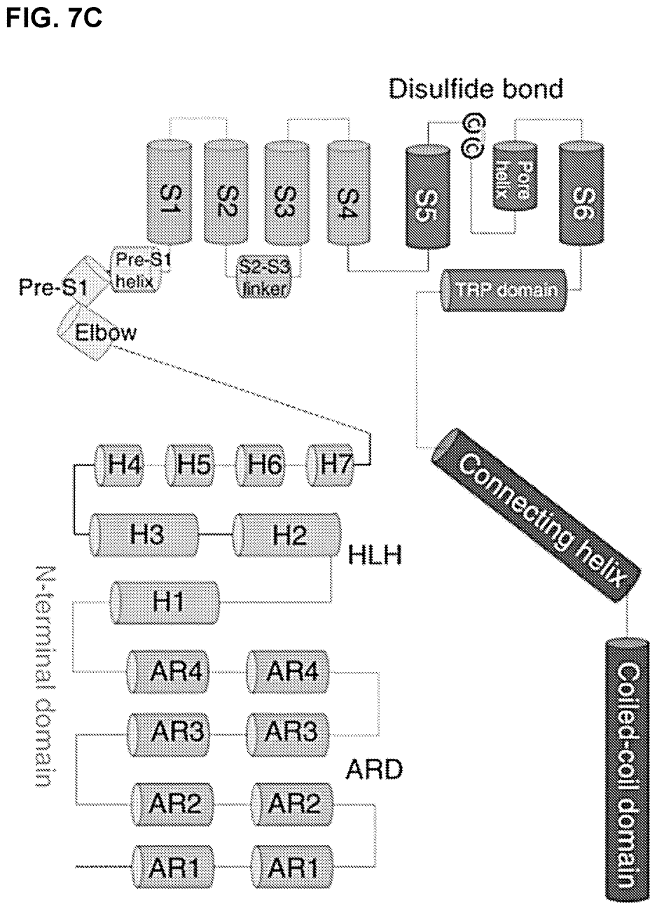

Sequence and structural knowledge of TRPC4 (PDB 7B16, 7B0S, 7B0J, 7B05, 6G1K, and 7B1G) is incorporated into OSPREY 3.0 (FIG. 7) (Duan J, et al. (2018) Nat Commun. 9:3102).

Alphafold2 is used to validate candidate structures. Peripheral regions of the channel (residues 677-692) are interrogated initially, given calmodulin's ability to restrict side-chain flexibility and inhibit channel activity at these sites. Extracellular targets associated with competitive, piperazinone/pyridazinone-based inhibition at the voltage-sensor like domain are also be explored (Vinayagam D, et al. (2020) eLife. 9: e60603).

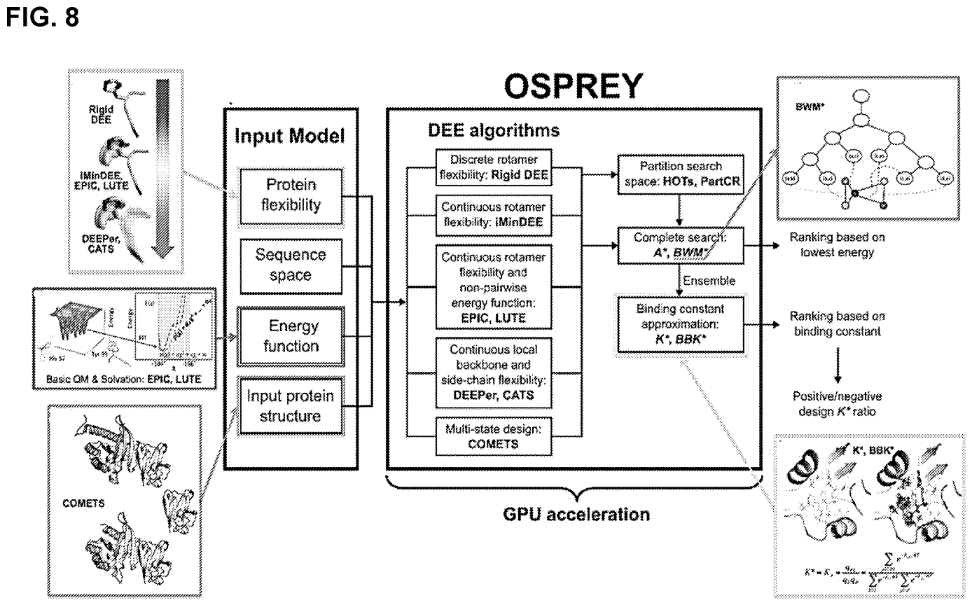

Synthetic small molecule, protein-protein, and peptidomimetic candidates aare then generated using Branch and Bound over K* (BBK*), Constrained Optimization of Multistate Energies by Tree Search (COMETS), Coordinate of Atoms by Taylor Series (CATS), Energy as Polynomials in Internal Coordinates (EPIC), and Dead-end Elimination with Perturbations (DEEPer) algorithms (FIG. 8). These algorithms incorporate both positive (ability to bind a specific target) and negative (ability to avoid binding unintended targets) design considerations, to avoid unintended interactions with other TRP channels. Resulting solutions arebe ranked based on target affinity and minimum energy conformation of the design. Candidate inhibitors with similar, if not greater, projected binding affinity than ML204 (IC₅₀ values of 0.96-2.6 μM) are synthesized.

Candidate compound binding affinity for TRPC4 ARE tested using a previously published protocol (see, e.g., Miller M, et al. (2011) Membrane Bio. 38:33436-33446). Briefly, stock HEK293 cells transfected with TRPC4 and u-opioid receptors are obtained. Cell stocks are expanded and loaded with Fluo4-AM for 45 minutes at room temperature. Cells are then washed and added to 384-well assay plate. Candidate compounds are added to the wells, allowed to incubate for a set period, then challenged with a known activator of the μ-opioid G coupled-protein receptor (DAMGO, [D-Ala², N-MdPhe⁴, Gly-ol]-enkephalin). Fluorescent changes, signaling an influx of calcium, are recorded on a kinetic imaging plate reader.

Candidate drugs with the highest TRPC4 affinity are also be tested in 129S MFS mice. Similar to Example 1, 129S mice are allowed to develop aneurysms and at 4 months of age randomized to receive no therapy, Losartan, ML204, Candidate Drug 1, Combination 1 (Losartan/ML204), or Combination 2 (Losartan/Candidate Drug).

All mice have their aortic roots measured at the time of enrollment using micro-CT. When 50% of the untreated MFS mice succumb to dissection, the study is terminated. All surviving mice are reimaged, euthanized, and processed for histology and protein-based analysis as described supra in Example 1. An investigational new drug application can be filed with the FDA for stable drug candidates with high TRPC4 affinity and proven effect in the 129S MFS mouse model. There are two known TRPC4 antagonists (ML204 and BI 1358894). Example 3 is designed to identify at least 2-4 stable antagonists with at least the same affinity as ML204 that block ongoing aneurysm activity when used with Losartan in 129S MFS mice. Novel TRPC4 antagonists are ranked on minimum energy conformation (DEE/A* algorithms) and on estimated target affinity (DEE/K* algorithms), which have been described and validated as described in Roberts K E, et al. (2015) Proteins. 10:1859-1877 and Lilien R H, et al. (2005) J Comput Biol. 12:740-761. Synthesized candidate affinities for TRPC4 are calculated by determining the final concentration of the drug candidate necessary to reduce maximum calcium influx (baseline fluorescence following DAMGO without an antagonist present) by 50% (IC₅₀). The endpoint of the animal studies is change in aortic root diameter or cross-sectional area. Student's t-test is used to compare these changes between treatment cohorts.

### SUMMARY

The Examples provided herein characterize the identification of TRPC4 as a therapeutic target in MFS aortopathy, exploit the development of a more aggressive MFS model, and interrogate the potential role of noncoding RNA as a peripheral biomarker of aneurysm activity. The MFS phenotype exists on a continuum. By using exogenous angiotensin II, the MFS background was rapidly exposed and TRPC4 was identified as a therapeutic target (Cavanaugh N B, et al. (2017) Ann Thorac Surg. 104:657-665).

MFS mice developed on a 12956/SvEvTac (129S) background develop more aggressive aortic aneurysms than traditional C57BL/6J mice. By 2 months, 129S mice develop aneurysms, and by 8 months 40% of untreated mice experience dissection (FIG. 5).

Genome-wide linkage analysis showed that restoration of normal Mmpl7 and Map2k6 activity were responsible for the more aggressive phenotype. In addition to its proteolytic activity, Mmpl7 also promotes aneurysm formation through direct interactions with EGFR and the p38MAPK/ERK pathway (Paye A, et al. (2014) Cancer Res. 23:6758-6770). Erolotinib, an EGFR antagonist, was shown to block these effects and rescue the aggressive MFS phenotype. In separate studies, TRPC4 inhibition was shown to block similar downstream VEGF/p38MAPK/ERK pathways (Song H B, et al. (2015) Cell Calcium. 57:101-108). Together these results support an association between TRPC4 and MFS aortopathy and the utility of 129S mice in this study.

TRPC4 makes sense as a therapeutic target in MFS aortopathy. As a membrane-bound ion channel, TRPC4 associates with the underlying cytoskeleton to regulate intracellular calcium and endothelial permeability in response to physical (e.g., stretch) and chemical (e.g., intracellular calcium) stimuli (Cioffi D L, et al. (2006) Microcirculation. 13:709-723). It localizes with other TRP channels and cadherins in the pulmonary arteries, cardiomyocytes, ascending aorta, intestine, and kidneys to carry out these functions. TRPC4 has also been associated with pathological processes like right heart failure, Crohn's, and glomerular sclerosis, wherein increased activity—at times through TGFβ signaling—has been shown to drive phenotypic switching, myoblast activation, actin stress fiber formation, fibrosis, and pathological remodeling (Kurahara L H, et al. (2016) J Smooth Muscle Res. 52:78-92; Wes P D, et al. (1995) Proc Natl Acad Sci USA. 92:9652-9656; Tamaki K, et al. (1994) Kidney Int. 45 (2): 525-536; Camacho Londoño J E, et al. (2015) Eur Heart J. 36:2257-2266; Sabourin J, et al. (2018) J Mol Cell Cardiol. 118:208—

There is increasing evidence that noncoding RNA serves as a biomarker of systemic processes (Zhao N, et al. (2015) Circ Res. 116:23-34; Kang H, et al. (2012) Curr Opin Hematol. 19:224-231; Hang D, et al. (2018) Cancer Medicine. 7:2783-2791; Lin J, et al. (2019) Clinical Biochemistry. 74:60-68; Zhao Z, et al. (2017) Sci Rep. 7:39918; Xu Z, et al. (2021) J Inflamm Res. 4:5877-5890). MicroRNA works by binding to and silencing active gene transcripts within a system. These complimentary transcripts work as a single long construct, or more commonly as a combination of shorter constructs, to fine tune gene expression with regional specificity. Recently, advances in sequencing and bioinformatics have shown that unique microRNA profiles can be used as biomarkers for systemic diseases including lung cancer, coronary artery disease, and colorectal cancer (Hang D, et al. (2018) Cancer Medicine. 7:2783-2791; Lin J, et al. (2019) Clinical Biochemistry. 74:60-68; Zhao Z, et al. (2017) Sci Rep. 7:39918). In a 2021 paper, Xu et al. showed that noncoding circular TGFβ receptor 2 (circTGFB2R) constructs sponge microRNA 29a (miR-29a) in vascular smooth muscle cells to prevent ascending aortic aneurysm development (Xu Z, et al. (2021) J Inflamm Res. 4:5877-5890). Quantitative PCR and microarray analysis represent proven approaches for detecting candidate biomarkers of MFS aortopathy and monitoring a patient's therapeutic response to treatment (Hang D, et al. (2018) Cancer Medicine. 7:2783-2791; Lin J, et al. (2019) Clinical Biochemistry. 74:60-68; Zhao Z, et al. (2017) Sci Rep. 7:39918; Xu Z, et al. (2021) J Inflamm Res. 4:5877-5890).

The examples provided herein remains the only work that has implicated TRPC4 involvement in the pathogenesis of Marfan syndrome or hereditary aortopathy related aneurysm formation. Not only does this work implicate TRPC4 antagonism, but also demonstrated that it was upregulated in the accelerated Marfan (aMFS) mouse model, worked synergistically with current ARB blockade (for example, losartan) to prevent aneurysms (using both TRPC4 blocker or TRPC4 knockout mice) and can be found in abundance (15× upregulation) in the aneurysm sacs of Marfan patients undergoing surgical repair. This work provides a treatment course that meets a long-felt need for successful, non-surgical treatment for MFS subjects.

TABLE 1
Listing of Sequences

| SEQ ID NO | Sequence Description |
|---|---|
| 1 | Fibrillin 1 - Homo Sapiens // Protein |
| 2 | Fibrillin 1 - Homo Sapiens // Nucleotide (genomic) |
| 3 | Fibrillin 1 - Homo Sapiens // Nucleotide (cDNA) |
| 4 | TRPC4 - Homo Sapiens // Protein |
| 5 | TRPC4 - Homo Sapiens // Protein |
| 6 | TRPC4 - Homo Sapiens // Nucleotide (genomic) |
| 7 | TRPC4 - Homo Sapiens // Nucleotide (cDNA) |
| 8 | Fibrillin 1 - Mus Musculus // Protein |
| 9 | Fibrillin 1 - Mus Musculus // Nucleotide (genomic) |
| 10 | Fibrillin 1 - Mus Musculus // Nucleotide (cDNA) |
| 11 | TRPC4 - Mus Musculus // Protein |
| 12 | TRPC4 - Mus Musculus // Nucleotide (genomic) |
| 13 | TRPC4 - Mus Musculus // Nucleotide (cDNA) |

1. (canceled)

2. A method of slowing and/or inhibiting disease progression, the method comprising:
administering to a subject having a hereditary aortopathy a therapeutically effective amount of an angiotensin II receptor antagonist; and
administering to the subject a therapeutically effective amount of a TRPC4 antagonist,
wherein the subject has an aortic aneurysm,
wherein following the administering steps
(i) the aortic aneurysm does not grow and/or
(ii) the subject does not develop an aortic dissection.

3. The method of claim 2, further comprising repeating one or more times the administering of the therapeutically effective amount of the angiotensin II receptor antagonist, the TRPC4 antagonist, or the combination thereof.

4. The method of claim 2, wherein the angiotensin II receptor antagonist is administered prior to the TRPC4 antagonist, concurrently with the TRPC4 antagonist, or after the TRPC4 antagonist.

5. The method of claim 2, wherein the subject has been diagnosed with Marfan syndrome or is suspected of having Marfan syndrome.

6. The method of claim 2, further comprising measuring the size of the aortic aneurysm.

7. (canceled)

8. (canceled)

9. The method of claim 2, wherein the angiotensin II receptor antagonist comprises azilsartan or azilsartan medoxomil, candesartan, eprosartan, irbesartan, losartan, olmesartan, saprisartan, telmisartan, valsartan, any pharmaceutical salt thereof, or any combination thereof.

10. The method of claim 2, wherein the TRPC4 antagonist comprises ML204, HC-070, or the combination thereof.

11. The method of claim 2, wherein the angiotensin II receptor comprises losartan and the TRPC4 antagonist comprises ML204.

12. The method of claim 2, wherein the therapeutically effective dose of the angiotensin II receptor antagonist comprises about 50 mg/kg/day.

13. The method of claim 2, wherein the therapeutically effective dose of a disclosed TRPC4 antagonist comprises about 10 mg/kg/day.

14. The method of claim 6, further comprising surgically repairing the aortic aneurysm.

15. The method of claim 14, wherein surgically repairing the aortic aneurysm comprises replacing the subject's aortic valve, the subject's aortic root, or the combination thereof.

16. The method of claim 2, further comprising measuring one or more biomarkers in a biosample obtained in a subject.

17. The method of claim 16, wherein the one or more biomarkers comprise circTGFB2R, MiR-29a, or both.

18. The method of claim 17, wherein
(i) if the level of circTGFB2R in the subject's biosample is similar to a reference level of circTGFB2R, then the risk of the subject developing an aortic aneurysm and/or experiencing an aortic dissection is minimized, or
(ii) if the level of circTGFB2R in the subject's biosample is less than the reference level of circTGFB2R, then the risk of the subject developing an aortic aneurysm and/or experiencing an aortic dissection is increased.

19. The method of claim 17, wherein
(i) if the level of MiR-29a in the subject's biosample is similar to a reference level of MiR-29a, then the risk of the subject developing an aortic aneurysm and/or experiencing an aortic dissection is minimized, or
(ii) wherein if the level of MiR-29a in the subject's biosample is greater than the reference level of MiR-29a, then the risk of the subject developing an aortic aneurysm and/or experiencing an aortic dissection is increased.

20. The method of claim 1, wherein administering comprises oral administration, intravenous administration, or both.

21. (canceled)

22. The method of claim 2, further comprising reducing the subject's need for surgical intervention.

23. The method of claim 2, wherein the subject's vascular smooth muscle cells (VSMCs) are prevented from switching from a contractile phenotype to a synthetic phenotype.

24. The method of claim 2, wherein the life expectancy of the subject in increased.
